(19) United States
(12) Patent Application Publication
(10) Pub. No.: US 2023/0192861 A1
(43) Pub. Date: Jun. 22, 2023

(54) ANTI-PD-L1/ANTI-B7-H3 MULTISPECIFIC ANTIBODIES AND USES THEREOF

(71) Applicants: ABL BIO INC., Seongnam-si (KR); I-MAB, Shanghai (CN)

(72) Inventors: Eunsil Sung, Seongnam-si (KR); Eunyoung Park, Seongnam-si (KR); Jaehyoung Jeon, Seongnam-si (KR); Junhyun Jeong, Seongnam-si (KR); Daehae Song, Seongnam-si (KR); Sunju Lee, Seongnam-si (KR); Lei Fang, Shanghai (CN); Wenqing Jiang, Shanghai (CN); Feifei Cui, Shanghai (CN)

(21) Appl. No.: 17/778,382

(22) PCT Filed: Nov. 23, 2020
(86) PCT No.: PCT/IB2020/061012

§ 371 Date: May 19, 2022

(30) Foreign Application Priority Data
Nov. 22, 2019 (WO) PCT/CN2019/120246

(51) Int. Cl. C07K 16/28; A61P 35/00

(52) U.S. Cl. CPC C07K 16/2827; C07K 16/2878; A61P 35/00; C07K 2317/31; C07K 2317/24; C07K 2317/21; C07K 2317/92; C07K 2317/33; C07K 2317/622; C07K 2317/55; C07K 2317/732; A61K 2039/505

(57) ABSTRACT

The present disclosure provides an anti-PD-L1/anti-B7-H3 multispecific antibody capable to effectively block the interaction between PD-L1 and its receptor PD-1, and suppress the T-cell inhibitory effect of B7-H3 protein. The multispecific antibody may have high binding affinity to both of a PD-L1 protein and a B7-H3 protein.

### TECHNICAL FIELD

Provided are anti-PD-L1/anti-B7-H3 multispecific antibodies and uses thereof.

### BACKGROUND ART

Programmed death-ligand 1 (PD-L1), also known as cluster of differentiation 274 (CD274) or B7 homolog 1 (B7-H1), is a 40 kDa type 1 transmembrane protein believed to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2.

It has been shown that upregulation of PD-L1 may allow cancers to evade the host immune system. An analysis of tumor specimens from patients with renal cell carcinoma found that high tumor expression of PD-L1 was associated with increased tumor aggressiveness and an increased risk of death. Many PD-L1 inhibitors are in development as immuno-oncology therapies and are showing good results in clinical trials.

B7-H3 (CD276) is a member of the B7 family, and is a transmembrane protein containing an extracellular domain, transmembrane domain and intracellular domain. The two extracellular domains of B7-H3 consist of a single pair (2Ig B7-H3) or two identical pairs (4Ig B7-H3) of immunoglobulin variable domain and immunoglobulin constant domain due to exon duplication. The functional difference between these two forms was not confirmed. The intracellular domain of B7-H3 is short and there is no known motif (Chapoval A I, Ni J, Lau J S, Wilcox R A, Flies D B, Liu D, et al. NatImmunol 2001; 2:269-74.).

The B7-H3 protein has 20˜27% amino acid sequence identity with other members of the B7 family. The human B7-H3 has 88% amino acid sequence identity with the mouse B7-H3. While the mouse B7-H3 has one subtype (2IgB7-H3), the human B7-H3 has two subtypes (2Ig B7-H3, 4Ig B7-H3). 4Ig B7-H3 was confirmed in a human tissue.

It was found that the mouse B7-H3 bound to TLT1 of a CD8+ T cell, thereby enhancing T cell proliferation, cytokine production and cytotoxicity, and thus it was suggested that TLT2 could act as a B7-H3 receptor. Subsequently, however, evidence for this interaction has not been found in either mouse or human (M. Loos, D. M. Hedderich, and D. M. Hedderich, et al. BMC Cancer, vol. 9, article 463, 2009).

The B7-H3 protein is not always expressed in natural killer cells (NK cells) or antigen presenting cells (APC) in a normal tissue, but its expression can be induced. Although the expression of B7-1 and B7-2 is mostly limited to immunocytes such as the antigen presenting cells, the B7-H3 protein is present in not only osteoblasts, fibroblasts, fibroblast-like synovial cells and epithelial cells but also liver, lung, bladder, testis, prostate, breast, placenta and lymphatic vessel organs of human. This wide expression pattern suggests more various immunological and non-immunologic functions for B7-H3, particularly in peripheral tissues.

In recent years, B7-H3 expression has been confirmed in various solid cancers such as non-small cell lung cancer, renal cell carcinoma, neuroblastoma, colorectal cancer, pancreatic cancer, gastric cancer, lung cancer, prostate cancer, endometrial cancer, hepatocellular carcinoma, breast cancer, cervical cancer, osteosarcoma, oral cancer, bladder cancer, glioma, melanoma, etc., and it has been reported that it is expressed in hematologic malignancies such as acute leukemia, multiple myeloma, various kinds of lymphomas (Zhimeng Yea, Zhuojun Zhengb et al, Cell Physiol Biochem (2016), Elodie Picarda, Kim C. Ohaegbulam and Xingxing Zang, clinical cancer research (2016), Wei Zhang, Yanfang Wang, Jing Wang et al, international journal of oncology (2015)).

B7-H3 is a protein which belongs to an immune checkpoint ligand. The immune checkpoint protein acts to control immunocytes in a human body to prevent them from making false abnormal behavior. When the immune checkpoint protein is overexpressed in a cancer cell, immunocytes receive an abnormal signal which the cancer cell sends as a normal signal, and recognize the cancer cell as a healthy cell. An immune checkpoint inhibitor is an anti-cancer immune-therapeutic agent which blocks such an abnormal signal of the cancer cell, thereby treating cancer by immunologic function of a patient himself B7-H3 which is one of the immune checkpoint ligands binds to a B7-H3 receptor on a T cell surface and induces inhibition of immunoreaction of the T cell, but it is still not revealed what receptor B7-H3 binds.

An antibody which can block such an immune checkpoint ligand shows anti-cancer immune-therapeutic effect by partially or completely neutralizing interaction of immune checkpoint ligands and immune checkpoint receptors and inhibiting immune checkpoint, thereby reactivating the degraded activity of immunocytes. A receptor binding to B7-H3 is not been found yet, but an anti-B7-H3 antibody binding to B7-H3 can show an anti-cancer immune-therapeutic effect by blocking binding between the immune checkpoint receptor and B7-H3 and inhibiting such an immune checkpoint, thereby reactivating the degraded activity of immunocytes. In other words, the anti-B7-H3 monoclonal antibody blocking the binding to the B7-H3 receptor may be expected to have an anti-cancer therapeutic effect. (Elodie Picarda, Kim C. Ohaegbulam and Xingxing Zang, Clin Cancer Res, 2017 Jul. 12; 22). U.S. Pat. Nos. 8,802,091 and 9,371,395 disclose antibodies to B7-H3.

### DISCLOSURE

### Technical Problem

Provided are anti-PD-L1/anti-B7-H3 multispecific antibodies and uses thereof.

### Technical Solution

The present disclosure provides anti-PD-L1/anti-B7-H3 multispecific antibodies capable to effectively block the interaction between PD-L1 and its receptor PD-1, and suppress the T-cell inhibitory effect of B7-H3 protein. The disclosed multispecific antibodies may have high binding affinity to both PD-L1 (e.g., a human PD-L1 protein) and B7-H3 (e.g., a human B7-H3 protein). The present disclosure also provides combination therapies that includes an anti-PD-L1 antibody and separately an anti-B7-H3 antibody.

As the experimental data demonstrate, the combination treatment of an anti-B7-H3 antibody and an anti-PD-1 antibody exhibited superior cancer growth inhibition efficacy a syngeneic manner, as compared to single administrations. Further, the combinatory effect was even more pronounced with anti-B7-H3/anti-PD-L1 bispecific antibodies.

Moreover, among all of the bispecific antibodies tested, the “1+1” formats considerably outformed the “2+2” formats. Such a result was also surprising because the “2+2” formats were thought to be more potent since each molecule has more binding sites to both PD-L1 and B7-H3 and may be structurally more stable.

The superior activities of the anti-B7-H3/anti-PD-L1 bispecific antibodies, in particular those of the “1+1” formats, are contemplated to be attributed to how the B7-H3 and the PD-L1 proteins are expressed on target cancer cells. It is also contemplated that the epitopes of the anti-B7-H3 antibodies and/or the anti-PD-L1 antibodies contributed to the significant synergism. For instance, unlike all other known therapeutic anti-PD-L1 antibodies, the anti-PD-L1 antibodies and fragments of the instant disclosure bind to the IgC domain of the PD-L1 protein. In one embodiment, therefore, provided is an anti-PD-L1/anti-B7-H3 bispecific antibody, comprising an anti-PD-L1 unit having binding specificity to a human PD-L1 protein and an anti-B7-H3 unit having binding specificity to a human B7-H3 protein. The bispecific antibody preferably has a 1+1 format, but can also take a 2+2 format as further described below.

In a 1+1 format, for instance, the bispecific antibody has a Fc fragment and both the anti-PD-L1 and anti-B7-H3 binding fragments are at the N-terminal side of the Fc fragment (or alternatively at the C-terminal side of the Fc fragment). Each of the anti-PD-L1 and anti-B7-H3 binding fragments can be independently selected from a Fab fragment, a single chain Fab fragment (scFab), a single-domain antibody (sdAb), a single chain variable fragment (scFv), and antigen-binding moiety, or any other antigen-binding fragments.

In one example, the PD-L1 binding site is a Fab fragment and the B7-H3 binding site is a scFab fragment. In one example, the PD-L1 binding site is a Fab fragment and the B7-H3 binding site is a scFv fragment. In one example, the PD-L1 binding site is a scFab fragment and the B7-H3 binding site is a Fab. In one example, the PD-L1 binding site is a scFv fragment and the B7-H3 binding site is a Fab. A 1+1 format, as the name suggests, is monovalent for PD-L1 binding and monovalent for B7-H3 binding.

In a 2+2 format, a full antibody (Fab and Fc) can be fused to two antigen-binding fragments at the C-terminal side of the Fc fragment. In one embodiment, the full antibody is specific to PD-L1 and the two antigen-binding fragments are specific to B7-H3. In one embodiment, the full antibody is specific to B7-H3 and the two antigen-binding fragments are specific to PD-L1. The antigen-binding fragments can be selected from a Fab fragment, a single chain Fab fragment (scFab), a single-domain antibody (sdAb), a single chain variable fragment (scFv), and antigen-binding moiety, or any other antigen-binding fragments.

In any of the above examples, the anti-PD-L1 binding unit can specifically bind to an immunoglobulin C (Ig C) domain of the human PD-L1 protein, wherein the Ig C domain consists of amino acid residues 133-225. In some embodiments, the anti-PD-L1 binding unit can specifically bind to amino acid residues Y134, K162, and N183 of the human PD-L1 protein. The anti-PD-L1/anti-B7-H3 multispecific antibody may comprise an anti-PD-L1 antibody or an antigen-binding fragment thereof as a PD-L1 targeting moiety, which is capable of specifically recognizing and/or binding to a PD-L1 protein; and an anti-B7-H3 antibody or an antigen-binding fragment thereof as a B7-H3 targeting moiety, which is capable of specifically recognizing and/or binding to a B7-H3 protein.

The anti-PD-L1/anti-B7-H3 multispecific antibody may comprise an anti-PD-L1 antibody or an antigen-binding fragment thereof as a PD-L1 targeting moiety.

In an embodiment, the anti-PD-L1 antibody or fragment thereof comprised in the multispecific antibody can specifically bind to an immunoglobulin C (IgC) domain of PD-L1 (e.g., human PD-L1) protein. In some embodiments, the IgC domain consists of amino acid residues 133-225 of a human PD-L1 protein. In some embodiments, the anti-PD-L1 antibody or fragment thereof can bind to at least one of amino acid residues selected from Y134, K162, and N183 of a human PD-L1 protein. In some embodiments, the anti-PD-L1 antibody or fragment thereof does not bind to an immunoglobulin V (IgV) domain of the PD-L1 protein, and for example, the IgV domain consists of amino acid residues 19-127 of a human PD-L1 protein

The anti-PD-L1/anti-B7-H3 multispecific antibody, comprising an anti-PD-L1 antibody or an antigen-binding fragment thereof and an anti-B7-H3 antibody or an antigen-binding fragment thereof,

wherein the anti-PD-L1 antibody or fragment thereof comprises (1) a VH CDR1 having an amino acid sequence selected from the group consisting of SEQ ID NOs: 1 and 294; (2) a VH CDR2 having an amino acid sequence selected from the group consisting of SEQ ID NOs: 2, 3 and 295; (3) a VH CDR3 having an amino acid sequence selected from the group consisting of SEQ ID NOs: 4, 5, 6, 7, 8, 9, 10, 11 and 296; (4) a VL CDR1 having an amino acid sequence selected from the group consisting of SEQ ID NOs: 12, 13, 14 and 297; (5) a VL CDR2 having an amino acid sequence selected from the group consisting of SEQ ID NOs: 15 and 298; and (6) a VL CDR3 having an amino acid sequence selected from the group consisting of SEQ ID NOs: 16, 17, 18, 19 and 299; and

the anti-B7-H3 antibody or fragment thereof comprises (1) a VH CDR1 having an amino acid sequence selected from the group consisting of SEQ ID NOs: 20, 21, 22 and 23; (2) a VH CDR2 having an amino acid sequence selected from the group consisting of SEQ ID NOs: 24, 25, 26, 27, 28 and 29; and (3) VH CDR3 having an amino acid sequence selected from the group consisting of SEQ ID NOs: 30, 31, 32, 33 and 34; (4) a VL CDR1 having an amino acid sequence selected from the group consisting of SEQ ID NOs: 35, 36, 37, 38 and 39; (5) a VL CDR2 having an amino acid sequence selected from the group consisting of SEQ ID NOs: 40, 41, 42, 43, 44 and 45; and (6) a VL CDR3 having an amino acid sequence selected from the group consisting of SEQ ID NOs:46, 47, 48, 49, and 50.

In an embodiment, the anti-PD-L1 antibody or fragment thereof comprises (1) a VH CDR1 having an amino acid sequence selected from the group consisting of SEQ ID NOs: 1 and 294; (2) a VH CDR2 having an amino acid sequence selected from the group consisting of SEQ ID NOs: 2, 3 and 295; (3) a VH CDR3 having an amino acid sequence selected from the group consisting of SEQ ID NOs: 4, 5 and 296; (4) a VL CDR1 having an amino acid sequence selected from the group consisting of SEQ ID NOs: 12 and 297; (5) a VL CDR2 having an amino acid sequence selected from the group consisting of SEQ ID NOs: 15 and 298; and (6) a VL CDR3 having an amino acid sequence selected from the group consisting of SEQ ID NOs: 16 and 299; and

the anti-B7-H3 antibody or fragment thereof comprises (1) a VH CDR1 having an amino acid sequence selected from the group consisting of SEQ ID NOs: 20 and 21; (2) a VH CDR2 having an amino acid sequence selected from the group consisting of SEQ ID NOs: 24 and 25; and (3) VH CDR3 having an amino acid sequence selected from the group consisting of SEQ ID NOs: 30 and 31; (4) a VL CDR1 having an amino acid sequence selected from the group consisting of SEQ ID NOs: 35 and 36; (5) a VL CDR2 having an amino acid sequence selected from the group consisting of SEQ ID NOs: 40 and 41; and (6) a VL CDR3 having an amino acid sequence selected from the group consisting of SEQ ID NOs:46 and 47.

In an embodiment, the anti-PD-L1 antibody or fragment thereof comprises (1) a VH CDR1 having an amino acid sequence selected from the group consisting of SEQ ID NOs: 1 and 294; (2) a VH CDR2 having an amino acid sequence selected from the group consisting of SEQ ID NOs: 3 and 295; (3) a VH CDR3 having an amino acid sequence selected from the group consisting of SEQ ID NOs: 5 and 296; (4) a VL CDR1 having an amino acid sequence selected from the group consisting of SEQ ID NOs: 12 and 297; (5) a VL CDR2 having an amino acid sequence selected from the group consisting of SEQ ID NOs: 15 and 298; and (6) a VL CDR3 having an amino acid sequence selected from the group consisting of SEQ ID NOs: 16 and 299; and

the anti-B7-H3 antibody or fragment thereof comprises (1) a VH CDR1 having an amino acid sequence selected from the group consisting of SEQ ID NOs: 20 and 21; (2) a VH CDR2 having an amino acid sequence selected from the group consisting of SEQ ID NOs: 24 and 25; and (3) VH CDR3 having an amino acid sequence selected from the group consisting of SEQ ID NOs: 30 and 31; (4) a VL CDR1 having an amino acid sequence selected from the group consisting of SEQ ID NOs: 35 and 36; (5) a VL CDR2 having an amino acid sequence selected from the group consisting of SEQ ID NOs: 40 and 41; and (6) a VL CDR3 having an amino acid sequence selected from the group consisting of SEQ ID NOs:46 and 47.

In an embodiment, the anti-PD-L1 antibody or fragment thereof comprises a light chain variable region having an amino acid sequence selected from the group consisting of SEQ ID NOs: 122, 124, 126, 128, 130, 132, 134, 136, 138, 140 and 209; or a peptide having at least 90% sequence identity to an amino acid sequence selected from the group consisting of SEQ ID NOs: 122, 124, 126, 128, 130, 132, 134, 136, 138, 140 and 209.

In an embodiment, the anti-PD-L1 antibody or fragment thereof comprises a light chain variable region having an amino acid sequence selected from the group consisting of SEQ ID NOs: 130 and 209; or a peptide having at least 90% sequence identity to an amino acid sequence selected from the group consisting of SEQ ID NOs: 130 and 209.

In an embodiment, the anti-B7-H3 antibody or fragment thereof comprises a light chain variable region having an amino acid sequence selected from the group consisting of SEQ ID NOs: 57, 58, 59, 60, 61 and 62; or a peptide having at least 90% sequence identity to an amino acid sequence selected from the group consisting of SEQ ID NOs: 57, 58, 59, 60, 61 and 62.

In an embodiment, the anti-B7-H3 antibody or fragment thereof comprises a light chain variable region having an amino acid sequence selected from the group consisting of SEQ ID NOs: 57 and 58; or a peptide having at least 90% sequence identity to an amino acid sequence selected from the group consisting of SEQ ID NOs: 57 and 58.

In an embodiment, the anti-PD-L1 antibody or fragment thereof comprises a heavy chain variable region having an amino acid sequence selected from the group consisting of SEQ ID NOs: 121, 123,125, 127, 129, 131, 133, 135, 137, 139 and 211; or a peptide having at least 90% sequence identity to an amino acid sequence selected from the group consisting of SEQ ID NOs: 121, 123,125, 127, 129, 131, 133, 135, 137, 139 and 211.

In an embodiment, the anti-PD-L1 antibody or fragment thereof comprises a heavy chain variable region having an amino acid sequence selected from the group consisting of SEQ ID NOs: 129 and 211; or a peptide having at least 90% sequence identity to an amino acid sequence selected from the group consisting of SEQ ID NOs: 129 and 211.

In an embodiment, the anti-B7-H3 antibody or fragment thereof comprises a heavy chain variable region having an amino acid sequence selected from the group consisting of SEQ ID NOs: 51, 52, 53, 54, 55 and 56; or a peptide having at least 90% sequence identity to an amino acid sequence selected from the group consisting of SEQ ID NOs: 51, 52, 53, 54, 55 and 56.

In an embodiment, the anti-B7-H3 antibody or fragment thereof comprises a heavy chain variable region having an amino acid sequence selected from the group consisting of SEQ ID NOs: 51 and 52; or a peptide having at least 90% sequence identity to an amino acid sequence selected from the group consisting of SEQ ID NOs: 51 and 52.

In an embodiment, the anti-PD-L1 antibody or antigen-binding fragment thereof is capable of binding to at least one of amino acid residues selected from Y134, K162, and N183 of the PD-L1 protein.

In an embodiment, the anti-PD-L1 antibody or antigen-binding fragment thereof does not bind to an immunoglobulin V (Ig V) domain of the PD-L1 protein, wherein the Ig V domain consists of amino acid residues 19-127.

In an embodiment, the anti-B7-H3 antibody or antigen-binding fragment thereof reactivates an activity of a T cell inhibited by a B7-H3 immune checkpoint.

In an embodiment, each of the anti-PD-L1 antibody or antigen-binding fragment thereof and the anti-B7-H3 antibody or antigen-binding fragment thereof is independently a chimeric antibody, a humanized antibody, or a fully human antibody.

In an embodiment, each of the anti-PD-L1 antibody or antigen-binding fragment thereof and the anti-B7-H3 antibody or antigen-binding fragment thereof is independently selected from a group consisting of a whole IgG, Fab, Fab′, F(ab′)2, scFab, dsFv, Fv, scFv, scFv-Fc, scFab-Fc, diabody, minibody, scAb, dAb, half-IgG and combinations thereof.

The anti-PD-L1/anti-B7-H3 multispecific antibody of the present invention is in the form of IgG X scFv form.

In other embodiment, the anti-PD-L1/anti-B7-H3 multispecific antibody of the present invention is in the form of (HC+LC) X scFab-Fc form.

In an embodiment, the anti-PD-L1/anti-B7-H3 multispecific antibody of the present invention further comprises an anti-4-1BB antibody or an antigen-binding fragment thereof. The anti-4-1BB antibody or an antigen-binding fragment thereof may be selected from a group consisting of a whole IgG, Fab, Fab′, F(ab′)2, scFab, dsFv, Fv, scFv, scFv-Fc, scFab-Fc, diabody, minibody, scAb, dAb, half-IgG and combinations thereof.

In an embodiment, the polynucleotide may be a polynucleotide encoding a heavy chain and/or light chain variable region disclosed herein.

In an embodiment, an isolated polynucleotide may be a polynucleotide encoding a heavy chain and/or light chain disclosed herein.

In an embodiment, a vector comprising the polynucleotide is provided.

In an embodiment, a cell line transformed by the vector is provided.

Another embodiment provides a method of preparation of an isolated antibody specifically binding to PD-L1 or B7-H3, or its antigen-binding fragment, comprising a step of isolating an antibody or its antigen-binding fragment from the cell line.

Another embodiment provides a pharmaceutical composition comprising the anti-PD-L1/anti-B7-H3 multispecific antibody of the present invention and a pharmaceutically acceptable carrier.

In an embodiment, the pharmaceutical composition is a pharmaceutical composition for treating and/or preventing a disease associated with PD-L1, B7-H3, or both thereof, for example, cancer.

Another embodiment provides a method for treating cancer in a patient in need thereof, comprising administering to the patient an effective amount of the anti-PD-L1/anti-B7-H3 multispecific antibody of the present invention.

Another embodiment provides a method of detection of PD-L1 or B7-H3 in a biological sample, comprising a step of contacting an antibody or antigen-binding fragment thereof as described herein with a biological sample requiring detection of PD-L1 or B7-H3 expression. The method may further comprise, after the step of contacting, a step of measuring an antigen-antibody response in the biological sample treated (contacted) with the antibody or antigen-binding fragment thereof.

In an embodiment, the method may be performed in vitro or in vivo.

In other embodiment, a kit comprising the antibody or its antigen-binding fragment or the composition comprising the antibody or antigen-binding fragment is provided. The kit may be provided as a kit for PD-L1 or B7-H3 detection or a kit for administration for cancer treatment or a kit for cancer treatment, depending on a specific purpose for which the kit is used, and depending on its specific purpose, an additional component may be comprised. For example, a component for immunological analysis, for example, buffer and instructions for a kit for detection or diagnosis, or an apparatus for administration and instructions for a kit for antibody administration or cancer treatment may be further comprised.

The anti-B7-H3 antibody or antigen-binding fragment thereof may (1) specifically recognize or bind to a B7-H3 expressed on a cell surface derived from human, mouse, or monkey or (2) specifically recognize or bind to an extracellular domain of B7-H3 which may or may not be present on a cell surface.

The anti-PD-L1/anti-B7-H3 multispecific antibody of the present invention shows an effect as an immune checkpoint inhibitor which activates a T cell of which activity is degraded by an immune checkpoint ligand, B7-H3 protein, thereby being usefully used for cancer treatment through activation of immunocytes.

Furthermore, the antibody may be used for example, for drug delivery to specific cancer, etc., or detection, diagnosis and/or targeting of cancer by specific binding.

In addition, the monoclonal antibody disclosed herein has cross-species reactivity having the binding affinity to human, monkey and mouse B7-H3. This may be very useful for development of drugs, etc., compared to other human antibodies which do not show the binding affinity to mouse or monkey B7-H3. For example, the monoclonal antibody or various forms of therapeutic agents using the antibody can progress the development of drugs more economically and effectively by obtaining the initial result in a low cost of mouse model, before progressing a high cost of monkey-based experiment.

Another embodiment provides a use of anti-PD-L1/anti-B7-H3 multispecific antibody of the present invention in the manufacture of medicament for treating or preventing a disease associated with PD-L1, B7-H3, or both thereof.

Another embodiment provides a use of anti-PD-L1/anti-B7-H3 multispecific antibody of the present invention for treating or preventing a disease associated with PD-L1, B7-H3, or both thereof.

### Advantageous Effects

The antibody or the antigen binding fragment thereof of the present invention that specifically binds to PD-L1 and B7-H3 shows an excellent cancer cell proliferation inhibitory activity and a remarkably excellent anticancer activity in a synergistic manner, thus effectively preventing or treating the disease such as cancer.

### DESCRIPTION OF DRAWINGS

FIG. 1a shows the “2+2 format” of the anti-PD-L1/anti-B7-H3 multispecific antibody of the present invention.

FIG. 1b shows the “1+1 format” of the anti-PD-L1/anti-B7-H3 multispecific antibody of the present invention.

FIG. 2 schematically shows the mechanism of action of an anti-PD-L1/anti-B7-H3 multispecific antibody according to an embodiment.

FIG. 3 plots demonstrated selection criteria for PD-L1 variants in order to identify required residues for Hu1210-41 binding.

FIG. 4 shows the locations of Y134, K162, and N183, the residues (spheres) involved in binding to the anti-PD-L1 antibody according to an embodiment.

FIGS. 5a and 5b are the results of analysis (ELISA) of the binding capacity of the anti-B7-H3 antibody prepared according to one embodiment of the present invention to the extracellular domain (ECD) of B7-H3 protein. It was shown that every antibody bound to the extracellular domain of human B7-H3 protein in a concentration-dependent manner.

FIG. 6 is the result of analysis (ELISA) of the binding capacity of the anti-B7-H3 antibody prepared according to one embodiment of the present invention to ECD of the other proteins belonging to B7 family. It was shown that every antibody prepared according to one embodiment of the present invention did not bind to the other proteins and specifically recognized B7-H3 protein only.

FIG. 7 is the result of analyzing cross-species reactivity of the anti-B7-H3 antibody prepared according to one embodiment of the present invention by ELISA. It was shown that every antibody bound to monkey (cynomolgus) B7-H3 and mouse B7-H3 in a concentration-dependent manner.

FIG. 8 is the result of comparing the binding capacity degree of various anti-B7-H3 antibodies prepared according to one embodiment of the present invention to mouse B7-H3 protein by ELISA. It was shown that the binding degree of antibodies to mouse B7-H3 are varied, but every antibody bound to mouse B7-H3 protein in a concentration-dependent manner. By Contrast, 84D antibody used as a comparison group antibody did not bind to mouse B7-H3 protein.

FIG. 9 is the result of measurement (FACS) for the binding capacity of the anti-B7-H3 antibody prepared according to one embodiment of the present invention to cell surface expression B7-H3 antigen. MCF-7 cell line is a cell line overexpressing B7-H3, and Jurkat is a cell line which does not express B7-H3. It was shown that the anti-B7-H3 antibodies of the present invention specifically bound to MCF-7, the cell line overexpressing B7-H3, but did not bind to Jurkat, the cell line which does not express B7-H3.

FIG. 10 is the result of measurement (FACS) for the binding capacity of the anti-B7-H3 antibody prepared according to one embodiment of the present invention to B7-H3 antigen expressed on the cell surface, for varying antibody concentrations. It was shown that every antibody bound to B7-H3 expressing cancer cell lines (MCF-7, DLD-1, HCC1954, and HCT116) in a concentration-dependent manner. The binding capacity of the antibodies to B7-H3 expressed in the other various cancer cell lines is described in Table 19.

In order that an antibody to a specific antigen is used in vivo as an antibody for treatment, etc., it is a necessary factor to bind to a cell surface expression antigen. In case of some antibodies, they bind to a purified antigen, but do not bind to an antigen expressed on the cell surface. In this case, the antibody administered into a body cannot bind to a cell in the body and therefore it is not possible to act in vivo as an antibody for treatment, etc. Thus, this result shows that the anti-B7-H3 antibody of the present invention can bind to cell surface B7-H3 and show activity in vivo, thereby being usefully used as an antibody for treatment.

FIG. 11 is the result of measurement (FACS) for the binding capacity of the anti-B7-H3 antibody to mouse-derived cancer cell lines (CT26, B16F10, and TC-1). It was shown that every B7-H3 monoclonal antibody specifically recognized B7-H3 expressed on a surface of mouse-derived cancer cell lines, too.

FIG. 12 is the result of measurement for the ADCC-inducing capacity of the anti-B7-H3 antibody prepared according to one embodiment of the present invention. The antibody prepared according to one embodiment of the present invention showed ADCC induction specific to human B7-H3 positive cell lines only, including MCF-7, Calu-6, DLD-1 and Mino. ADCC was not observed in the human B7-H3 negative cell line, Jurkat. This shows that the antibody can be effectively used for death of cancer cells, since it specifically binds only to B7-H3 expressing cancer cells and induces antibody-dependent cell-mediated cytotoxicity. In particular, it shows that the anti-B7-H3 antibody of the present invention can be more effectively used for cancer treatment, since it has a lower EC50 and stronger strength of signal of antibody-dependent cell-mediated cytotoxicity, compared to the comparison antibody, 84D.

FIG. 13a shows the T cell activity inhibited by B7-H3 protein and the consequently inhibited production of interferon gamma. It was shown that the B7-H3 protein inhibited the production of interferon gamma in a concentration-dependent manner.

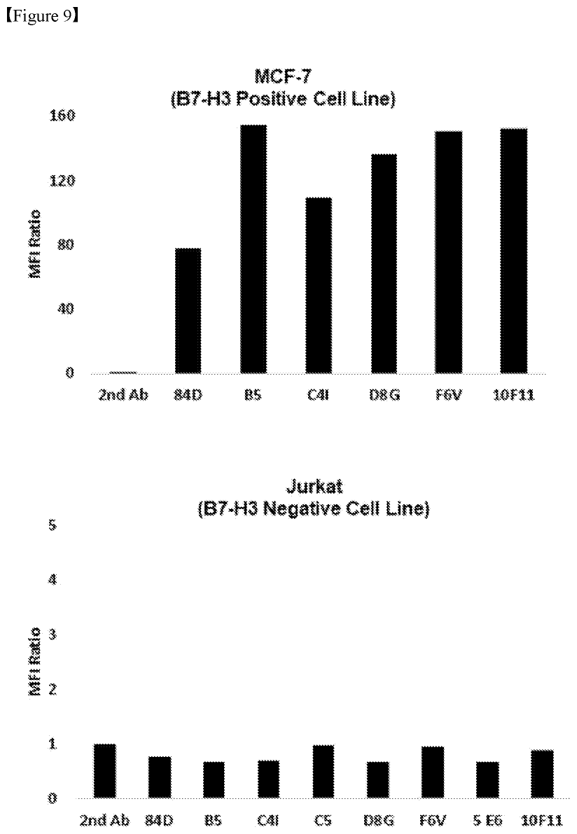

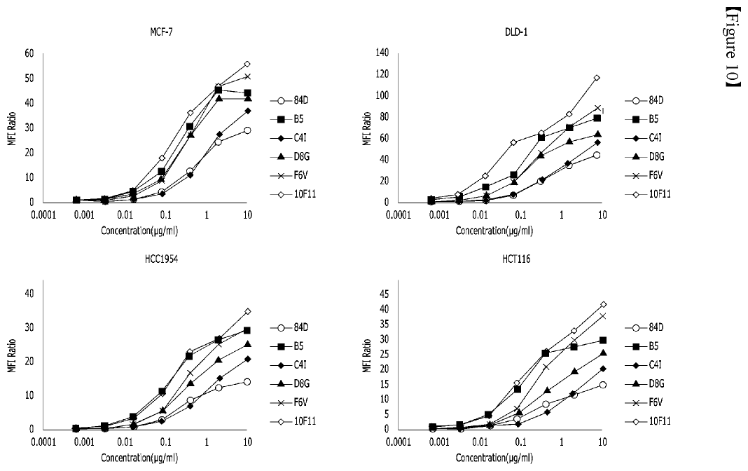

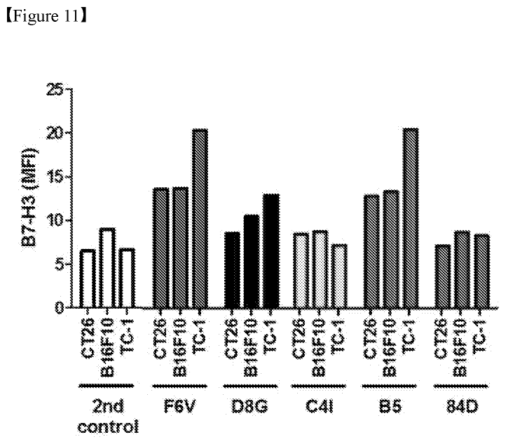

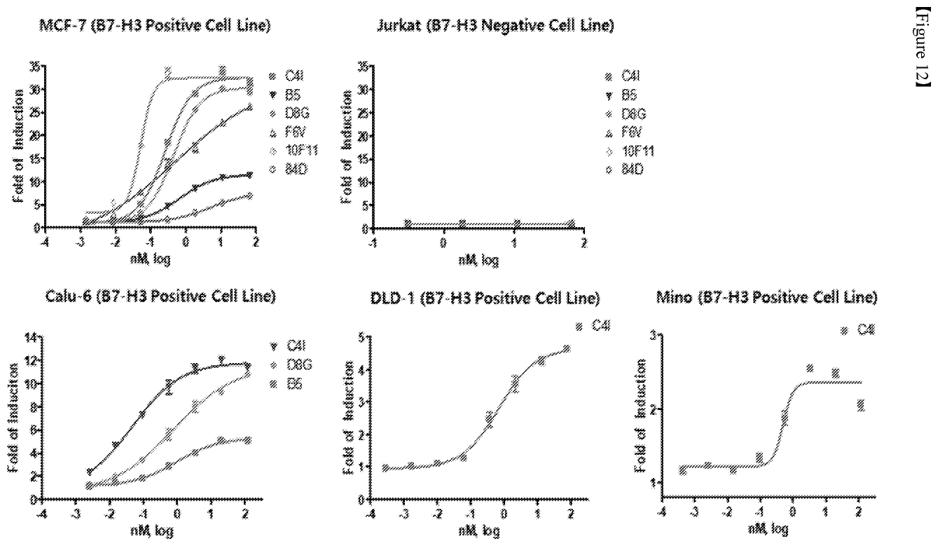

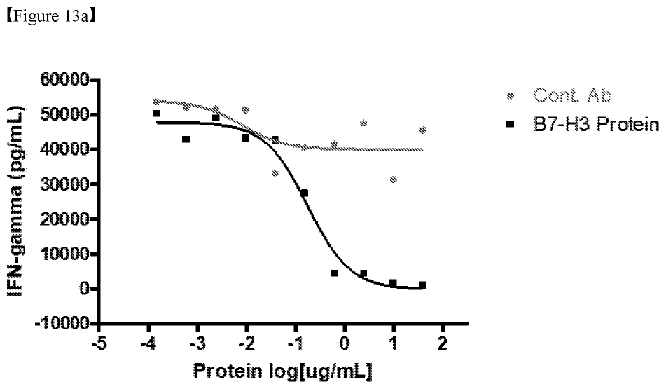

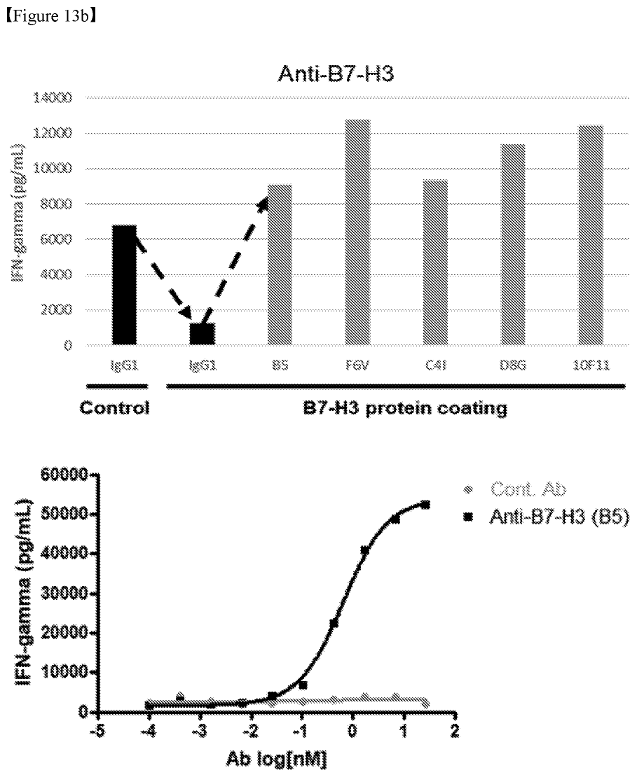

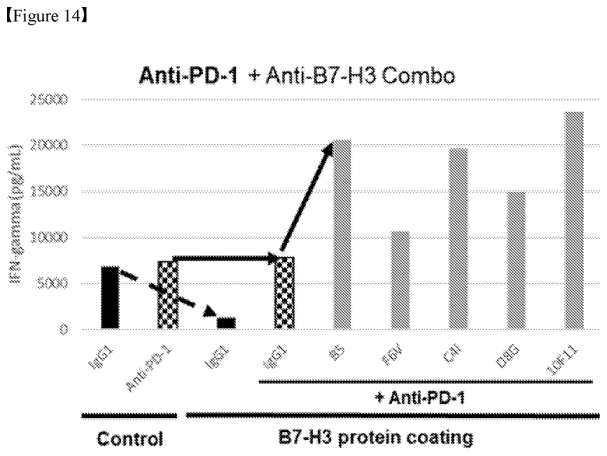

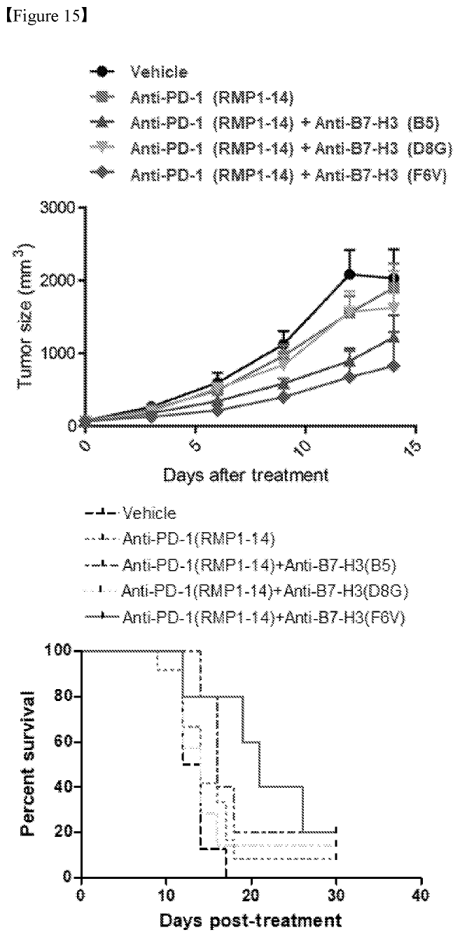

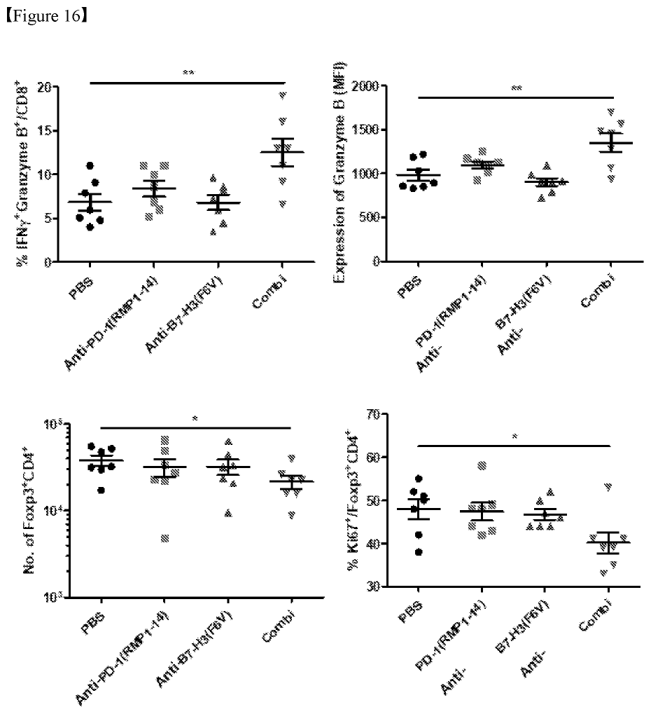

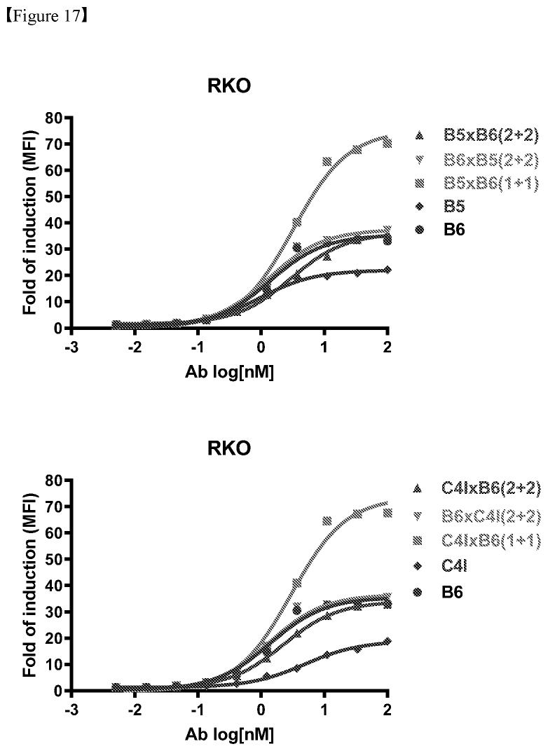

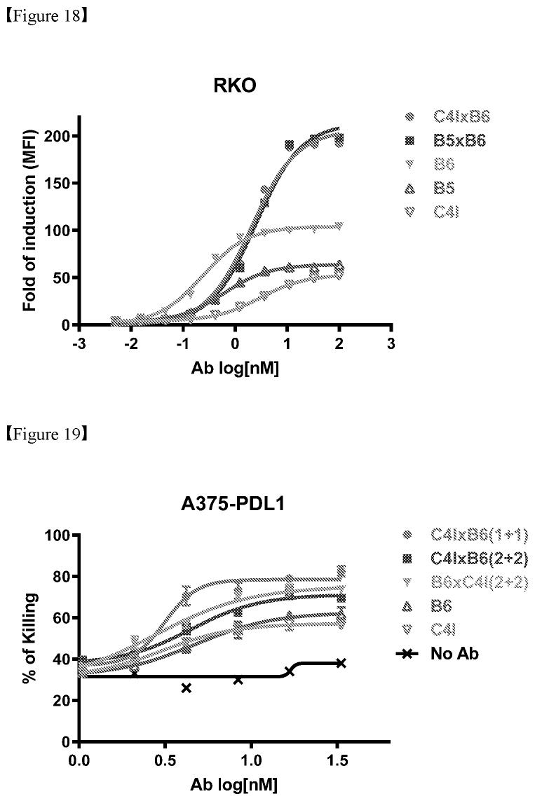

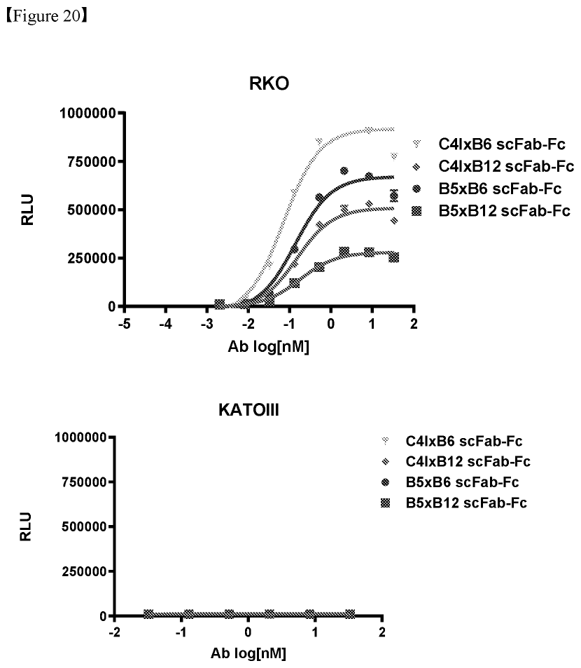

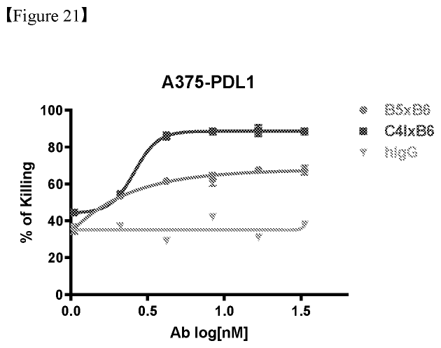

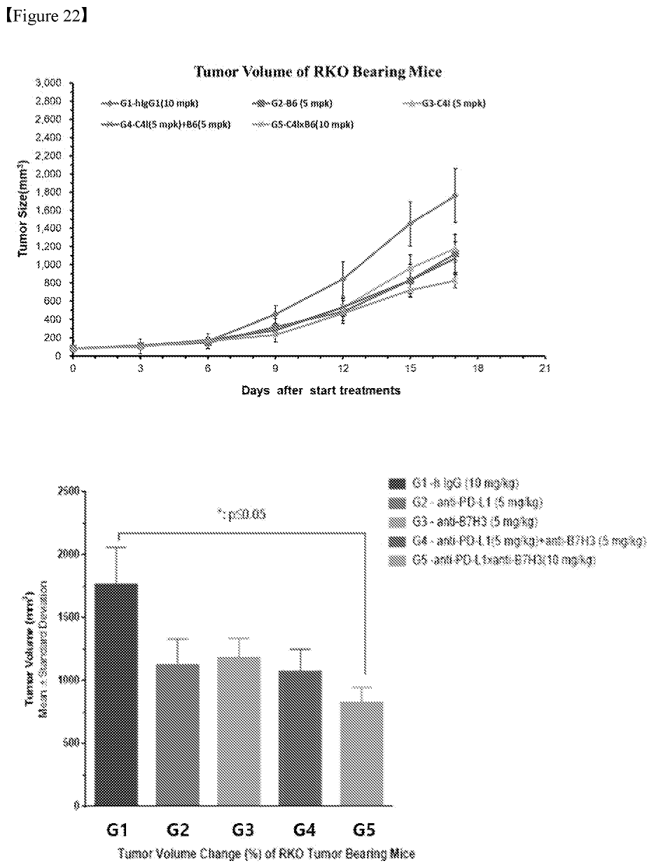

FIG. 13b shows that the anti-B7-H3 antibody prepared according to one embodiment of the present invention can reactivate a T cell activity as inhibited by B7-H3 protein as in FIG. 9a, which was measured by interferon gamma production. The results of FIG. 9a and FIG. 9b mean that the anti-B7-H3 monoclonal antibody of the present invention can neutralize or block immune-suppression of a T cell by B7-H3 protein. In other words, the B7-H3 antibody of the present invention can induce death of a cancer cell by a T cell by reactivating a T cell of which activity is inhibited, and this shows that the B7-H3 antibody of the present invention can be effectively used for cancer treatment.

FIG. 14 shows an influence of the anti-B7-H3 antibody prepared according to one embodiment of the present invention on T cell activation by interferon, when used along with an anti-PD-1 antibody, which was measured by gamma production. It was shown that the anti-B7-H3 antibody, alone or with the anti-cancer immune antibody, effectively facilitates the production of interferon gamma by activating the T cell. This shows that the antibody may be effectively used for treatment of cancer by activating the T cell, alone or when combined with other anti-cancer immune antibody.

FIG. 15 is the result confirming that the tumor growth is inhibited and the survival rate is improved, when the anti-B7-H3 antibody prepared according to one embodiment of the present invention is co-administered with anti-PD-1 antibody in an isogenic tumor transplantation model in which CT26, a mouse B7-H3 positive cancer cell line, is transplanted. Anti-PD-L1 antibody acts through immune checkpoint inhibition. It was shown that the anti-B7-H3 antibody, alone or with the anti-cancer immune antibody, effectively facilitates the production of interferon gamma by activating the T cell. This shows that the antibody may be effectively used for treatment of cancer by activating the T cell, alone or when combined with other anti-cancer immune antibody.

FIG. 16 is the result of analyzing the tumor-infiltrating lymphocytes flow in tumor, when the anti-B7-H3 antibody prepared according to one embodiment of the present invention is co-administered with anti-PD-a antibody in an isogenic tumor transplantation model in which CT26, a mouse B7-H3 positive cancer cell line, is transplanted. It was shown that the activity of CD8+ T cell is increased and the proliferation of a regulatory T cell is inhibited, by co-administration of the anti-B7-H3 antibody and anti-PD-1 antibody. This means that the anti-cancer effect by co-administration of the anti-B7-H3 antibody and the immune checkpoint inhibitor, anti-PD-1 antibody, appears through changes of the CD8+ T cell and regulatory T cell.

FIG. 17 is the result of analysis (FACS) of the binding ability of the anti-PD-L1/anti-B7-H3 bispecific antibodies prepared according to embodiments of the present invention to PD-L1 and B7-H3 which expressed on cell surface.

FIG. 18 is the result of analysis (FACS) of the binding affinity of the 1+1 format anti-PD-L1/anti-B7-H3 bispecific antibodies prepared according to embodiments of the present invention to PD-L1 and B7-H3 which expressed on cell surface.

FIG. 19 is the result of analysis (IG4 TCR-engineered T cell assay) of the in vitro tumor killing potency of the anti-PD-L1/anti-B7-H3 bispecific antibodies prepared according to embodiments of the present invention.

FIG. 20 is the result of analysis of the antibody-dependent cell-mediated cytotoxicity (ADCC) abilities of the 1+1 format anti-PD-L1/anti-B7-H3 bispecific antibodies prepared according to embodiments of the present invention.

FIG. 21 is the result of analysis (IG4 TCR-engineered T cell assay) of the in vitro tumor killing potency of 1+1 format C4I×B6 and B5×B6 bispecific antibodies prepared according to embodiments of the present invention.

FIG. 22 is the result of analysis of tumor growth inhibition of the 1+1 format bispecific antibodies according to embodiments of the present invention using RKO-PBMC humanized mice model.

FIG. 23 is the result of analysis of the ability of trispecific antibodies to promote 4-1BB signal.

### MODE FOR INVENTION

### Definitions

It is to be noted that the term “a” or “an” entity refers to one or more of that entity for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. Moreover, “polypeptide fragment” means a polypeptide having deletion of an amino acid sequence of an amino terminal, deletion of an amino acid sequence of a carboxyl terminal and/or an internal deletion, compared to a full-length protein. This fragment may also include modified amino acids compared to a full-length protein. In one embodiment, the fragment may be about 5 to 900 amino acids in length, for example, at least 5, 6, 8, 10, 14, 20, 50, 70, 100, 110, 150, 200, 250, 300, 350, 400, 450, 500, 550, 600, 650, 700, 750, 800, 850 or more amino acids in length. Considering the purpose of the present invention, the useful polypeptide fragment includes an immunological functional fragment of an antibody comprising an antigen-binding domain. In case of PD-L1 or B7-H3 binding antibody, such a useful fragment includes a CDR sequence comprising 1, 2, or 3 of heavy chains or light chains, or all or a portion of antibody chain comprising a variable region or constant region of a heavy chain or light chain, but not limited thereto.

As used herein, “variant” of a polypeptide such as for example, an antigen-binding fragment, a protein or an antibody is a polypeptide in which one or more amino acid residues are inserted, deleted, added and/or substituted, as compared to another polypeptide sequence, and includes a fusion polypeptide. In addition, a protein variant includes one modified by protein enzyme cutting, phosphorylation or other posttranslational modification, but maintaining biological activity of the antibody disclosed herein, for example, specific binding to B7-H3 and biological activity. The variant may be about 99%, 98%, 97%, 96%, 95%, 94%, 93%, 92%, 91%, 90%, 89%, 88%, 87%, 86%, 85%, 84%, 83%, 82%, 81%, or 80% identical to the sequence of the antibody or its antigen-binding fragment disclosed herein.

As used herein, the term “derivative” of the polypeptide means a polypeptide chemically modified through conjugation with other chemical moiety, which is different from an insertion, deletion, addition or substitution variant.

As used herein, the term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides are meant to encompass both purified and recombinant polypeptides.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “vector” means any molecule used for delivering a nucleic acid molecule encoding a protein to a host cell, comprising for example, a nucleic acid, a plasmid, a bacteriophage or a virus.

The term “expression vector” means a vector which is suitable for transformation of a host cell and comprises a nucleic acid sequence that is operably connected to an expression vector and regulates the expression of heterologous sequences encoding a targeting protein.

This expression vector may be also operably connected to the coding sequence, and in case of transcription, translation and that an intron is present, it may comprise a sequence regulating RNA splicing or affecting it.

The term “operably connected” means that nucleic acid sequences to be connected are positioned so as to perform a targeting function under an appropriate condition. For example, if the transcription of the coding sequence is affected by the regulatory sequence under an appropriate condition in a vector comprising a coding sequence and a regulatory sequence, it is operably connected.

The term “host cell” means a cell which can express a target gene that is transformed or to be transformed by a targeting nucleic acid sequence. The term includes progeny of the host cell, as long as expressing the targeting gene, regardless of identity of host cell and form and genetic makeup.

The term, “transduction” commonly means movement of a nucleic acid from one bacterium to another bacterium by a bacteriophage. For example, it includes movement of a nucleic acid to a eukaryotic cell using a retrovirus which cannot replicate.

The term “transfection” means that a cell takes a foreign or exogenous DNA, and in this case, DNA is introduced in a cell through a cell membrane. This may refer methods known in the art, for example, Sambrook et al., Molecular Cloning: A Laboratory Manual, 4th ed., Cold Spring Harbor Laboratory Press, Cold Spring Harbor, N.Y (2012), Ausubel et al., Current Protocols in Molecular Biology, Greene Publishing Associates.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, an “antibody”, “antigen-binding region or site” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

In one embodiment, the antibody includes a monoclonal antibody, monospecific antibody, bispecific antibody, trispecific antibody, double antibody, multispecific antibody, multiple antibody, minibody, domain antibody, antibody mimetic (or synthetic antibody), chimeric antibody, humanized antibody or antibody fusion (or antibody conjugate) and fragment thereof, but not limited thereto, and includes various forms of antibodies disclosed herein.

As used herein, the term “antigen” or “immunogen” means a molecule or a part of molecule which for example, an antigen-binding protein (for example, antibody or its immunologically functional antigen-binding fragment) can bind to, and can be used for production of an antibody which can bind to an antigen in an animal. The antigen may comprise one or more of epitopes which can interact with a different antibody or its fragment.

As used herein, the terms “antibody fragment” or “antigen-binding fragment” includes a part of an antibody which lacks some amino acids compared to a full-length chain, but can specifically bind to an antigen. This fragment can be considered as having biological activity, in an aspect that it can specifically bind to a target antigen, or can compete to other antibodies or an antigen-binding fragment to bind a specific epitope. In one aspect, this fragment comprises at least one CDR present in a full-length light chain or heavy chain, and in some embodiments, it comprises a short-chain heavy chain and/or light chain, or its part. This biological active fragment may be produced by a recombinant DNA technique or may be produced for example, by cutting an intact antibody enzymatically or chemically. An immunologically functional immunoglobulin fragment includes Fab, Fab′, F(ab′)₂, scFab, dsFv, Fv, scFv, scFv-Fc, scFab-Fc, diabody, minibody, scAb, dAb, half-IgG or combinations thereof, but not limited thereto. In addition, it may be derived from any mammal including human, mouse, rat, camelid or rabbit, but not limited thereto. The functional part of the antibody such as one or more CDRs described herein may be linked with a secondary protein or small molecular compound by a covalent bond, thereby being used as a target therapeutic agent to a specific target. The term “antibody fragment” includes aptamers, spiegelmers, and diabodies. The term “antibody fragment” also includes any synthetic or genetically engineered protein that acts like an antibody by binding to a specific antigen to form a complex.

Herein, “Fc” region comprises two heavy chain fragments comprising CH2 and CH3 domains of an antibody. These 2 heavy chain fragments are combined each other by hydrophobic interaction of two or more of disulfide bonds and CH3 domain.

Herein, “Fab fragment” consists of 1 light chain and 1 heavy chain comprising CH1 and a variable region only. The heavy chain of Fab molecule cannot form a disulfide bond with other heavy chain molecule.

Herein, “Fab′ fragment” comprises a region between CH1 and CH2 domains of a heavy chain, in addition to Fab fragment, and it can form a disulfide bond between two heavy chains of two molecules of Fab′ fragment, to form a F(ab′)₂ molecule.

Herein, “F(ab′)₂ fragment” comprises two light chains, and two heavy chains comprising a variable region, CH1 and a part of a constant region between CH1 and CH2 domains, as aforementioned, and thereby an intrachain disulfide bond between 2 heavy chains is formed. Thus, the F(ab′)₂ fragment consists of two Fab′ fragments, and the two Fab′ fragments are meeting each other by the disulfide bond between them.

Herein, “Fv region” is an antibody which comprises each variable region of a heavy chain and a light chain, but does not comprise a constant region. scFv is one that Fv is linked by a flexible linker. scFv-Fc is one that Fc is linked to scFv. The minibody is one that CH3 is linked to scFv. The diabody comprises two molecules of scFv. A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art and are described, e.g., in U.S. Pat. No. 5,892,019.

Herein, “short-chain antibody (scAb)” is a single polypeptide chain comprising one variable region of a heavy chain or a light chain constant region in which a heavy chain and light chain variable region is linked by a flexible linker. The short-chain antibody may refer to for example, U.S. Pat. No. 5,260,203, and this is disclosed herein by reference.

Herein, “domain antibody (dAb)” is an immunologically functional immunoglobulin fragment comprising a variable region of heavy chain or a variable region of light chain only. In one embodiment, two or more of VH regions are linked by a covalent bond by a peptide linker, to form a bivalent domain antibody. Two VH regions of this bivalent domain antibody may target the same or different antigen.

Herein, “bivalent antigen-binding protein” or “bivalent antibody” comprises 2 antigen-binding sites. Two antigen-binding sites comprised in this bivalent antibody may have the same antigen specificity or may be a dual-specific antibody binding to different antigens separately.

Herein, “multispecific antigen-binding protein” or “multispecific antibody” is targeting two or more antigens or epitopes, preferably targeting two (i.e. bispecific) or three (e.g. trispecific) antigens or epitopes, more preferably targeting two antigens or epitope.

Herein, “bispecific”, “dual-specific” antigen-binding protein or antibody is a hybrid antigen-binding protein or antibody having 2 different antigen-binding sites. This bispecific antibody is one kind of multispecific antigen-binding protein or multispecific antibody, and it can be produced by known various methods, for example, fusion of hybridoma or linking of Fab′ fragment. For example, Songsivilai and Lachmann, 1990, Clin. Exp. Immunol. 79:315-321; Kostelny et al., 1992, J. Immunol. 148:1547-1553, etc. may be referred. The 2 epitopes different each other to which 2 antigen-binding sites of the bispecific antigen-binding protein or antibody bind may be positioned on the same or different protein target.

Herein, “trispecific” antigen-binding protein or antibody is a hybrid antigen-binding protein or antibody having 3 different antigen-binding sites.

Herein, “multispecific antibody” comprises bispecific antibody and trispecfic antibody, preferably bispecific antibody.

The term “antibody” encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y More specifically, the antigen-binding site is defined by three CDRs on each of the VH and VL chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363: 446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a p-sheet conformation and the CDRs form loops which connect, and in some cases form part of, the 3-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see www.bioinf.org.uk: Dr. Andrew C. R. Martin's Group; “Sequences of Proteins of Immunological Interest,” Kabat, E., et al., U.S. Department of Health and Human Services, (1983); and Chothia and Lesk, J. MoI. Biol., 196: 901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. MoI. Biol. 196: 901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG1 molecule and a hinge region derived from an IgG3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.” Preferably, the antibody binds to an antigen (or epitope) with “high affinity”, namely with a K_{D} of 1×10⁻⁷ M or less, more preferably 5×10⁻⁸ M or less, more preferably 3×10⁻⁸ M or less, more preferably 1×10⁻⁸ M or less, more preferably 25×10⁻⁹ M or less or even more preferably 1×10⁻⁹ M or less.

As used herein, “affinity” is the strength of interaction between an antibody or its antigen-binding fragment and an antigen, and it is determined by properties of the antigen such as size, shape and/or charge of antigen, and CDR sequences of the antibody or antigen-binding fragment. The methods for determining the affinity are known in the art.

Herein, “epitope” is a part of molecule which is bound by an antigen-binding protein or antibody or is recognized by them, and comprise any determining factor which can specifically bind to an antigen-binding protein, such as for example, an antibody or a T-cell receptor. The epitope may be sequential or unsequential, and for example, in a polypeptide sequence, it is not sequential each other, but in an aspect of molecule, like a conformational epitope, it may be an amino acid residue that is bound by one antigen-binding protein, but is not sequential and is positioned away each other. In one embodiment, the epitope comprises a three-dimensional structure similar to an antigen used for antibody production, but it may be a mimetic in an aspect that it can comprise no residue found in the epitope or can comprise some residues only. Commonly, the epitope is a protein, but it may be other kinds of materials such as a nucleic acid. The epitope determining factor may be a chemically active group formed on a surface by a molecule such as an amino acid, a sugar side chain, a phosphoryl group or a sulfonyl group, or may have specific three-dimensional structural properties and/or specific charge properties. Commonly, an antibody which is specific to a specific target antigen recognizes an epitope of a target antigen which is present in a complex of a protein and/or a polymer.

As used herein, the terms “treat” or “treatment” may refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” may refer to any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

The present disclosure provides an anti-PD-L1/anti-B7-H3 multispecific antibody capable to effectively block the interaction between PD-L1 and its receptor PD-1, and suppress the T-cell inhibitory effect of B7-H3 protein. The multispecific antibody may have high binding affinity to both of a PD-L1 protein (e.g., a human PD-L1 protein) and a B7-H3 protein (e.g., a human B7-H3 protein).

The anti-PD-L1/anti-B7-H3 multispecific antibody may comprise an anti-PD-L1 antibody or an antigen-binding fragment thereof as a PD-L1 targeting moiety, which is capable of specifically recognizing and/or binding to a PD-L1 protein, and an anti-B7-H3 antibody or an antigen-binding fragment thereof as a B7-H3 targeting moiety, which is capable of specifically recognizing and/or binding to a B7-H3 protein.

Anti-PD-L1 Antibody

The anti-PD-L1/anti-B7-H3 multispecific antibody may comprise an anti-PD-L1 antibody or an antigen-binding fragment thereof as a PD-L1 targeting moiety. The anti-PD-L1 antibody or antigen-binding fragment thereof may exhibit potent binding and inhibitory activities to PD-L1, and be useful for therapeutic and diagnostics uses.

The PD-L1 protein is a 40 kDa type 1 transmembrane protein. The PD-L1 protein may be a human PD-L1 protein, and the human PD-L1 protein may be selected from the group consisting of proteins represented by GenBank Accession No. NP_001254635.1, NP_001300958.1, NP_054862.1, etc., but may not be limited thereto. The human PD-L1 protein includes an extracellular portion including an N-terminal immunoglobulin V (IgV) domain (amino acids 19-127) and a C-terminal immunoglobulin C (IgC) domain (amino acids 133-225). Unlike pre-existing anti-PD-L1 antibodies, which bind to the IgV domain of PD-L1, thereby disrupting the binding between PD-1 and PD-L1, the anti-PD-L1 antibody or fragment thereof comprised in the multispecific antibody may not bind to an immunoglobulin V (IgV) domain of the PD-L1 protein but bind to the IgC domain of PD-L1, to effectively inhibit PD-L1, thereby improving therapeutic effects.

In particular, the anti-PD-L1 antibody or fragment thereof comprised in the multispecific antibody can specifically bind to an immunoglobulin C (IgC) domain of PD-L1 protein. In the case of human PD-L1 protein, the Ig C domain comprises or consists essentially of amino acid residues 133-225 of full-length of the human PD-L1 protein. More specifically, the anti-PD-L1 antibody or fragment thereof can bind to at least one selected from the amino acid residues Y134, K162, and N183 of human PD-L1 protein. In some embodiments, the anti-PD-L1 antibody or fragment thereof can bind to at least two selected from the amino acid residues Y134, K162, and N183 of human PD-L1 protein. In some embodiments, the anti-PD-L1 antibody or fragment thereof does not bind to an immunoglobulin V (IgV) domain of the PD-L1 protein, wherein the IgV domain consists of amino acid residues 19-127 of human PD-L1 protein.

In an embodiment, the anti-PD-L1 antibody or fragment thereof is capable of specificity to a human PD-L1 protein.

The anti-PD-L1 antibody or fragment thereof may comprise (1) a VH CDR1 having an amino acid sequence selected from the group consisting of SEQ ID NOs: 1 and 294; (2) a VH CDR2 having an amino acid sequence selected from the group consisting of SEQ ID NOs: 2, 3 and 295; (3) a VH CDR3 having an amino acid sequence selected from the group consisting of SEQ ID NOs: 4, 5, 6, 7, 8, 9, 10, 11 and 296; (4) a VL CDR1 having an amino acid sequence selected from the group consisting of SEQ ID NOs: 12, 13, 14 and 297; (5) a VL CDR2 having an amino acid sequence selected from the group consisting of SEQ ID NOs: 15 and 298; and (6) a VL CDR3 having an amino acid sequence selected from the group consisting of SEQ ID NOs: 16, 17, 18, 19 and 299.

The CDR sequences of anti-PD-L to be comprised in heavy chain and light chain variable regions of the antibody or antigen-binding fragment according to one embodiment of the present invention are shown in table 2 below.

TABLE 2

| Name | Sequence | SEQ ID NO: |
|---|---|---|
| VH CDR1 | SYDMS | 1 |
| | SYWMS | 294 |
| VH CDR2 | TISDAGGYIYYSDSVKG | 2 |
| | TISDAGGYIYYRDSVKG | 3 |
| | NIKQDGSEKYYVDSVKG | 295 |
| VH CDR3 | EFGKRYALDY | 4 |
| | ELPWRYALDY | 5 |
| | EFGKRYALDS | 6 |
| | EIFNRYALDY | 7 |
| | ELHFRYALDY | 8 |
| | ELYFRYALDY | 9 |
| | ELLHRYALDY | 10 |
| | ELRGRYALDY | 11 |
| | VALWDDAFDI | 296 |
| VL CDR1 | KASQDVTPAVA | 12 |
| | KAKQDVTPAVA | 13 |
| | KASQDVWPAVA | 14 |
| | RASRGISSWLA | 297 |
| VL CDR2 | STSSRYT | 15 |
| | KASSLES | 298 |
| VL CDR3 | QQHYTTPLT | 16 |
| | MQHYTTPLT | 17 |
| | QQHSTTPLT | 18 |
| | QQHSDAPLT | 19 |
| | QQSSSIPLT | 299 |

In one embodiment, CDRs of each variable region of light chain and CDRs of each variable region of heavy chain disclosed in Table above can be combined freely.

In some embodiments, an antibody or fragment thereof includes no more than one, no more than two, or no more than three of the above substitutions. In some embodiments, the antibody or fragment thereof includes a VH CDR1 of SEQ ID NOs: 1 or 294, a VH CDR2 of SEQ ID NOs: 2, 3 or 295, a VH CDR3 of SEQ ID NOs: 4, 5, 6, 7, 8, 9, 10, 11 or 296, a VL CDR1 of SEQ ID NOs: 12, 13, 14 or 297, a VL CDR2 of SEQ ID NOs: 15 or 298, and a VL CDR3 of SEQ ID NOs: 16, 17, 18, 19 or 299.

For example, the anti-PD-L1 antibody or fragment thereof may comprise a VH CDR1 having an amino acid sequence of SEQ ID NOs: 1 or 294; a VH CDR2 having an amino acid sequence of SEQ ID NO: 2, 3 or 295; a VH CDR3 having an amino acid sequence of SEQ ID NOs: 4, 5 or 296; a VL CDR1 having an amino acid sequence of SEQ ID NOs: 12 or 297; a VL CDR2 having an amino acid sequence of SEQ ID NOs: 15 or 298; and a VL CDR3 having an amino acid sequence of SEQ ID NOs: 16 or 299.

For example, the anti-PD-L1 antibody or fragment thereof may comprise a VH CDR1 having an amino acid sequence of SEQ ID NOs: 1 or 294; a VH CDR2 having an amino acid sequence of SEQ ID NO: 3 or 295; a VH CDR3 having an amino acid sequence of SEQ ID NOs: 5 or 296; a VL CDR1 having an amino acid sequence of SEQ ID NOs: 12 or 297; a VL CDR2 having an amino acid sequence of SEQ ID NOs: 15 or 298; and a VL CDR3 having an amino acid sequence of SEQ ID NOs: 16 or 299.

The back-mutations may be useful for retaining certain characteristics of the anti-PD-L1 antibodies. In some embodiments, the anti-PD-L1 antibodies of the present disclosure, in particular the human or humanized ones, may include one or more of the back-mutations. In some embodiments, the back-mutation (i.e., included amino acid at the specified position) in a heavy chain variable region (VH) is one or more selected from (a) Ser at position 44, (b) Ala at position 49, (c) Ala at position 53, (d) Ile at position 91, (e) Glu at position 1, (f) Val at position 37, (g) Thr at position 40, (h) Val at position 53, (i) Glu at position 54, (j) Asn at position 77, (k) Arg at position 94, and (1) Thr at position 108, of the heavy chain variable region, according to Kabat numbering, and combinations thereof. In some embodiments, the VH back-mutations are selected from (a) Ser at position 44, (b) Ala at position 49, (c) Ala at position 53, and/or (d) Ile at position 91, of the heavy chain variable region, according to Kabat numbering, and combinations thereof.

In some embodiments, the back-mutation in a light chain variable region (VL) is one or more selected from (a) Ser at position 22, (b) Gln at position 42, (c) Ser at position 43, (d) Asp at position 60, and (e) Thr at position 63, of the light chain variable region, according to Kabat numbering, and combinations thereof.

In some embodiments, the anti-PD-L1 antibody or fragment thereof comprises a heavy chain constant region, a light chain constant region, an Fc region, or the combination thereof. In some embodiments, the light chain constant region may be a kappa or lambda chain constant region. In some embodiments, the antibody is of an isotype of IgG, IgM, IgA, IgE or IgD, for example, human IgG, human IgM, human IgA, human IgE, or human IgD. In some embodiments, the isotype may be IgG, for example human IgG, such as, IgG1, IgG2, IgG3, or IgG4. In some embodiments, the fragment (antigen-binding fragment of the anti-PD-L1 antibody) may be any fragment comprising heavy chain CDRs and/or light chain CDRs of the antibody, and for example, it may be selected from, but not limited to, the group consisting of Fab, Fab′, F(ab′)₂, Fd (comprising a heavy chain variable region and a CH1 domain), Fv (a heavy chain variable region and/or a light chain variable region), single-chain Fv (scFv; comprising or consisting essentially of a heavy chain variable region and a light chain variable region, in any order, and a peptide linker between the heavy chain variable region and the light chain variable region), single-chain antibodies, disulfide-linked Fvs (sdFv), scFab (single chain Fab), scFab-Fc (comprising scFab and Fc region), half-IgG (comprising one light chain and one heavy chain) and the like.

Without limitation, the anti-PD-L1 antibody or fragment thereof is a chimeric antibody, a humanized antibody, or a fully human antibody. In one aspect, antibody or fragment thereof is not naturally occurring, or chemically or recombinantly synthesized.

The binding of an antibody of the disclosure to PD-L1 can be assessed using one or more techniques well established in the art. For example, in a preferred embodiment, an antibody can be tested by a flow cytometry assay in which the antibody is reacted with a cell line that expresses human PD-L1, such as CHO cells that have been transfected to express PD-L1, e.g., human PD-L1, or monkey PD-L1, e.g., rhesus or cynomolgus monkey or mouse PD-L1 on their cell surface. Other suitable cells for use in flow cytometry assays include anti-CD3-stimulated CD4+ activated T cells, which express native PD-L1. Still other suitable binding assays include ELISA assays, for example using a recombinant PD-L1 protein. Additionally, or alternatively, the binding of the antibody, including the binding kinetics (e.g., K_{D} value) can be tested in Biacore analysis. Preferred binding affinities of an antibody of the disclosure include those with a dissociation constant or K_{D} of 4.25×10⁻⁹ M or less.

Given that each of these antibodies can bind to PD-L1 such as human PD-L1, the CDR sequences or VH and VL sequences can be “mixed and matched” to create other anti-PD-L1 binding molecules of the disclosure. Preferably, when the CDR sequences or VH and VL chains are mixed and matched, for example, a VH sequence from a particular VH/VL pairing is replaced with a structurally similar VH sequence. Likewise, preferably a VL sequence from a particular VH/VL pairing is replaced with a structurally similar VL sequence.

Anti-B7-H3 Antibody

The anti-PD-L1/anti-B7-H3 multispecific antibody may comprise an anti-B7-H3 antibody or an antigen-binding fragment thereof as a B7-H3 targeting moiety. The anti-B7-H3 antibody or antigen-binding fragment thereof may specifically recognize a human B7-H3, and shows cross-reactivity to a monkey and mouse B7-H3. The anti-B7-H3 antibody or antigen-binding fragment thereof can inhibit or block a B7-H3 immune checkpoint, thereby reactivating a T cell in which the activity is degraded or inhibited by the B7-H3 immune checkpoint. Thus, the antibody or antigen-binding fragment may be usefully used for reactivation of a T cell inhibited by the B7-H3 immune checkpoint and treatment of various diseases requiring the reactivation, through such B7-H3 immune checkpoint inhibition.

The B7-H3 (B7 Homolog 3, CD276), that is recognized by the antibody or antigen-binding fragment thereof described herein, may refer to a transmembrane protein of a B7 family belonging to an immunoglobulin (Ig) superfamily, and comprises an extracellular domain, a transmembrane domain and an intracellular domain. The B7-H3 which the antibody recognizes may be an extracellular domain which is present in a cell membrane or is not present in a cell membrane. The B7-H3 which the antibody recognizes may be an extracellular domain which is present in a cell membrane or is not present in a cell membrane. The human protein of B7-H3 consists of 534 amino acids, and it is disclosed as NCBI Reference Sequence: NP_001019907.1. Unless apparent from the context used herein, the B7-H3 refers to a human B7-H3, but the antibody has the binding capacity to monkey and mouse B7-H3 specifically. The monkey B7-H3 protein consists of 534 amino acids, and is disclosed as NCBI Reference Sequence: XP_005560056.1. The mouse B7-H3 protein consists of 316 amino acids, and is disclosed as NCBI Reference Sequence: NP_598744.1.

The anti-B7-H3 antibody disclosed herein, is a polypeptide comprising one or more of complementary determining regions or sites (CDR), as disclosed herein.

The antibody specifically binds to a human, monkey and mouse-derived B7-H3 extracellular domain, and it can specifically bind to an isolated form of extracellular domain or an extracellular domain of B7-H3 expressed on a cell surface.

The anti-B7-H3 antibody or fragment thereof may comprise (1) a VH CDR1 having an amino acid sequence selected from the group consisting of SEQ ID NOs: 20, 21, 22 and 23; (2) a VH CDR2 having an amino acid sequence selected from the group consisting of SEQ ID NOs: 24, 25, 26, 27, 28 and 29; and (3) VH CDR3 having an amino acid sequence selected from the group consisting of SEQ ID NOs: 30, 31, 32, 33 and 34; (4) a VL CDR1 having an amino acid sequence selected from the group consisting of SEQ ID NOs: 35, 36, 37, 38 and 39; (5) a VL CDR2 having an amino acid sequence selected from the group consisting of SEQ ID NOs: 40, 41, 42, 43, 44 and 45; and (6) a VL CDR3 having an amino acid sequence selected from the group consisting of SEQ ID NOs:46, 47, 48, 49, and 50.

The CDR sequences of amino acid of anti-B7-H3 to be comprised in heavy chain and light chain variable regions of the antibody or antigen-binding fragment according to one embodiment of the present invention are disclosed in Table below.

TABLE 3

| Name | Sequence | SEQ ID NO: |
|---|---|---|
| VH CDR1 | DYAMS | 20 |
| | GYYMS | 21 |
| | SYSMS | 22 |
| | SYGMS | 23 |
| VH CDR2 | SISSGSGSIYYADSVKG | 24 |
| | LISPSSGSIYYADSVKG | 25 |
| | GIYSDGSNTYYADSVKG | 26 |
| | GISPGGSNTYYADSVKG | 27 |
| | GIYSGGSSKYYADSVKG | 28 |
| | GIYSDASNTYYADSVKG | 29 |
| VH CDR3 | NLIPLDY | 30 |
| | GLTKFDY | 31 |
| | MLHRFDY | 32 |
| | DAWIARLLLFDY | 33 |
| | NRLRFDY | 34 |
| VL CDR1 | SGSSSNIGSNAVS | 35 |
| | TGSSSNIGSNDVS | 36 |
| | SGSSSNIGSNSVT | 37 |
| | SGSSSNIGSNAVT | 38 |
| | TGSSSNIGSNSVT | 39 |
| VL CDR2 | YNSHRPS | 40 |
| | ANSHRPS | 41 |
| | ADSQRPS | 42 |
| | YNNKRPS | 43 |
| | SDSHRPS | 44 |
| | ADVQRPS | 45 |
| VL CDR3 | GSWDASLNAYV | 46 |
| | GSWDDSLSGYV | 47 |
| | GTWDSSLNAYV | 48 |
| | GTWDDSLSGYV | 49 |
| | GTWDASLNAYV | 50 |

In some embodiments, an antibody or fragment thereof includes no more than one, no more than two, or no more than three of the above substitutions. In some embodiments, the antibody or fragment thereof includes a VH CDR1 of SEQ ID NOs: 20 or 21, a VH CDR2 of SEQ ID NOs: 24 or 25, a VH CDR3 of SEQ ID NOs: 30 or 31, a VL CDR1 of SEQ ID NOs: 35 or 36, a VL CDR2 of SEQ ID NOs: 40 or 41, and a VL CDR3 of SEQ ID NOs: 46 or 47.

For example, the anti-B7-H3 antibody or fragment thereof may comprise a VH CDR1 having an amino acid sequence of SEQ ID NOs: 20 or 21; a VH CDR2 having an amino acid sequence of SEQ ID NO: 24 or 25; a VH CDR3 having an amino acid sequence of SEQ ID NOs: 30 or 31; a VL CDR1 having an amino acid sequence of SEQ ID NOs: 35 or 36; a VL CDR2 having an amino acid sequence of SEQ ID NOs: 40 or 41; and a VL CDR3 having an amino acid sequence of SEQ ID NOs: 46 or 47.

In one embodiment, CDRs of each variable region of light chain and CDRs of each variable region of heavy chain disclosed in Table above can be combined freely.

In one embodiment, heavy chain and light chain variable regions of the antibody or antigen-binding fragment comprising the light chain and heavy chain CDR sequences may be exemplified in the following Table below.

TABLE 4

| | SEQ |
|---|---|
| Heavy Chain Variable Region (VH) Sequence | ID NO |
| EVQLLESGGGLVQPGGSLRLSCAASGFTFSDYAMSWVRQAPGKGLEWVSSISSGSGSIYYA | 51 |
| DSVKGRFTISRDNSKNTLYLQMNSLRAEDTAVYYCAKNLIPLDYWGQGTLVTVSS | |
| EVQLLESGGGLVQPGGSLRLSCAASGFTFSGYYMSWVRQAPGKGLEWVSLISPSSGSIYYA | 52 |
| DSVKGRFTISRDNSKNTLYLQMNSLRAEDTAVYYCAKGLTKFDYWGQGTLVTVSS | |
| EVQLLESGGGLVQPGGSLRLSCAASGFTFSSYSMSWVRQAPGKGLEWVSGIYSDGSNTYY | 53 |
| ADSVKGRFTISRDNSKNTLYLQMNSLRAEDTAVYYCAKMLHRFDYWGQGTLVTVSS | |
| EVQLLESGGGLVQPGGSLRLSCAASGFTFSDYAMSWVRQAPGKGLEWVSGISPGGSNTYY | 54 |
| ADSVKGRFTISRDNSKNTLYLQMNSLRAEDTAVYYCAKDAWIARLLLFDYWGQGTLVTV | |
| SS | |
| EVQLLESGGGLVQPGGSLRLSCAASGFTFSSYGMSWVRQAPGKGLEWVSGIYSGGSSKYY | 55 |
| ADSVKGRFTISRDNSKNTLYLQMNSLRAEDTAVYYCAKNRLRFDYWGQGTLVTVSS | |
| EVQLLESGGGLVQPGGSLRLSCAASGFTFSSYSMSWVRQAPGKGLEWVSGIYSDASNTYY | 56 |
| ADSVKGRFTISRDNSKNTLYLQMNSLRAEDTAVYYCAKMLHRFDYWGQGTLVTVSS | |
| | SEQ |
| Light Chain Variable Region (VL) Sequence | ID NO |
| QSVLTQPPSASGTPGQRVTISCSGSSSNIGSNAVSWYQQLPGTAPKLLIYYNSHRPSGVPDRF | 57 |
| SGSKSGTSASLAISGLRSEDEADYYCGSWDASLNAYVFGGGTKLTVL | |
| QSVLTQPPSASGTPGQRVTISCTGSSSNIGSNDVSWYQQLPGTAPKLLIYANSHRPSGVPDR | 58 |
| FSGSKSGTSASLAISGLRSEDEADYYCGSWDDSLSGYVFGGGTKLTVL | |
| QSVLTQPPSASGTPGQRVTISCSGSSSNIGSNSVTWYQQLPGTAPKLLIYADSQRPSGVPDRF | 59 |
| SGSKSGTSASLAISGLRSEDEADYYCGTWDSSLNAYVFGGGTKLTVL | |
| QSVLTQPPSASGTPGQRVTISCSGSSSNIGSNAVTWYQQLPGTAPKLLIYYNNKRPSGVPDR | 60 |
| FSGSKSGTSASLAISGLRSEDEADYYCGTWDDSLSGYVFGGGTKLTVL | |
| QSVLTQPPSASGTPGQRVTISCTGSSSNIGSNSVTWYQQLPGTAPKLLIYSDSHRPSGVPDRF | 61 |
| SGSKSGTSASLAISGLRSEDEADYYCGTWDASLNAYVFGGGTKLTVL | |
| QSVLTQPPSASGTPGQRVTISCSGSSSNIGSNSVTWYQQLPGTAPKLLIYADVQRPSGVPDR | 62 |
| FSGSKSGTSASLAISGLRSEDEADYYCGTWDSSLNAYVFGGGTKLTVL | |

In other embodiment, the variable regions of heavy chain and light chain disclosed in Table above can be combined freely for preparation of various forms of antibodies, and for example, a single antibody such as scFv, or domain antibody can be formed.

Each of heavy chain and light chain variable regions disclosed herein may bind to targeting various heavy chain and light chain constant regions to form heavy chain and light chain of an intact antibody, respectively. In addition, each of heavy chain and light chain sequences bound to constant regions like this may be also combined to form an intact antibody structure.

Any variable region of heavy chain and light chain of the antibody may be linked to at least a part of constant regions. The constant regions may be selected according to whether antibody-dependent cell-mediated cytotoxicity, antibody-dependent cell phagocytosis and/or complement-dependent cytotoxicity, etc. is required. For example, Human isotype IgG1 and IgG3 have complement-dependent cytotoxicity, and human isotype IgG2 and IgG4 do not have the cytotoxicity. Human IgG1 and IgG3 also induce a cell-mediated effector function stronger than human IgG2 and IgG4. For example, the heavy chain variable region may bind to a constant region of IgG, such as IgG1, IgG2, IgG2a, IgG2b, IgG3 and IgG4, and the light chain variable region may bind to a kappa or lambda constant region. For the constant region, one appropriate as desired can be used, and for example, a human or mouse-derived one can be used. In one embodiment, a human heavy chain constant region IgG1 is used, and this may be represented by the sequence of SEQ ID NO: 157. In other embodiment, as the light chain constant region, a human lambda region is used, and this may be represented by SEQ ID NO: 161.

Any variable region disclosed herein may be bound to a constant region, thereby forming heavy chain and light chain sequences. In one embodiment, the heavy chain variable region disclosed herein may be bound to a human IgG1 constant region, to form a heavy chain (full-length) comprising or consisting essentially of an amino acid sequence selected from SEQ ID NOs: 282 to 286, and 292. In other embodiment, the light chain variable region disclosed herein may be bound to a human lambda constant region, to form and the light chain (full-length) comprising or consisting essentially of an amino acid sequence selected from SEQ ID NOs: 287 to 291, and 293. The light chain and heavy chain can be combined as various combinations, thereby forming an intact antibody consisting of two light chains and two heavy chains.

In other embodiment, the antibody may comprise or consist essentially of a combination of a heavy chain and a light chain, which are represented by the following sequence: SEQ ID NOs: 282 and 287; SEQ ID NOs: 283 and 288; SEQ ID NOs: 284 and 289; SEQ ID NOs: 285 and 290; SEQ ID NOs: 286 and 291; SEQ ID NOs: 292 and 289, or SEQ ID NOs: 292 and 293.

However, such constant region sequences to be combined with the variable regions disclosed herein are exemplary, and those skilled in the art will know that other constant regions including IgG1 heavy chain constant region, IgG3 or IgG4 heavy chain constant region, any kappa or lambda light chain constant region, constant regions modified for stability, expression, manufacturability or other targeting properties, etc. may be used.

In some embodiments, the antigen-binding fragment of the anti-B7-H3 antibody may be any fragment comprising heavy chain CDRs and/or light chain CDRs of the antibody, and for example, it may be selected from, but not limited to, the group consisting of Fab, Fab′, F(ab′)₂, Fd (comprising a heavy chain variable region and a CH1 domain), Fv (a heavy chain variable region and/or a light chain variable region), single-chain Fv (scFv; comprising or consisting essentially of a heavy chain variable region and a light chain variable region, in any order, and a peptide linker between the heavy chain variable region and the light chain variable region), single-chain antibodies, disulfide-linked Fvs (sdFv), scFab (single chain Fab), scFab-Fc (comprising scFab and Fc region), half-IgG (comprising one light chain and one heavy chain) and the like.

The present invention comprises one or more amino acid sequences having substantial sequence identity with one or more amino acid sequences disclosed herein. The substantial identity means maintaining the effect disclosed herein in which the sequence variation is present. In one embodiment, it has about 90%, 95%, or 99% identity to the heavy chain variable regions disclosed in Table 4. In other embodiment, it has about 90%, 95%, or 99% identity to the light chain variable regions disclosed in Table 4. For example, in case of variant showing 90%, 95%, or 99% identity to the antibody or antigen-binding fragment disclosed herein, any variation is occurred in a frame of variable regions than CDRs.

Anti-PD-L1/Anti-B7-H3 Multispecific Antibody

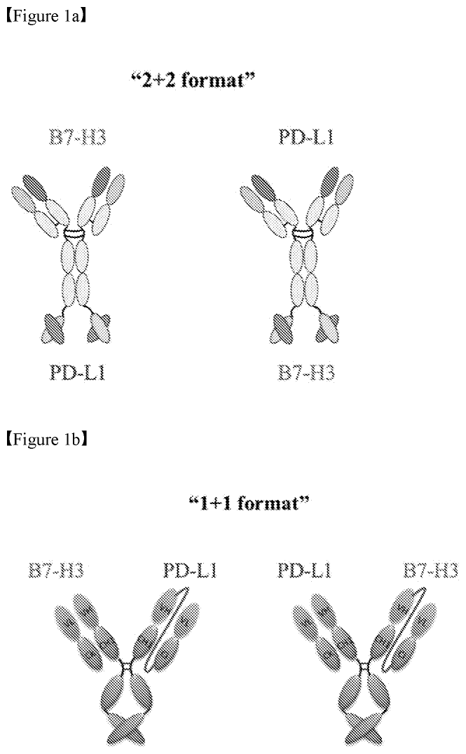

In an embodiment, in the multispecific antibody comprising the PD-L1 targeting moiety and the B7-H3 targeting moiety, one of the PD-L1 targeting moiety and the B7-H3 targeting moiety can be a full-length antibody, and the other can be an antigen-binding fragment (e.g., scFv) comprising heavy chain CDRs, light chain CDRs, or a combination thereof. The full-length antibody targeting one of PD-L1 and B7-H3 proteins, and the antigen-binding fragment targeting the other protein may be chemically linked (e.g., covalently linked) directly or via a peptide linker. The antigen-binding fragment (e.g., scFv) may be linked directly or via a peptide linker to N-terminus of the full-length antibody (e.g., N-terminus of a light chain or a heavy chain of the full-length antibody), C-terminus of the full-length antibody (e.g., C-terminus of a heavy chain (or Fc or CH3 domain) of the full-length antibody), or both thereof (see FIG. 1a).

In an embodiment, the multispecific antibody may comprise a full-length anti-PD-L1 antibody, an antigen-binding fragment (e.g., scFv) of an anti-B7-H3 antibody, and a peptide linker therebetween. In other embodiment, the multispecific antibody may comprise a full-length anti-B7-H3 antibody, an antigen-binding fragment (e.g., scFv) of an anti-PD-L1 antibody, and a peptide linker therebetween (See FIG. 1a).

In an embodiment, the scFv contained in the multispecific antibody may comprise a heavy chain variable region and a light chain variable region in any order. For example, the scFv contained in the multispecific antibody may comprise a heavy chain variable region and a light chain variable, in a direction from N-terminus to C-terminus, and optionally a peptide linker therebetween, or alternatively, the scFv contained in the multispecific antibody may comprise a light chain variable region and a heavy chain variable, in a direction from N-terminus to C-terminus, and optionally a peptide linker therebetween.

The scFv may comprise additional modification (mutation of amino acids at VL100 and VH44 of scFv to cysteine) to generate disulfide bridge fusing VL100-VH44 to variable light chain and variable heavy chain, respectively, for stabilizing scFv.

The anti-PD-L1/anti-B7-H3 multispecific antibody of the present disclosure may be an IgG X scFv form antibody, which can be also referred to as “2+2 format antibody.” The IgG X scFv form antibody may have a structure that scFv is linked to C-terminus of each Fc region of heavy chains of a full-length IgG antibody via linker (see FIG. 1a), and comprise Heavy Component and Light Component.

As used herein, “Heavy Component” means a component of anti-PD-L1/anti-B7-H3 multispecific antibody of an embodiment of the present disclosure, which comprises i) a heavy chain of anti-PD-L1 antibody, and a variable heavy chain and a variable light chain of anti-B7-H3 antibody, or ii) a heavy chain of anti-B7-H3 antibody, and a variable heavy chain and a variable light chain of anti-PD-L1 antibody.

As used herein, “Light Component” means a component of anti-PD-L1/anti-B7-H3 multispecific antibody of an embodiment of the present disclosure, which comprises i) a light chain of anti-PD-L1 antibody if the Heavy Component comprises a heavy chain of anti-PD-L1 antibody, or ii) a light chain of anti-B7-H3 if the Heavy Component comprises a heavy chain of anti-B7-H3 antibody.

In another embodiment, in the multispecific antibody comprising the PD-L1 targeting moiety and the B7-H3 targeting moiety, neither the PD-L1 targeting moiety nor the B7-H3 targeting moiety is a full-length antibody. In this case, any one of the PD-L1 targeting moiety and the B7-H3 targeting moiety may comprise one heavy chain (HC) and one light chain (LC), and the other one may comprise scFab-Fc. “scFab-Fc” means the structure comprising scFab and Fc linked thereto. In this structure, the scFab may be chemically linked (e.g., covalently linked) to Fc region directly or via a peptide linker.

In an embodiment, the multispecific antibody may comprise HC+LC (which can be also referred to a half-IgG) of anti-PD-L1 antibody and scFab-Fc of anti-B7-H3 antibody. In other embodiment, the multispecific antibody may comprise HC+LC of anti-B7-H3 antibody and scFab-Fc of anti-PD-L1 antibody. These types of multispecific antibodies may be referred to as (HC+LC) X scFab-Fc form antibody, or also “1+1 format antibody.”

In other word, the (HC+LC) X scFab-Fc form antibody may have a structure that any one arm (VH, CH1 and a light chain) of an IgG antibody is substituted with scFab (VL-CL-VH-CH1 in the order from the N-terminus to the C-terminus) (see FIG. 1b). The C-terminus of the scFab may be linked to the N-terminus of Fc chain via linker. In another word, the (HC+LC) X scFab-Fc form antibody may comprise a half-IgG and a scFab-Fc.

In an embodiment, the 1+1 format antibody may further comprise scFv at the C-terminus of each Fe to form a trispecific antibody. The trispecific antibody may have a structure that a scFv is linked to a 1+1 format multispecific antibody via a linker. The scFv may bind to a target other than PD-L1 or B7-H3. For example, the scFv may bind to human 4-1BB protein.

The term “4-1BB” refers to CD137, or TNFRSF9 (TNF Receptor 25 Superfamily Member 9), is a member of TNF-receptor superfamily (TNFRSF) and is a co-stimulatory molecule which is expressed following the activation of immune cells, both innate and adaptive immune cells. As used herein, 4-1BB may be originated from a mammal, for example, Homo sapiens (human) (NCBI Accession No. NP_001552).

The use of a peptide linker for the multispecific antibody may lead to a high purity of the antibody.

As used herein, the term “peptide linker” may be those including any amino acids of 1 to 100, particularly 2 to 50, and any kinds of amino acids may be included without any restrictions. The peptide linker may include for example, Gly, Asn and/or Ser residues, and also include neutral amino acids such as Thr and/or Ala. Amino acid sequences suitable for the peptide linker may be those known in the relevant art. Meanwhile, a length of the peptide linker may be variously determined within such a limit that the functions of the fusion protein will not be affected. For instance, the peptide linker may be formed by including a total of about 1 to about 100, about 2 to about 50, or about 5 to about 25 of one or more selected from the group consisting of Gly, Asn, Ser, Thr, and Ala. In one embodiment, the peptide linker may be represented as (GmSl)n (m, l, and n, are independently an integer of about 1 to about 70, particularly an integer of about 1 to about 64). For example, the examples of the peptide liners are summarized as follows:

| | Examples | | | |
|---|---|---|---|---|
| Linker | | Linker | | |
| Function | Fusion Protein | Type | Sequence | Ref. |
| Increase | scFv | flexible | (GGGGS)3 | [46] |
| Stablility/ | G-CSF-Tf | flexible | (GGGGS)3 | [20] |
| Folding | HBsAg preS1 | flexible | (GGGGS)3 | [85] |
| | Myc- [symbol] | flexible | (Gly) [symbol] | [30] |
| | albumin-ANF | flexible | (Gly) [symbol] | [31] |
| | virus [symbol] protein | rigid | (EAAAK) [symbol] | [50] |
| | [symbol] | rigid | (EAAAK) [symbol] ( [symbol] ) | [52] |
| Increase | hGH-Tf and Tf-hGH | rigid | A(EAAAK) [symbol] ALEA(EAAAK) [symbol] A | [18] |
| expression | g-csf-tF AND | rigid | A(EAAAK) [symbol] ALEA(EAAAK) [symbol] A | [18] |
| Improve | G-CSF-Tf | flexible | (GGGGS) [symbol] | [20] |
| biological | G-CSF-Tf | rigid | A(EAAAK) [symbol] ALEA(EAAAK) [symbol] A | [20] |
| activity | hGH-Tf | rigid | A(EAAAK) [symbol] ALEA(EAAAK) [symbol] A | [40] |
| | HSA-EFN- [symbol] 2b | flexible | GGGGS | [17] |
| | HSA-EFN- [symbol] 2b | rigid | PAPAP | [17] |
| | HSA-EFN- [symbol] 2b | rigid | AEAAAKEAAAKA | [17] |
| | PGA-r [symbol] | flexible | (GGGGS) [symbol] ( [symbol] ) | [55] |
| | [symbol] | rigid | (Ala-Pro) [symbol] (10 - 34 aa) | [54] |
| | GSF-S-S-Tf | cleavable | disulfide | [39] |
| | IFN- [symbol] 2b-HSA | cleavable | disulfide | [42] |
| Enable | FIX-albumin | cleavable | VSQTSKLTR AETVFPDV [symbol] | [59] |
| targeting | LAP-IFN- | cleavable | PLG LWA [symbol] | [64] |
| | MazE-MzaF | cleavable | RVL AEA; EDVVCC SMSY; | [68] |
| | | | GG [symbol] EGR GS [symbol] | |
| | Immunotoxins | cleavable | TRHRQPR GWF; | [72] |
| | | | AGNRVRR SVG; | |
| | | | RRRRRRR R R^{d} | |
| | Immunotoxin | cleavable | GFLG [symbol] | [77] |
| Alter PK | G-CSE-tf and hGH-Tf | dipeptide | LE | [79] |
| | | rigid | A(EAAAK) [symbol] ALEA(EAAAK) [symbol] A | |
| | | cleavable | Disulfide | |

[symbol]

indicates data missing or illegible when filed

Heterodimerization of the two heavy chains in the multispecific antibody (either 2+2 format or 1+1 format) can be facilitated by application of the knobs-into-hole technology. For example, the knob mutation (T366W) was introduced into the CH3 domain of an heavy chain, and three mutations to form a hole (T366S, L368A, and Y407V) were introduced into the CH3 domain of the other heavy chain.

In another embodiment, both of the PD-L1 targeting moiety and the B7-H3 targeting moiety may be a full-length antibody or an antigen-binding fragment comprising heavy chain CDRs, light chain CDRs, or a combination thereof.

In an embodiment, the full-length antibody may be in a full-length immunoglobulin form (e.g., IgG, IgM, IgA, IgE or IgD, such as, human IgG, human IgM, human IgA, human IgE, or human IgD), and the antigen-binding fragment may be selected from the group consisting of Fab, Fab′, F(ab′)2, Fd, Fv, scFv, single-chain antibodies, sdFv, scFab (single chain Fab), scFab-Fc (comprising scFab and Fc region), half-IgG (comprising one light chain and one heavy chain) and the like, as described above. For example, the full-length antibody may be in a full-length human IgG (human IgG1, human IgG2, human IgG3, or human IgG4) form, and the antigen-binding fragment may be scFv.

For example, an antibody described herein may comprise a flexible linker sequence, or may be modified to add a functional moiety (e.g., PEG, a drug, a toxin, or a label).

Antibodies or variants described herein may comprise derivatives that are modified, e.g., by the covalent attachment of any type of molecule to the antibody such that covalent attachment does not prevent the antibody from binding to the antigen (e.g., an epitope). For example, but not by way of limitation, the antibodies can be modified, e.g., by at least one selected from the group consisting of glycosylation, acetylation, pegylation, phosphorylation, phosphorylation, amidation, derivatization by known protecting/blocking groups, proteolytic cleavage, linkage to a cellular ligand or other protein, and the like. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

The binding specificity and/or affinity of the multispecific antibody to each target protein can be determined by any conventional assay, for example, in vitro assays such as immunoprecipitation, radioimmunoassay (RIA), or enzyme-linked immunoabsorbent assay (ELISA), but not be limited thereto.

Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen-binding.

Additionally, standard techniques known to those of skill in the art can be used to introduce mutations in the nucleotide sequence encoding an antibody of the present disclosure.

Therapeutic Use of the Multispecific Antibody

The multispecific antibody provided herein is capable of simultaneously blocking the activities of PD-L1 and B7-H3, thereby exhibiting improved effects in immunotherapies and/or cancer therapies, for example, by activating immune response. Given the ability of the multispecific antibodies of the disclosure to inhibit the binding of PD-L1 to PD-1 molecules and to stimulate antigen-specific T cell responses, the disclosure also provides a composition or in vitro and in vivo methods of using the antibodies of the disclosure to stimulate, enhance or upregulate antigen-specific T cell responses.

An embodiment provides a pharmaceutical composition comprising the multispecific antibody as described above. The pharmaceutical composition may further comprise a pharmaceutically acceptable carrier. The pharmaceutical composition may be used for stimulating an immune response (e.g., an antigen-specific T cell response), and/or treating and/or preventing a disease associated with PD-L1, B7-H3, or both thereof.

Another embodiment provides a method of stimulating an immune response (e.g., an antigen-specific T cell response), and/or treating and/or preventing a disease associated with PD-L1, B7-H3, or both thereof, in a subject in need thereof, comprising administering to the subject a pharmaceutically effective amount of the multispecific antibody or the pharmaceutical composition. The method may further step of identifying the subject in need of treating and/or preventing a disease associated with PD-L1, B7-H3, or both thereof, prior to the administering step.

The disease associated with PD-L1, B7-H3, or both thereof may be selected from cancers (or tumors), infectious diseases, autoimmune reactions, nervous system disorders, and the like.

In an embodiment, the subject may be selected from mammals including humans, for example, a mammal (e.g., a human) suffering from a cancer mammalian cells. In other embodiment, the subject may be a cell separated (isolated) from a mammal, for example, a mammal suffering from the disease selected from cancers infectious diseases, autoimmune reactions, nervous system disorders, and the like (e.g., a cancer cell or a cell separated (isolated) from an infectious region in the mammal, or a T cell, such as a tumor-infiltrating T lymphocyte, a CD4+ T cell, a CD8+ T cell, or the combination thereof).

Another embodiment provides a use of the multispecific antibody or the pharmaceutical composition in treating and/or preventing a cancer. Another embodiment provides a use of the multispecific antibody in preparing a pharmaceutical composition for treating and/or preventing a cancer.

In the pharmaceutical compositions, methods and/or uses provided herein, the disease associated with PD-L1, B7-H3, or both thereof may be one associated with activation (e.g., abnormal activation or over-activation) and/or overproduction (overexpression) of PD-L1, B7-H3, or both thereof. For example, the disease may be a cancer.

The cancer may be a solid cancer or blood cancer, preferably a solid cancer including, but not limited to, breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer and adrenal cancer.

The administration of the multispecific antibody may be conducted by one or more techniques well established in the art.

A “therapeutically effective dosage” of the antibody of the disclosure preferably results in a decrease in severity of disease symptoms, an increase infrequency and duration of disease symptom-free periods, or a prevention of impairment or disability due to the disease affliction. For example, for the treatment of tumor bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human or can be another mammal.

The pharmaceutical compositions may comprise an effective amount of the multispecific antibody, and an acceptable carrier. In some embodiments, the composition further includes a second anticancer agent (e.g., an immune checkpoint inhibitor).

In a specific embodiment, the term “pharmaceutically acceptable” may refer to approved by a regulatory agency of the Federal or a state government or listed in the U.S.

Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The composition comprising the antibody or the antigen-binding fragment thereof of the present disclosure may further comprise a pharmaceutically acceptable carrier. The pharmaceutically acceptable carrier is the one conventionally used in preparing a formulation.

Diagnostic Use of the Multispecific Antibody

Over-expression and/or over-activation of PD-L1 and/or B7-H3 is observed in a biological sample (e.g., cells, tissues, blood, serum, etc.) from a patient suffering from a certain cancer (for example, tumor cell), and/or patients having PD-L1- and/or B7-H3-over-expressing cells are likely responsive to treatments with the multispecific antibody. Accordingly, the multispecific antibody of the present disclosure can also be used for diagnostic and prognostic purposes.

An embodiment provides a pharmaceutical composition for diagnosing a disease associated with PD-L1, B7-H3, or both thereof, the composition comprising the multispecific antibody. In another embodiment, provided is a use of the multispecific antibody for diagnosing a disease associated with PD-L1, B7-H3, or both thereof.

Polynucleotides Encoding the Antibodies and Methods of Preparing the Antibodies

An embodiment provides a polynucleotide encoding the multispecific antibody. In particular, an embodiment provides a polynucleotide encoding a heavy chain of the multispecific antibody in an IgG X scFv form. Other embodiment provides a polynucleotide encoding a light chain of the multispecific antibody in the IgG X scFv form. The IgG X scFv form may refer to a kind of a multispecific antibody comprising a full-length IgG antibody targeting (binding to) one of PD-L1 and B7-H3 proteins and a scFv fragment targeting (binding to) the other one, wherein the scFv is linked to a C-terminus and/or N-terminus of the full-length IgG antibody directly (without a peptide linker) or via a peptide linker.

In an embodiment, when the multispecific antibody in an IgG X scFv form comprises a full-length IgG antibody against PD-L1 and a scFv fragment against B7-H3, the polynucleotide encoding a heavy chain of the multispecific antibody may encode a heavy chain of the full-length IgG antibody against PD-L1 and a scFv fragment against B7-H3 that is linked to a C-terminus and/or N-terminus of the full-length IgG antibody directly or via a peptide linker; and the polynucleotide encoding a light chain of the multispecific antibody may encode a light chain of the full-length IgG antibody against PD-L1.

In another embodiment, when the multispecific antibody in an IgG X scFv form comprises a full-length IgG antibody against B7-H3 and a scFv fragment against PD-L1, the polynucleotide encoding a heavy chain of the multispecific antibody may encode a heavy chain of the full-length IgG antibody against B7-H3 and a scFv fragment against PD-L1 that is linked to a C-terminus and/or N-terminus of the full-length IgG antibody directly or via a peptide linker; and the polynucleotide encoding a light chain of the multispecific antibody may encode a light chain of the full-length IgG antibody against B7-H3.

In particular, an embodiment provides a polynucleotide encoding (HC+LC) X scFab-Fc form antibody (1+1 format antibody). The (HC+LC) X scFab-Fc form antibody may have a structure that any one arm (VH, CH1 and a light chain) of an IgG antibody is substituted with scFab (VL-CL-VH-CH1 in the order from the N-terminus to the C-terminus). The C-terminus of the scFab may be linked to the N-terminus of Fc chain via linker. “scFab-Fc” means the structure comprising scFab and Fc linked thereto. In other word, the (HC+LC) X scFab-Fc form antibody may comprise half-IgG (one heavy chain and one light chain) and scFab-Fc.

In particular, an embodiment provides a polynucleotide encoding trispecific antibody. The trispecific antibody may have a structure that a scFv is linked to a 1+1 format multispecific antibody via a linker. The scFv may bind to a target other than PD-L1 or B7-H3. For example, the scFv may bind to human 4-1BB protein.

Another embodiment provides a recombinant vector comprising the polynucleotide encoding a heavy chain of the multispecific antibody, the polynucleotide encoding a light chain of the multispecific antibody, or both thereof. Another embodiment provides a recombinant (host) cell transfected with the recombinant vector.

Another embodiment provides a method of preparing the multispecific antibody, comprising expressing the polynucleotide encoding a heavy chain of the multispecific antibody, the polynucleotide encoding a light chain of the multispecific antibody in a cell. The step of expressing the polynucleotide may be conducted by culturing the cell comprising the polynucleotide (for example, in a recombinant vector) under a condition allowing the expression of the polynucleotide. The method may further comprise isolating and/or purifying the multispecific antibody from the cell culture, after the step of expressing or culturing.

### EXAMPLES

Hereafter, the present invention will be described in detail by examples.

The following examples are intended merely to illustrate the invention and are not construed to restrict the invention.

### Example 1: Preparation of Anti-PD-L1 Monoclonal Antibodies

1.1. Preparation of Anti-Human-PD-L1 Mouse Monoclonal Antibodies and Analysis Thereof

Anti-human-PD-L1 mouse monoclonal antibodies were generated using the hybridoma technology, as disclosed in International Application Publication WO2017-215590.

The amino acid and polynucleotide sequences of the variable regions of the hybridoma supernatants, named Hybridoma HL1210-3, are provided in Table below.

TABLE 5

| | | SEQ ID |
|---|---|---|
| Name | Amino Acid Sequence | NO: |
| HL1210-3 | EVKLVESGGDLVKPGGSLKLSCAASGFTFSSYDMSWVRQTPEKSLEWVATIS | 63 |
| VH | DGGGYIYYSDSVKGRFTISRDNAKNNLYLQMSSLRSEDTALYICAREFGKRYA | |
| | LDYWGQGTSVTVSS | |
| HL1210-3 | DIVMTQSHKFMSTSVGDRVSISCKASQDVTPAVAWYQQKPGQSPKLLIYSTSS | 64 |
| VL | RYTGVPDRFTGSGSGTDFTFTISSVQAEDLAVYYCQQHYTTPLTFGAGTKLEL | |
| | K | |
| | | SEQ ID |
| Name | Nucleic Acid Sequence | NO: |
| HL1210-3 | GAAGTGAAACTGGTGGAGTCTGGGGGAGACTTAGTGAAGCCTGGAGGGT | 65 |
| VH | CCCTGAAACTCTCCTGTGCAGCCTCTGGATTCACTTTCAGTAGCTATGACA | |
| | TGTCTTGGGTTCGCCAGACTCCGGAGAAGAGTCTGGAGTGGGTCGCAACC | |
| | ATTAGTGATGGTGGTGGTTACATCTACTATTCAGACAGTGTGAAGGGGCGA | |
| | TTTACCATCTCCAGAGACAATGCCAAGAACAACCTGTACCTGCAAATGAG | |
| | CAGTCTGAGGTCTGAGGACACGGCCTTGTATATTTGTGCAAGAGAATTTGG | |
| | TAAGCGCTATGCTTTGGACTACTGGGGTCAAGGAACCTCAGTCACCGTCTC | |
| | CTCA | |
| HL1210-3 | GACATTGTGATGACCCAGTCTCACAAATTCATGTCCACATCGGTAGGAGAC | 66 |
| VL | AGGGTCAGCATCTCCTGCAAGGCCAGTCAGGATGTGACTCCTGCTGTCGC | |
| | CTGGTATCAACAGAAGCCAGGACAATCTCCTAAACTACTGATTTACTCCAC | |
| | ATCCTCCCGGTACACTGGAGTCCCTGATCGCTTCACTGGCAGTGGATCTGG | |
| | GACGGATTTCACTTTCACCATCAGCAGTGTGCAGGCTGAAGACCTGGCAG | |
| | TTTATTACTGTCAGCAACATTATACTACTCCGCTCACGTTCGGTGCTGGGAC | |
| | CAAGCTGGAGCTGAAA | |

1.2. Humanization of the HL1210-3 Mouse mAb

The mAb HL1210-3 variable region genes were employed to create a humanized Mab, per the methods commonly employed in the art and as disclosed in International Application Publication WO 2017-215590.

The amino acid and nucleotide sequences of some of the resultant humanized antibody are shown in SEQ ID NO. 67 to SEQ ID NO. 120.

The humanized VH and VK (VL kappa) genes were produced synthetically and then respectively cloned into vectors containing the human gamma 1 and human kappa constant domains. The pairing of the human VH and the human VK created the 40 humanized antibodies (see Table 6 to 9).

TABLE 6

| | VH | | | | | | |
|---|---|---|---|---|---|---|---|
| | Hu1210 | Hu1210 | Hu1210 | Hu1210 | Hu1210 | Hu1210 | Hu1210 |
| VK | VH.1 | VH.1a | VH.1b | VH.2 | VH.2a | VH2.b | VH |

| | | | | | | |
|---|---|---|---|---|---|---|
| Hu1210 | Hu1210-1 | Hu1210-2 | Hu1210-3 | Hu1210-4 | Hu1210-5 | |
| VK.1 | | | | | | |
| Hu1210 | Hu1210-7 | Hu1210-8 | Hu1210-9 | Hu1210-10 | Hu1210-11 | |
| VK.1a | | | | | | |
| Hu1210 | | | | | | H1210 |
| VK | | | | | | chimera |

TABLE 7

| | VH | | | | |
|---|---|---|---|---|---|
| | Hu1210 | Hu1210 | Hu1210 | Hu1210 | Hu1210 |
| VK | VH.3 | VH.3a | VH.4 | VH.4a | VH.4b |
| Hu1210 | Hu1210-13 | Hu1210-14 | Hu1210-15 | Hu1210-16 | Hu1210-17 |
| VK.1 | | | | | |
| Hu1210 | Hu1210-18 | Hu1210-19 | Hu1210-20 | Hu1210-21 | Hu1210-22 |
| VK.1a | | | | | |

TABLE 8

| | VH | | | | |
|---|---|---|---|---|---|
| | | HU1210 | HU1210 | HU1210 | HU1210 |
| VK | | VH.5a | VH.5b | VH.5c | VH.5d |
| Hu1210 | Hu1210-23 | Hu1210-27 | Hu1210-31 | Hu1210-32 | Hu1210-36 |
| VK.2 | | | | | |
| Hu1210 | Hu1210-24 | Hu1210-28 | | Hu1210-33 | Hu1210-37 |
| VK.2a | | | | | |
| Hu1210 | Hu1210-25 | Hu1210-29 | | Hu1210-34 | Hu1210-38 |
| VK.2b | | | | | |
| Hu1210 | Hu1210-26 | Hu1210-30 | | Hu1210-35 | Hu1210-39 |
| VK.2c | | | | | |

1.3. Preparation of Full Human Anti-PD-L1 Antibody

TABLE 9

VH

| VK | Hu1210 VH.4c | Hu1210 VH.4d | Hu1210 VH.4e |
|---|---|---|---|
| Hu1210 | Hu1210-40 | Hu1210-41 | Hu1210-42 |
| VK.1 | | | |

Full human anti-PD-L1 antibody also has been screened from a phage library.

Antigen: human PD-L1 extracellular domain (ECD) avi-His-biotion labeled protein (B3568B, Biointron).

Preparation of full human naïve phage library: The phage library was constructed by using phagemid vectors which consisted of antibody gene fragments that were amplified from PBMCs of healthy human subjects. It was constructed as a Fab phage library. The library size was 2×10¹¹.

Phage library solution panning against PD-L1 ECD protein. The phage libraries first underwent negative screening by incubating with BSA-coated streptavidin Dynabeads. The resulting phages were incubated with PD-L1-ECD-avi-his-biotin protein and washed by Kingfihser magnetic beads system. The binders were eluted by trypsin. The eluted phages (output 1) were subsequently tested for their titer to bind antigen and co-cultured with E. coli. There were three rounds of panning and screening. The titers of output 2 and output 3 were significantly increased.

Single clones were cherry picked from output 2 and 3 and then cultured in 96 deep well plate. The culture supernatant was subject to IgG concentration and antigen binding titer evaluation. 277 positive clones were selected and subject to sequencing. Post sequence analysis 128 unique sequences were identified. All these clones were subjected to ELISA binding analysis. 17 top sequences were identified, and B12 clone has been selected. Six (6) CDR sequences of B12 are as shown in Table 10, and sequences of heavy and light variable regions of B12 are as shown in Table 11.

TABLE 10

| B12 | Sequence | SEQ ID NO: |
|---|---|---|
| VH CDR1 | SYWMS | 294 |
| VH CDR2 | NIKQDGSEKYYVDSVKG | 295 |
| VH CDR3 | VALWDDAFDI | 296 |
| VL CDR1 | RASRGISSWLA | 297 |
| VL CDR2 | KASSLES | 298 |
| VL CDR3 | QQSSSIPLT | 299 |

TABLE 11

| | | SEQ |
|---|---|---|
| | | ID |
| B12 | Sequence | NO: |
| Heavy | QVQLLESGGGLVQPGGSLRLSCAASGFTFSSYWMSWV | 211 |
| Chain | RQAPGKGLEWVANIKQDGSEKYYVDSVKGRFTISRDN | |
| Variable | AKNSLYLQMNSLRAEDTAVYYCARVALWDDAFDIWGQ | |
| Region | GTMVTVSS | |
| (VH) | | |
| Light | DIQMTQSPSTLSASVGDRVIITCRASRGISSWLAWYQ | 209 |
| Chain | QKPGKAPNLLISKASSLESGVPSRFSGSGSGTDFTLT | |
| Variable | ISSLQPEDFATYYCQQSSSIPLTFGGGTKVEIK | |
| Region | | |
| (VL) | | |

1.4. Identification of PD-L1 Epitope

This study was conducted to identify amino acid residues involved in the binding of PD-L1 to the antibodies of the present disclosure.

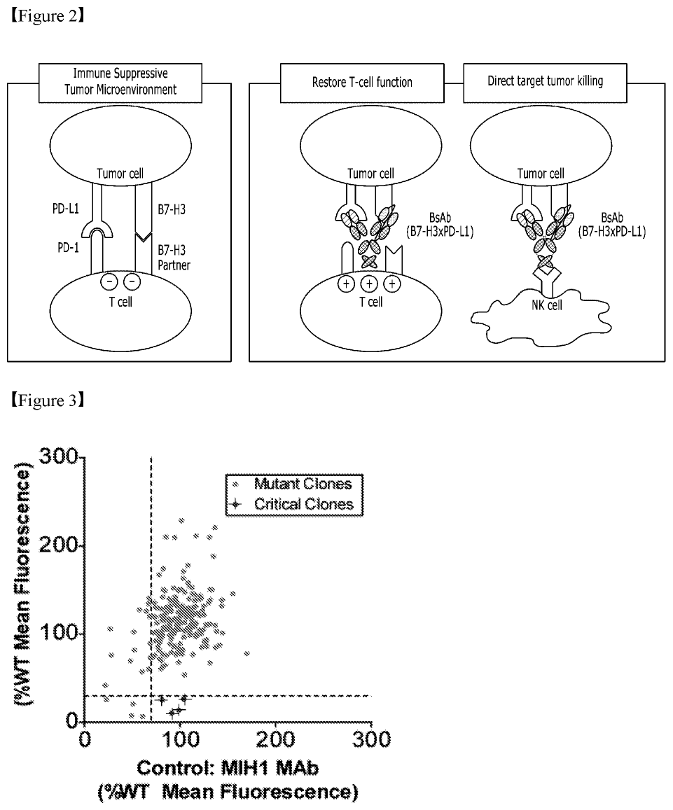

An alanine-scan library of PD-L1 was constructed. Briefly, 217 mutant clones of PD-L1 were generated on Integral Molecular's protein engineering platform. Binding of Hu1210-41 Fab to each variant in the PD-L1 mutation library was determined, in duplicate, by high-throughput flow cytometry. Each raw data point had background fluorescence subtracted and was normalized to reactivity with PD-L1 wild-type (WT). For each PD-L1 variant, the mean binding value was plotted as a function of expression. To identify preliminary critical clones (circles with crosses), thresholds (dashed lines) of >70% WT binding to control MAb (MIH1 Mab, in house prepared) and <30% WT reactivity to Hu1210-41 Fab were applied (FIG. 3). Y134, K162, and N183 of PD-L1 were identified as required residues for Hu1210-41 binding. The low reactivity of N183A clone with Hu1210-41 Fab suggests that it is the major energetic contributor to Hu1210-41 binding, with lesser contributions by Y134 and K162.

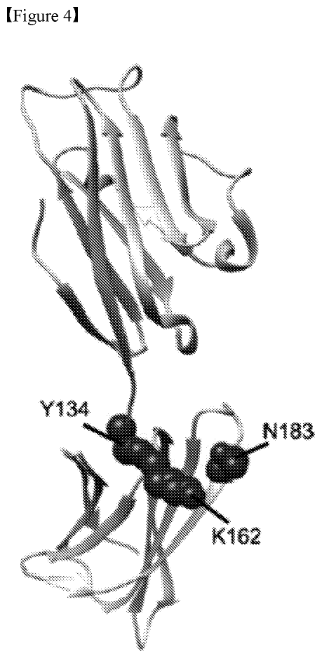

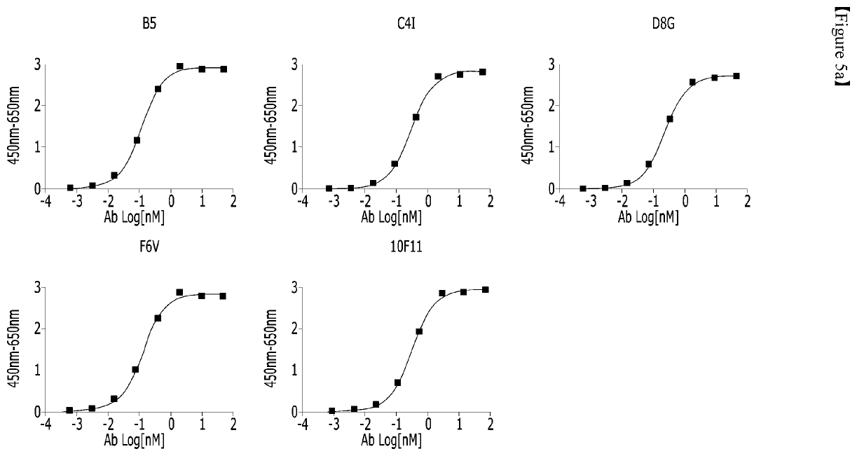

The critical residues (spheres) were identified on a 3D PD-L1 structure, as illustrated in FIG. 4. These residues, Y134, K162, and N183, therefore, constitute epitopes of PD-L1 responsible for binding to antibodies of various embodiments of the present disclosure.

It is interesting to note that Y134, K162, and N183 are all located within the IgC domain of the PD-L1 protein. Both PD-1 and PD-L1's extracellular portions have an IgV domain and an IgC domain. It is commonly known that PD-L1 binds to PD-1 through bindings between their IgV domains. Unlike such conventional antibodies, however, Hu1210-41 binds to the IgC domain, which would have been expected to be ineffective in inhibiting PD-1/PD-L1 binding. This different epitope of Hu1210-41, surprisingly, likely contributes to the excellent activities of Hu1210-41.

1.5. Antibody Engineering of Anti-PD-L1 Antibody

Examples 1.5 attempted to identify further improved antibodies based on Hu1210-41 using mutagenesis.

Four sub-libraries were constructed for antibody engineering of anti-PD-L1 monoclonal antibody, using either of the following strategies. In strategy 1, mutagenesis of heavy chain variable domain VH CDR3 or VL-CDR3 was perform by highly random mutation. In strategy 2, two CDR combination libraries composed of (VH-CDR3, VL-CDR3 and VL-CDR1) or (VH-CDR1, VH-CDR2 and VL-CDR2) were generated by CDR walking with controlled mutation rates.

Bio-Panning: the phage panning methods were adapted by shortening the incubation/binding time prior to the harsh washing condition. Briefly, 100 μl magnetic streptavidin beads (Invitrogen, USA) were blocked with 1 ml of MPBS for 1 hr at room temperature. In another tube, library phage was pre-incubated (5×10\hat{ }11˜12 for each round) with 100 μl magnetic streptavidin beads in 1 ml of MPBS to remove unwanted binders. Magnet particle concentrator was used to separate the phage and beads. The biotinylated PD-L1 protein was added to the phage and incubated 2 h at room temperature, and gently mixed using an over-head shaker. Beads carrying phage from the solution were separated in the magnetic particle concentrator and the supernatant was discarded. The beads were washed with fresh wash buffer, ten times with PBST and ten times with PBS (pH7.4). 0.8 ml, 0.25% Trypsin in PBS (Sigma, USA) was added and incubated for 20 min at 37° C. to elute the phage. The output phage was titrated and rescued for next round panning, decreasing antigen concentration round by round.

ELISA Screening and on/Off Rate Ranking

Clones were picked and induced from the desired panning output; phage ELISA was conducted for primary screening; positive clones were analyzed by sequencing; unique hotspots were found.

The Table below shows the mutations identified. As shown below, the hotpot mutation residues and/or their substitutes are underlined.

TABLE 12

| | CDR-H1 (SEQ ID No.) | CDR-H2 (SEQ ID No.) | CDR-H3 (SEQ ID No.) |
|---|---|---|---|
| WT | SYDMS (1) | TISDAGGYIYYSDSVKG (2) | EFGKRYALDY (4) |
| (H12) | | | |
| B3 | SYDMS (1) | TISDAGGYIYYRDSVKG (3) | EFGKRYALDY (4) |
| C4 | SYDMS (1) | TISDAGGYIYYRDSVKG (3) | EFGKRYALDS (6) |
| B1 | SYDMS (1) | TISDAGGYIYYRDSVKG (3) | EIFNRYALDY (7) |
| B6 | SYDMS (1) | TISDAGGYIYYRDSVKG (3) | ELPWRYALDY (5) |
| C3 | SYDMS (1) | TISDAGGYIYYRDSVKG (3) | ELHFRYALDY (8) |
| C6 | SYDMS (1) | TISDAGGYIYYRDSVKG (3) | ELYFRYALDY (9) |
| A1 | SYDMS (1) | TISDAGGYIYYRDSVKG (3) | ELLHRYALDY (10) |
| A2 | SYDMS (1) | TISDAGGYIYYRDSVKG (3) | ELRGRYALDY(ll) |
| A3 | SYDMS (1) | TISDAGGYIYYRDSVKG (3) | EFGKRYALDY (4) |
| | CDR-L1 (SEQ ID No.) | CDR-L2 (SEQ ID No.) | CDR-L3 (SEQ ID No.) |
| WT | KASQDVTPAVA (12) | STSSRYT (15) | QQHYTTPLT (16) |
| B3 | KAKQDVTPAVA (13) | STSSRYT (15) | MQHYTTPLT (17) |
| C4 | KASQDVWPAVA (14) | STSSRYT (15) | QQHSTTPLT (18) |
| B1 | KASQDVTPAVA (12) | STSSRYT (15) | QQHYTTPLT (16) |
| B6 | KASQDVTPAVA (12) | STSSRYT (15) | QQHYTTPLT (16) |
| C3 | KASQDVTPAVA (12) | STSSRYT (15) | QQHYTTPLT (16) |
| C6 | KASQDVTPAVA (12) | STSSRYT (15) | QQHYTTPLT (16) |
| A1 | KASQDVTPAVA (12) | STSSRYT (15) | QQHYTTPLT (16) |
| A2 | KASQDVTPAVA (12) | STSSRYT (15) | QQHYTTPLT (16) |
| A3 | KASQDVTPAVA (12) | STSSRYT (15) | QQHSDAPLT (19) |

The amino acid sequences of the variable regions of these antibodies are shown in Tables below.

TABLE 13

| | | SEQ ID |
|---|---|---|
| Name | Amino Acid Sequence | NO: |
| WT-VH | EVQLVESGGGLVQPGGSLRLSCAASGFTFSSYDMSWVRQAPGKSLEWVATISD | 121 |
| | AGGYIYYSDSVKGRFTISRDNAKNSLYLQMNSLRDEDTAVYICAREFGKRYAL | |
| | DYWGQGTTVTVSS | |
| WT-VK | DIQMTQSPSSLSASVGDRVTITCKASQDVTPAVAWYQQKPGKAPKLLIYSTSSR | 122 |
| | YTGVPSRFSGSGSGTDFTFTISSLQPEDIATYYCQQHYTTPLTFGQGTKLEIK | |
| B3-VH | EVQLVESGGGLVQPGGSLRLSCAASGFTFSSYDMSWVRQAPGKSLEWVATISD | 123 |
| | AGGYIYYRDSVKGRFTISRDNAKNSLYLQMNSLRDEDTAVYICAREFGKRYAL | |
| | DYWGQGTTVTVSS | |
| B3-VK | DIQMTQSPSSLSASVGDRVTITCKAKQDVTPAVAWYQQKPGKAPKLLIYSTSSR | 124 |
| | YTGVPSRFSGSGSGTDFTFTISSLQPEDIATYYCMQHYTTPLTFGQGTKLEIK | |
| C4-VH | EVQLVESGGGLVQPGGSLRLSCAASGFTFSSYDMSWVRQAPGKSLEWVATISD | 125 |
| | AGGYIYYRDSVKGRFTISRDNAKNSLYLQMNSLRDEDTAVYICAREFGKRYAL | |
| | DSWGQGTTVTVSS | |
| C4-VK | DIQMTQSPSSLSASVGDRVTITCKASQDVWPAVAWYQQKPGKAPKLLIYSTSSR | 126 |
| | YTGVPSRFSGSGSGTDFTFTISSLQPEDIATYYCQQHSTTPLTFGQGTKLEIK | |
| B1-VH | EVQLVESGGGLVQPGGSLRLSCAASGFTFSSYDMSWVRQAPGKSLEWVATISD | 127 |
| | AGGYIYYRDSVKGRFTISRDNAKNSLYLQMNSLRDEDTAVYICAREIFNRYAL | |
| | DYWGQGTTVTVSS | |
| B1-VK | DIQMTQSPSSLSASVGDRVTITCKASQDVTPAVAWYQQKPGKAPKLLIYSTSSR | 128 |
| | YTGVPSRFSGSGSGTDFTFTISSLQPEDIATYYCQQHYTTPLTFGQGTKLEIK | |
| B6-VH | EVQLVESGGGLVQPGGSLRLSCAASGFTFSSYDMSWVRQAPGKSLEWVATISD | 129 |
| | AGGYIYYRDSVKGRFTISRDNAKNSLYLQMNSLRDEDTAVYICARELPWRYA | |
| | LDYWGQGTTVTVSS | |
| B6-VK | DIQMTQSPSSLSASVGDRVTITCKASQDVTPAVAWYQQKPGKAPKLLIYSTSSR | 130 |
| | YTGVPSRFSGSGSGTDFTFTISSLQPEDIATYYCQQHYTTPLTFGQGTKLEIK | |
| C3-VH | EVQLVESGGGLVQPGGSLRLSCAASGFTFSSYDMSWVRQAPGKSLEWVATISD | 131 |
| | AGGYIYYRDSVKGRFTISRDNAKNSLYLQMNSLRDEDTAVYICARELHFRYAL | |
| | DYWGQGTTVTVSS | |
| C3-VK | DIQMTQSPSSLSASVGDRVTITCKASQDVTPAVAWYQQKPGKAPKLLIYSTSSR | 132 |
| | YTGVPSRFSGSGSGTDFTFTISSLQPEDIATYYCQQHYTTPLTFGQGTKLEIK | |
| C6-VH | EVQLVESGGGLVQPGGSLRLSCAASGFTFSSYDMSWVRQAPGKSLEWVATISD | 133 |
| | AGGYIYYRDSVKGRFTISRDNAKNSLYLQMNSLRDEDTAVYICARELYFRYAL | |
| | DYWGQGTTVTVSS | |
| C6-VK | DIQMTQSPSSLSASVGDRVTITCKASQDVTPAVAWYQQKPGKAPKLLIYSTSSR | 134 |
| | YTGVPSRFSGSGSGTDFTFTISSLQPEDIATYYCQQHYTTPLTFGQGTKLEIK | |
| A1-VH | EVQLVESGGGLVQPGGSLRLSCAASGFTFSSYDMSWVRQAPGKSLEWVATISD | 135 |
| | AGGYIYYRDSVKGRFTISRDNAKNSLYLQMNSLRDEDTAVYICARELLHRYAL | |
| | DYWGQGTTVTVSS | |
| A1-VK | DIQMTQSPSSLSASVGDRVTITCKASQDVTPAVAWYQQKPGKAPKLLIYSTSSR | 136 |
| | YTGVPSRFSGSGSGTDFTFTISSLQPEDIATYYCQQHYTTPLTFGQGTKLEIK | |
| A2-VH | EVQLVESGGGLVQPGGSLRLSCAASGFTFSSYDMSWVRQAPGKSLEWVATISD | 137 |
| | AGGYIYYRDSVKGRFTISRDNAKNSLYLQMNSLRDEDTAVYICARELRGRYAL | |
| | DYWGQGTTVTVSS | |
| A2-VK | DIQMTQSPSSLSASVGDRVTITCKASQDVTPAVAWYQQKPGKAPKLLIYSTSSR | 138 |
| | YTGVPSRFSGSGSGTDFTFTISSLQPEDIATYYCQQHYTTPLTFGQGTKLEIK | |
| A3-VH | EVQLVESGGGLVQPGGSLRLSCAASGFTFSSYDMSWVRQAPGKSLEWVATISD | 139 |
| | AGGYIYYRDSVKGRFTISRDNAKNSLYLQMNSLRDEDTAVYICAREFGKRYAL | |
| | DYWGQGTTVTVSS | |
| A3-VK | DIQMTQSPSSLSASVGDRVTITCKASQDVTPAVAWYQQKPGKAPKLLIYSTSSR | 140 |
| | YTGVPSRFSGSGSGTDFTFTISSLQPEDIATYYCQQHSDAPLTFGQGTKLEIK | |

TABLE 14

| Anti- | | SEQ |
|---|---|---|
| body | | ID |
| No. | Amino Acid Sequence of VH | NO: |
| H12 | EVQLVESGGGLVQPGGSLRLSCAASGFTFSSYDMSWVR | 141 |
| | QAPGKSLEWVATISDAGGYIYYSDSVKGRFTISRDNAK | |
| | NSLYLQMNSLRDEDTAVYICAREFGKRYALDYWGQGTT | |
| | VTVSS | |
| B6 | EVQLVESGGGLVQPGGSLRLSCAASGFTFSSYDMSWVR | 142 |
| | QAPGKSLEWVATISDAGGYIYYRDSVKGRFTISRDNAK | |
| | NSLYLQMNSLRDEDTAVYICARELPWRYALDYWGQGTT | |
| | VTVSS | |

TABLE 15

| Anti- | | SEQ |
|---|---|---|
| body | | ID |
| No. | Amino Acid Sequence of VL | NO: |
| H12 | DIQMTQSPSSLSASVGDRVTITCKASQDVTPAVAWYQQK | 143 |
| | PGKAPKLLIYSTSSRYTGVPSRFSGSGSGTDFTFTISSL | |
| | QPEDIATYYCQQHYTTPLTFGQGTKLEIK | |
| B6 | DIQMTQSPSSLSASVGDRVTITCKASQDVTPAVAWYQQK | 144 |
| | PGKAPKLLIYSTSSRYTGVPSRFSGSGSGTDFTFTISSL | |
| | QPEDIATYYCQQHYTTPLTFGQGTKLEIK | |

1.6. Protein Kinetic for PD-L1

To explore the binding kinetics of the humanized antibody, this example performed the affinity ranking by using Biacore. As shown in Table below, H12 and B6.

TABLE 16

| Antibody | KD (M) | kon(1/Ms) | kdis(1/s) | Chi |
|---|---|---|---|---|
| H12 | 6.122E−09 | 7.124E+04 | 4.361E−04 | 0.0415 |
| B6 | 4.248E−09 | 9.827E+04 | 4.175E−04 | 0.0766 |

As shown in Table above, the anti-PD-L1 antibodies tested show high PD-L1 binding affinities.

### Example 2. Preparation of Anti-B7-H3 Monoclonal Antibodies

2.1. Preparation of Anti-B7-H3 Monoclonal Antibodies and Analysis Thereof

2.1.1. Preparation of Antigen

An antigen used for phage display performance for preparation of anti-B7-H3 antibody was purchased and used. In case of human B7-H3, the 1st to 461th of the amino acid sequence of NP_001019907.1 are comprised and a recombinant B7-H3 protein in which a histidine-tag (His tag) is linked to the C terminal (2318-B3/CF, R&D Systems) was used.

An antigen used for ELISA analysis, SPR analysis or T cell activity analysis of the following examples was purchased and used as follows. In case of human B7-H3, the 1st to 461^{th} of the amino acid sequence of NP_001019907.1 are comprised and a recombinant B7-H3 protein in which a histidine-tag (His tag) is linked to the C terminal and a protein in which Fc region of human IgG1 is linked to the C terminal (Sino Biological, 11188-H02H) were used.

2.1.2: Antibody Sorting Preparation Through Phage Library Screening

Preparation of Library Phage

After culturing 2×10¹⁰ E. coli having a human-derived scFv (single-chain variable fragment) library (Mol. Cells OT, 225-235, Feb. 28, 2009) gene having the binding variety to various antigens in a medium comprising 2×YT (Amresco, J902-500G), ampicillin 100 g/ml, and 2% glucose (sigma, G7021) at 37° C. for 2 hours to 3 hours so that OD600 value is 0.5 to 0.7. After infecting a helper phage by the cultured E. coli, it was cultured in a 2×YT [2×YT, ampicilin 100 μg/ml, 1 mM IPTG (Duchefa, I1401)] medium at 30° C. for 16 hours and thereby phage packaging was induced. Then, after centrifuging the cultured cells under the condition of 4° C., 4500 rpm for 20 minutes, 4% PEG 8000 (sigma, P2139) and 3% NaCl (Samchun, S2097) were added to a supernatant and melted well, and then it was reacted on ice for 1 hour. After centrifuging under the condition of 4° C., 8000 rpm again, PBS (Phosphate buffered saline, Gibco 10010-023) was added to a pellet and it was suspended. After the suspension was centrifuged under the condition of 4° C., 1200 rpm for 10 minutes, the supernatant was put into a new tube and it was stored at 4° C. before use.

Panning Through Phage Display

To sort antibodies binding to a human B7-H3 protein, using the recombinant B7-H3 protein, with which the histidine-tag (His tag) is linked, of Example 2, panning was progressed 3 times in total as follows.

Specifically, a protein was absorbed on a surface of test tube under conditions of 37° C., 200 rpm for 1 hr, by adding 2 μg/ml concentration of recombinant human B7-H3 protein of 1 ml into an immunotube (maxisorp 444202). Then, a supernatant was removed and a solution comprising 3% skim milk was added to the test tube and it was reacted at a room temperature for 1 hr. Though this, skim milk was adsorbed on the surface of the immunotube to which the recombinant human B7-H3 protein was not adsorbed, thereby blocking non-specific binding. After removing the supernatant, 1012 CFU of phage library prepared in Example 2.1.2 was mixed in the solution comprising 3% skim milk and put into the immunotest, and it was reacted under the conditions of 37° C., 150 rpm for 1 hr, so that the phages specific to human B7-H3 protein bound to an antigen.

Then, non-specifically bound phages were washed with PBS-T (Phosphate buffered saline-0.05% Tween 20) solution and removed, and the remained antigen-specific phage antibodies were collected by adding 1 ml of 100 mM triethylamine solution. After neutralizing the collected phages with 1M Tris buffer solution (pH 7.4) as the pH of triethylamine solution was low, it was infected to ER2537 E. coli grown as 0.8˜1 at OD600 under the conditions of 37° C., 120 rpm for 1 hour and 30 minutes. The culture solution was centrifuged under the conditions of 4° C., 4500 rpm for 15 min and the supernatant was removed, and sunk cells were cultured at 37° C. for 16 hr or more by smearing infected E. coli on a 2×YT agar medium comprising ampicillin. Next day, all the cultured E. coli was scraped out and suspended in 5 ml of 2×YT ampicillin culture solution, and 505 glycerol was added, and a part was stored at −80° C. and the other was used for preparing a phage for the next experiment. After inoculating 20 μl of cultured E. coli in a 2×TB comprising ampicillin and growing it, a helper phage was infected and panning were repeated twice more, thereby amplifying and concentrating a human B7-H3 protein-specific phage pool.

Single Clone Screening

To sort monoclonal antibodies specifically binding to human B7-H3 protein from the phage pool obtained through the panning, the experiment as follows was performed.

To isolate monoclones from the concentrated pool, after smearing the phage pool on a LB-ampicillin agar medium and culturing, a single colony was secured. Then, after inoculating monoclones on a 96-deep well plate in which 200 μl of super broth (SB) medium was put per well and cultivating overnight, a part was transferred into other plate to make cell stock. 1 mM IPTG was put into the remained cell culture solution and it was cultured at 30° C. for 16 hrs, to induce production of scFv. After the cultured culture solution was centrifuged under the conditions of 4° C., 6000 rpm, the supernatant was discarded and only cells were obtained, and then cells were lysated using TES solution and then centrifuged again, thereby obtaining only the supernatant to use.

Then, clones expressing a soluble monoclonal scFv which binds to B7-H3-His antigen (2318-B3/CF, R&D Systems) were selected by using the ELISA method as follows (Steinberger. Rader and Barbas III. 2000. Phage display vectors. In: Phage Display Laboratory Manual. 1sted. Cold Spring Harbor Laboratory Press. NY USA. pp. 11.9-11.12). Specifically, the recombinant human B7-H3-his protein prepared in Example 2 of 100 ng per well was put on a 96-well plate (Nunc-Immuno Plates, NUNC, Rochester, N.Y., USA) and it was adsorbed at 4° C. overnight. Next day, after washing the protein with PBST (Phosphate buffered saline-0.05% Tween 20), to prevent non-specific binding, PBS buffer solution comprising 3% BSA of 200 μL per well was put and it was reacted at 37° C. for 2 hours. Then, after washing it with PBST again, the supernatant comprising phages centrifuged and prepared in advance of 100 μl per well was put and it was reacted at 37° C. for about 1 hr. Then, after washing it with PBST, to detect phages bound to human B7-H3, the anti-HA HRP (Horseradish peroxidase)-binding antibody (Roche, 12 013 819 001) was diluted in PBS comprising 100 BSA by 1:5000, and 100 μl per well was put and it was reacted at 37° C. for about 1 hr. After washing it with PBST again, TMB (Tetramethylbenzidine, Thermo, 34028) 100 μl was put to develop color. After reacting at RT for 5˜10 min, 50 ml of 1N H₂SO₄ was put to finish the reaction. The absorbance at 450 nm was measured to sort clones of which value was 1.0 or more.

Therefrom, 7 antibody clones binding to the recombinant human B7-H3 protein (B5, C4I, D8G, F6V, 10F11, D8G M1 and D8G M3) were sorted, and the amino acid sequences and CDR sequences of heavy chain variable and light chain variable regions of each antibody were as the following tables.

TABLE 17

| | CDR Sequences of Heavy Chain Variable (VH) | | | | | | |
|---|---|---|---|---|---|---|---|
| | CDRH1 | | CDRH2 | | CDRH3 | | VH |
| | | SEQ | | SEQ | | SEQ | SEQ |
| | | ID | | ID | | ID | ID |
| Clone | Sequence | NO | Sequence | NO | Sequence | NO | NO |
| B5 | DYAMS | 20 | SISSGSGSIYYADSVKG | 24 | NLIPLDY | 30 | 51 |
| C4I | GYYMS | 21 | LISPSSGSIYYADSVKG | 25 | GLTKFDY | 31 | 52 |
| D8G | SYSMS | 22 | GIYSDGSNTYYADSVKG | 26 | MLHRFDY | 32 | 53 |
| F6V | DYAMS | 20 | GISPGGSNTYYADSVKG | 27 | DAWIARLLLFDY | 33 | 54 |
| 10F11 | SYGMS | 23 | GIYSGGSSKYYADSVKG | 28 | NRLRFDY | 34 | 55 |
| D8GM1 | SYSMS | 22 | GIYSDASNTYYADSVKG | 29 | MLHRFDY | 32 | 56 |
| D8GM3 | SYSMS | 22 | GIYSDASNTYYADSVKG | 29 | MLHRFDY | 32 | 56 |

TABLE 18

| | CDR Sequences of Light Chain Variable (VL) | | | | | | |
|---|---|---|---|---|---|---|---|
| | CDRL1 | | CDRL2 | | CDRL3 | | VL |
| | | SEQ | | SEQ | | SEQ | SEQ |
| | | ID | | ID | | ID | ID |
| Clone | Sequence | NO | Sequence | NO | Sequence | NO | NO |
| B5 | SGSSSNIGSNAVS | 35 | YNSHRPS | 40 | GSWDASLNAYV | 46 | 57 |
| C4I | TGSSSNIGSNDVS | 36 | ANSHRPS | 41 | GSWDDSLSGYV | 47 | 58 |
| D8G | SGSSSNIGSNSVT | 37 | ADSQRPS | 42 | GTWDSSLNAYV | 48 | 59 |
| F6V | SGSSSNIGSNAVT | 38 | YNNKRPS | 43 | GTWDDSLSGYV | 49 | 60 |
| 10F11 | TGSSSNIGSNSVT | 39 | SDSHRPS | 44 | GTWDASLNAYV | 50 | 61 |
| D8GM1 | SGSSSNIGSNSVT | 37 | ADSQRPS | 42 | GTWDSSLNAYV | 48 | 59 |
| D8GM3 | SGSSSNIGSNSVT | 37 | ADVQRPS | 45 | GTWDSSLNAYV | 48 | 62 |

The nucleic acid sequences encoding the variable regions and CDR sequences were comprised in the following full-length nucleic acid sequences in the order of B5, C4I, D8G, F6V, 10F11, D8G M1, and D8G M3: SEQ ID NOs: 145 (heavy chain) and 151 (light chain); SEQ ID NOs: 146 (heavy chain) and 152 (light chain); SEQ ID NOs: 147 (heavy chain) and 153 (light chain); SEQ ID NOs: 148 (heavy chain) and 154 (light chain); SEQ ID NOs: 149 (heavy chain) and 155 (light chain); SEQ ID NOs: 150 (heavy chain) and 153 (light chain); and SEQ ID NOs: 150 (heavy chain) and 156 (light chain), respectively. In the nucleic acid sequences, the nucleic acid sequences encoding constant regions were SEQ ID NO: 158 to 160 (heavy chain), and SEQ ID NO: 161 to 163 (light chain).

### Example 2.2. Conversion of Anti-B7-H3 scFv into Full IgG Form and Production Thereof

2.2.1. Cloning of Anti-B7-H3 scFv into Full IgG Form

To convert each human B7-H3 specific monoclonal phage antibody, secured in Example 2.1, into a full IgG form, nucleic acids encoding heavy chain and light chain variable regions of each clone secured in Example 2.1 were synthesized (Genotech, Korea). Genes encoding human IgG1 subtype of heavy chain and light chain constant regions (SEQ ID NOs: 157 and 161, respectively) proteins (heavy chain constant regions SEQ ID NOs: 158 (C4I, D8G, 10F11, D8G M1, D8G M3 clone), 159 (B5 clone), 160 (F6V clone) and light chain constant regions 162 (C4I, D8G, 10F11, D8G M1, D8G M3 clone), 163 (B5 clone) and 164 (F6V clone)) were synthesized and were linked with the nucleic acid encoding each heavy chain and light chain variable region. The nucleic acids encoding light chain and heavy chain of each antibody were cloned in a pcDNA 3.1-based expression vector, respectively, and a vector encoding an antibody nucleic acid in a mammal cell line of CHO-S, etc. was secured. In addition, to use the conventional anti-B7-H3 antibody, Enoblituzumab as a comparison group antibody, the variable region sequence of the antibody was secured from the patent (U.S. Pat. No. 8,802,091) and the gene was secured, and it was cloned as same as the aforementioned method and named as 84D to use.

The IgG form of antibodies were disclosed as the following heavy chain and light chain full-length sequences in the order of B5, C4I, D8G, F6V, 10F11, D8G M1, and D8G M3: SEQ ID NOs: 145 (heavy chain) and 151 (light chain); SEQ ID NOs: 146 (heavy chain) and 152 (light chain); SEQ ID NOs: 147 (heavy chain) and 153 (light chain); SEQ ID NOs: 148 (heavy chain) and 154 (light chain); SEQ ID NOs: 149 (heavy chain) and 155 (light chain); SEQ ID NOs: 150 (heavy chain) and 153 (light chain); and SEQ ID NOs: 150 (heavy chain) and 156 (light chain), respectively.

2.2.2. Expression of Anti-B7-H3 Antibody

For expression of the anti-B7-H3 antibody, ExpiCHO-S™ (Thermo Fisher, A29127) cells developed by Theremo company were used, and the expression of the antibody was performed, following ExpiCHO™ Expression System Kit (Thermo Fisher, A29133) protocol of the manufacturer.

Briefly describing the preparation method, ExpiCHO-S cells were cultured under the condition of 120 rpm in a shaking incubator of 8% CO₂, 37° C. conditions. On the day of transfection, ExpiCHO-S cells were diluted by adding ExpiCHO™ Expression Medium (Thermo Fisher, A2910001) at a cell concentration of 6×10⁶ cells/ml and prepared.

Then, each vector expressing the heavy chain and light chain from Example 2.2.1 was diluted in OptiPRO™ SFM medium (Thermo Fisher, 12309050), in 1 μg per medium ml, respectively, and 3.2 μl per ml of ExpiFectamine™CHO included in ExpiCHO Expression system was diluted in OptiPRO™ SFM medium. The vector and ExpiFectamine™CHO mixture were mixed each other and reacted at a room temperature for 5 min, and then the mixture was put into the prepared cells and it was cultured under the conditions of 8% C02, 37° C., 120 rpm for 20 hrs. In 20 hrs, after adding 2.2 μl/ml and 240 μl/ml of Enhencer1, ExpiCHO™ Feed, both included in ExpiCHO™ Expression System Kit (Thermo Fisher, A29133), were added to cells, respectively, it was cultured under the conditions of 8% C02, 37° C., 120 rpm for about 7 days to 10 days.

After culturing, the cell culture solution was centrifuged under the conditions of 4° C., 6000 rpm for 30 min, and then the supernatant was isolated and refrigerated.

2.2.3. Separation and Purification of Anti-B7-H3 Antibody

After passing an equilibrium buffer solution (50 mM Tris-HCl, pH7.5, 100 mM NaCl) through Mab selectsure (GE healthcare, 5 ml) to equilibrate it, the culture solution of Example 2.2.2 through a column (Mab selectsure (GE healthcare, 5 ml)) in order to allow the expressed antibody to bind to the column. Then, after eluting it with a 50 mM Na-citrate (pH 3.4), 100 mM NaCl solution, it was neutralized by using 1 M Tris-HCl (pH 9.0) so that the final pH was 7.2. The buffer solution was exchanged with PBS (phosphate buffered saline, pH 7.4).

2.3. Analysis of Binding Specificity to B7-H3 of Anti-B7-H3 Antibody

2.3.1. Analysis of Binding Specificity to Recombinant B7-H3 Antigen of Anti-B7-H3 IgG Antibody (ELISA)

To confirm the specific binding capacity to B7-H3 antigen of anti-B7-H3 IgG antibodies selected and prepared in Examples 2.1 and 2.2, ELISA-based solution binding test was performed.

Specifically, after diluting the recombinant human B7-H3 protein at a concentration of 1 μg/ml and putting it into a 96-well plate (Nunc-Immuno Plates, NUNC) in 100 μl per well, it was reacted at 4° C. for 16 hrs for coating. The recombinant human B7-H3 protein used in Example 2.1 was used here.

Then, after removing the protein and washing it with PBST, a PBS buffer comprising 1% BSA (bovine serum albumin) was put at 200 μl per well and it was reacted at 37° C. for 2 hrs to block non-specific binding. Then, after diluting anti-B7-H3 antibodies prepared in Example 2.2 at a concentration of 10 μg/ml on the 96-well plate, 100 μl was put in each well and it was reacted at 37° C. for 1 hr. Then after it was washed with PBST. In order to detect antibodies bound to human B7-H3, HRP-connected anti-human IgG F(ab′)2 antibody (Goat anti-Human IgG F(ab′)2 Cross-Adsorbed Secondary Antibody, HRP, Pierce, 31414) was diluted by 1:10,000 in PBS comprising 1% bovine serum albumin (BSA), and 100 μl was put per well and it was reacted at 37° C. for about 1 hr. After washing it with PBST again, TMB (Tetramethylbenzidine, Sigma, T0440) 100 μl was put to develop color. After reacting it at RT for 5˜10 min, 50 μl of 1N H₂SO₄ was put to finish the reaction, and the absorbance at 450 nm and 650 nm was measured by using a micro plate reader (molecular device).

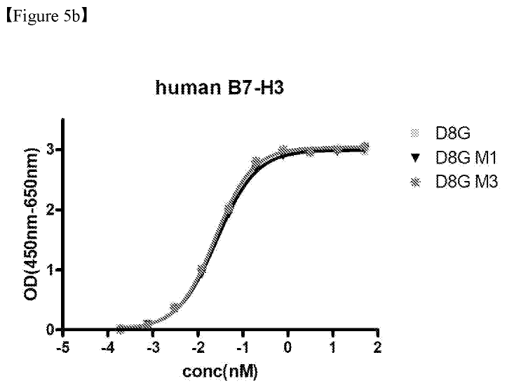

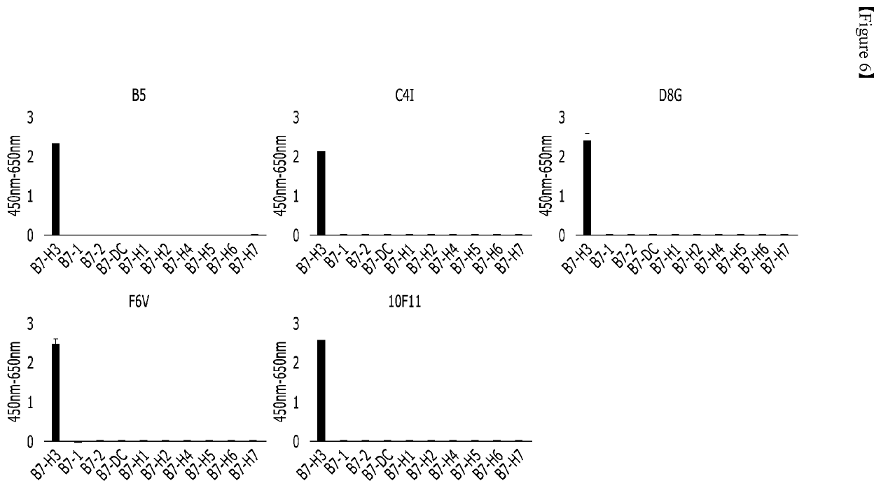

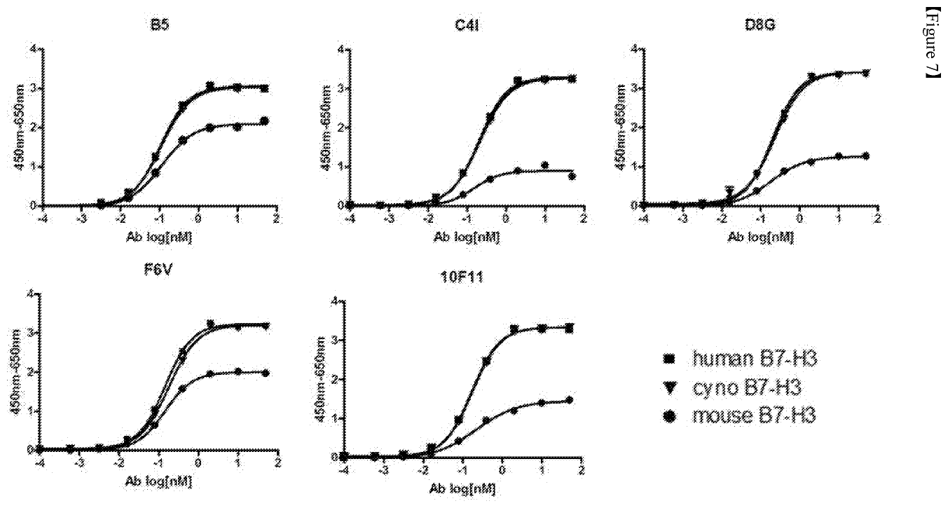

The result was described in FIGS. 5a and 5b. As the result of measuring the binding capacity using ELISA method, it was confirmed that anti-B7-H3 antibodies bound to an extracellular domain of human B7-H3 in a concentration-dependent manner.

2.3.2. Analysis of Binding Capacity to Other Proteins of B7 Family of Anti-B7-H3 Antibody

B7 family proteins share 20˜40% of identical amino acids each other, and have structural relevance such as repeatability of immunoglobulin domain. Thus, it was analyzed whether the anti-B7-H3 antibodies of the present invention specifically bind to B7-H3 protein, not to other B7 family proteins, as follows.

To confirm immune specific binding capacity, B7 family component proteins having structural similarity: B7-1(Sino Biological, Cat #: 10698-H08H), B7-2(Sino Biological, Cat #: 10699-H08H), B7-DC(Sino Biological, Cat #: 10292-H08H), B7-H1(Sino Biological, Cat #: 10084-H08H), B7-H2(Sino Biological, Cat #: 11559-H08H), B7-H4(Sino Biological, Cat #: 10738-H08H), B7-H5(Sino Biological, Cat #: 13482-H08H), B7-H6(Sino Biological, Cat #: 16140-H08H), B7-H7(Sino Biological, Cat #: 16139-H02H) were purchased and used.

Specifically, after diluting the recombinant human B7 family proteins at a concentration of 1 μg/ml and putting them in a 96-well plate (Nunc-Immuno Plates, NUNC) in 100 μl per well, it was reacted at 4° C. for 16 hrs for coating. The recombinant proteins used in Example 2.1 was used.

Then, after removing proteins and washing it with PBST, 200 μl of PBS buffer comprising 1% BSA (bovine serum albumin) was put per well and it was reacted at 37° C. for 2 hrs to block non-specific binding. Then, after diluting the anti-B7-H3 antibodies prepared in Example 2.2 in 10 μg/ml on a 96-well plate, 100 μl was put per well and it was reacted at 37° C. for 1 hr. Then after it was washed with PBST. In order to detect antibodies bound to an antigen, HRP-connected anti-human IgG F(ab′)₂ antibody (Goat anti-Human IgG F(ab′)₂ Cross-Adsorbed Secondary Antibody, HRP, Pierce, 31414) was diluted in PBS comprising 1% bovine serum albumin (BSA) by 1:10,000. 100 μl was put per well and it was reacted at 37° C. for about 1 hr. After washing it with PBST again, TMB (Tetramethylbenzidine, Sigma, T0440) 100 μl was put to develop color. After reacting it at RT for 5-10 min, 50 μl of H₂SO₄ was put to finish the reaction, and the absorbance at 450 nm and 650 nm was measured by using a micro plate reader (molecular device).

The result was described in FIG. 6. As the result of measuring the binding capacity using ELISA method, it was confirmed that the anti-B7-H3 antibody specifically bound to B7-H3 only, not to the other B7 family proteins.

2.3.3. Analysis of Cross-Species Reactivity to Human, Monkey and Mouse B7-H3 of Anti-B7-H3 Antibody

To estimate the antibody efficacy and immune regulator activity of the anti-B7-H3 antibody before progressing clinical to human, estimation in rodents or primates model is important. The sequence of human B7-H3 shares 90% or more identity in monkey and mouse. The cross-reactivity to mouse or monkey B7-H3 of the anti-B7-H3 antibodies of the present invention prepared in Example 2.2 was analyzed by the ELISA analysis method as follows.

To confirm the cross-species reactivity, antigens of a recombinant mouse B7-H3 protein in which a histidine tag (His tag) was linked to the C terminal (Sino Biological, Cat #: 50973-M08H) and a recombinant monkey B7-H3 protein in which Fc region of human IgG1 was linked to the C terminal (Sino Biological, Cat #: 90806-C02H) were purchased and used.

After diluting the recombinant human B7-H3, mouse B7-H3 and monkey B7-H3 proteins in a concentration of 1 μg/ml and putting them in a 96-well plate (Nunc-Immuno Plates, NUNC) as 100 μl per well, it was reacted at 4° C. for 16 hrs and coated. For the used recombinant proteins, the product purchased for analysis in Example 2.1 was used.

Then, after removing proteins and washing it with PBST, 200 μl of PBS buffer comprising 1% BSA (bovine serum albumin) per well was put and it was reacted at 37° C. for 2 hrs to block non-specific binding. Then, after diluting the anti-B7-H3 antibodies prepared in Example 2.2 at certain concentrations ranging from 10 μg/ml on a 96-well plate, 100 μl was put per well and it was reacted at 37° C. for 1 hr. Then, after washing it with PBST, to detect antibodies bound to human B7-H3, mouse B7-H3 and monkey B7-H3, HRP-connected anti-human IgG F(ab′)2 antibody (Goat anti-Human IgG F(ab′)2 Cross-Adsorbed Secondary Antibody, HRP, Pierce, 31414) was diluted in PBS comprising 1% bovine serum albumin (BSA) by 1:10,000, and 100 μl was put per well and it was reacted at 37° C. for about 1 hr. After washing it with PBST again, TMB (Tetramethylbenzidine, Sigma, T0440) 100 μl was put to develop color. After reacting it at RT for 5˜10 min, 50 μl of H₂SO₄ was put to finish the reaction, and the absorbance at 450 nm and 650 nm was measured by using a micro plate reader (molecular device).

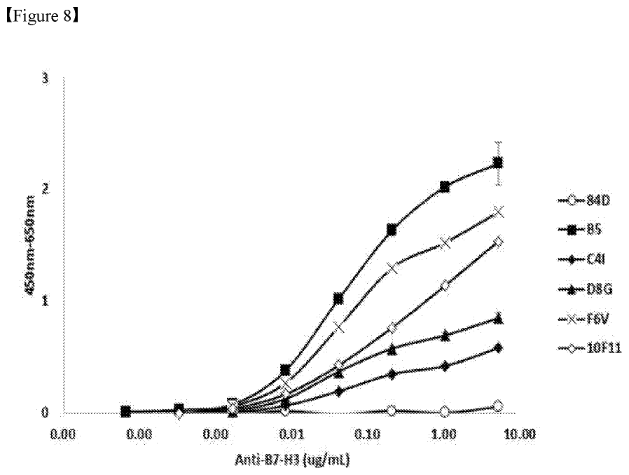

The result was described in FIG. 7 and FIG. 8. As the result of measuring the binding capacity using the ELISA method, it was confirmed that the anti-B7-H3 antibody specifically bound to human, monkey and mouse B7-H3s. The binding degrees of the anti-B7-H3 antibodies of the present invention to human and monkey B7-H3s were shown to be similar, but the binding degrees to mouse B7-H3 was relatively low (FIG. 7). It was observed that the binding degrees of the anti-B7-H3 antibodies to mouse B7-H3 were varying among the clones, and 84D antibody used as the comparison antibody did not bind to mouse B7-H3 protein (FIG. 8).

2.3.4. Measurement of Binding Capacity to Cell Surface Expression B7-H3 Antigen of Anti-B7-H3 Antibody

Then, through FACS analysis, the ability of the anti-B7-H3 antibody of the present invention prepared in Example 2.2 to bind to human B7-H3 expressed on a cell surface was measured.

For the experiment, the cancer cell lines expressing human B7-H3, MCF-7 (Human breast adenocarcinoma cell line, ATCC® HTB-22™), DLD1 (colorectal adenocarcinoma cell lines, ATCC® CCL-221™), HCC1954 (TNM stage IIA, grade 3, ductal carcinoma, ATCC® CRL-2338™), and HCT116 (colon cancer cell, ATCC® CCL-247™) and the cancer cell line, not expressing human B7-H3, Jurkat (acute T cell leukemia, ATCC® TIB-152™) were used.

Specifically, after dissociating each cell line and washing it with PBS buffer, the number of cells was counted and adjusted to 2×10⁵ cells per well, and prepared by putting 200 μl PBS. Each of anti-B7-H3 antibodies of Example 2.2 and comparison group antibody (84D) was reacted with the cells prepared in advance as diluted at a certain concentration of 10 μg/ml or more in PBS comprising 1% BSA, at 4° C. for 1 hr. After washing it twice using PBS buffer, the FITC-labeled anti-human Fc FITC (Goat anti-human IgG FITC conjugate, Fc specific, Sigma, F9512, concentration: 2.0 mg/ml) was diluted by 1:500 and treated in 100 μl per well, and it was reacted at 4° C. for 1 hr. The negative control group was treated with the FITC-labeled anti-human Fc FITC only. After washing it twice using PBS buffer again, the degree of binding of anti-B7-H3 IgG was measured using FACSCalibur device.

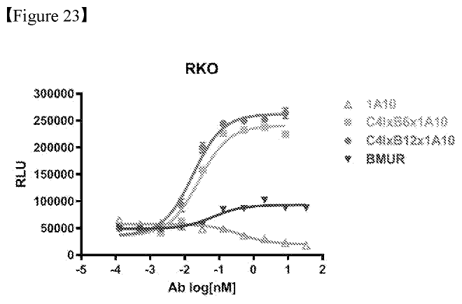

The result of the peak shift for the human B7-H3-monoclonal antibody-FITC binding in experimental groups in which each B7-H3 monoclonal antibody was treated was compared to the negative control group binding. The result was represented by the value for the peak shift in the experimental groups treated with B7-H3 monoclonal antibody as divided by the value for the peak shift in the negative control group (Mean Fluorescence Intensity Ratio), and described in FIG. 9 and FIG. 10. As the result of measuring the binding capacity using FACS method, it was confirmed that the anti-B7-H3 antibody specifically binds to human B7-H3 expressed on a cell surface in a concentration-dependent manner.

2.3.5. Measurement of Binding Capacity to Cell Surface Expression B7-H3 Antigen of Anti-B7-H3 IgG Antibody in Various Cancer Kinds

Then, through FACS analysis, whether the anti-B7-H3 antibody of the present invention binds to cell surface expression B7-H3 in various kinds of cancer cell lines was confirmed.

Using various kinds of cancer cells A2780 (human ovarian cancer, ECACC, 93112519), SKOV-3 (human ovarian adenocarcinoma, ATCC® HTB-77™), OVCAR-3 (human ovarian adenocarcinoma, ATCC® HTB-161™), HCT116 (colon cancer cell, ThermoFIshcer Sci), HT29 (olorectal adenocarcinoma, ATCC® HTB-38™), DLD-1 (colorectal adenocarcinoma cell lines, ATCC® CCL-221™), Calu-6 (Non-small-cell lung carcinoma, ATCC® HTB-56™), HCC1954 (TNM stage IIA, grade 3, ductal carcinoma, ATCC® CRL-2338™), HCC1187 (TNM stage IIA, ATCC® CLC-2322™), renal cancer cell line 786-0 (renal cell adenocarcinoma, ATCC® CRL-1932™), A498 (kidney carcinoma, ATCC® HTB-44™), Panc-1 (pancreas epithelioid carcinoma, TCC® CRL-1469™), NCI-N87 (gastric carcinoma, TCC® CRL-5822™), HeLa (cervix adenocarcinoma, ATCC® CCL-2™), JeKo-1 (Lymphoma, ATCC® CRL-3006™) and FACSCalibur (BD Biosciences) device, the degree of binding of anti-B7-H3 antibody to B7-H3 was measured as follows.

After dissociating each cell line and washing it with PBS buffer, the number of cells was counted and adjusted to 2×10⁵ cells/200 μl PBS, and then was treated with 10 μg/ml of B7-H3 monoclonal antibodies prepared in Example 2.2. Reaction was allowed at 4° C. for 1 hr. After washing the reacted cells in PBS, the FITC-labeled constant region (Fc)-specific antibody (Goat anti-human IgG FITC conjugate, Fc specific, Sigma, F9512, concentration: 2.0 mg/ml)) was diluted by 1:500 and added 100 μl per well. and Reaction was allowed at 4° C. for 1 hr. After the reaction, cells were washed in PBS and analyzed using the FACSCalibur device. The negative control group was treated by the FITC-labeled constant region (Fc) specific antibody only. To compare expression degrees of B7-H3 among different cancer cell lines, the value for the peak shift in the experimental groups treated with the B7-H3 monoclonal antibody was divided by the value for the peak shift in the negative control group (MFI Ratio, Mean Fluorescence Intensity Ratio). The result was shown in Table 19 below.

TABLE 19

| | | MFI Ratio | | | | | |
|---|---|---|---|---|---|---|---|
| Cancer Cell Line | | B5 | C4I | D8G | F6V | 10F11 | 84D |
| Ovarian | A2780 | 26.6 | 18.3 | 21.6 | 25.2 | 25.5 | 13.1 |
| Cancer | SKOV-3 | 29.5 | 20.4 | 23.3 | 27.7 | 28.5 | 11.9 |
| | OVCAR-3 | 33.1 | 22.4 | 26.7 | 33.1 | 35.6 | 13.7 |
| Colon | HCT116 | 11.9 | 6.8 | 7.9 | 10.8 | 12 | 5.9 |
| Cancer | HT29 | 17.6 | 12.7 | 11.9 | 15.9 | 18.7 | 8.9 |
| | DLD-1 | 24.9 | 14.9 | 18.5 | 25.6 | 24.6 | 10.7 |
| NSCLC | Calu-6 | 47.9 | 45.5 | 43.2 | N/D | 47.8 | 23.2 |
| TNBC | MDA-MB-231 | 11.4 | 6.2 | 8.5 | 11.3 | 12.4 | 5.4 |
| | MDA-MB-468 | 16.2 | 9.7 | 11.4 | 17.6 | 17.2 | 8.6 |
| Breast | MCF-7 | 154 | 109 | 136 | 151 | 152 | 78 |
| Cancer | HCC1954 | 29 | 21 | 25 | 30 | 35 | 14 |
| | HCC1187 | 21.8 | 11.2 | 13.3 | 18.4 | 22 | 14.9 |
| Kidney | 786-0 | 32 | 24 | 22 | 33 | 34 | 20 |
| Cancer | A-498 | 35 | 26 | 25 | 37 | 35 | 24 |
| Pancreatic | Panc-1 | 18 | 12 | 12 | 19 | 18 | 9 |
| Cancer | | | | | | | |
| Gastric | NCI-N87 | 27.3 | 17.6 | 21.5 | 33.4 | 31.7 | 12.8 |
| Cancer | | | | | | | |
| Cervical | Hela | 38.7 | 26.1 | 30.2 | 42.7 | 40.6 | 23.5 |
| Cancer | | | | | | | |
| MCL | JeKo-1 | 1.5 | 7.9 | 3.1 | N/D | 1.7 | 2.9 |

(MFI Ratio: MFI of anti-B7-H3/MFI of 2nd Ab)
(N/D: not determined)

As the result of measuring the binding capacity using FACS method, it was confirmed that the anti-B7-H3 antibody of the present invention bound to various cancer cell lines derived from ovarian cancer, colorectal cancer, non-small cell lung cancer, breast cancer, renal cancer, pancreatic cancer, gastric cancer, cervical cancer and lymphoma. In addition, it was confirmed that the anti-B7-H3 antibody of the present invention showed higher binding capacity compared to the antibody used as the comparison group, 84D, at the same concentration, and therefore the binding degree to the B7-H3 expressed on a cell surface expression was superior.

2.3.6. Measurement of Binding Capacity to Mouse B7-H3 Antigen of Mouse-Derived Cancer Cell of Anti-B7-H3 Antibody (FACS)

Then, through FACS analysis, the ability of binding to cell surface expression mouse B7-H3 of the anti-B7-H3 antibody of the present invention was measured. It was confirmed that the anti-B7-H3 antibody bound to human B7-H3 and mouse B7-H3 recombinant proteins both through ELISA method in Example 2.3.3. To confirm whether the anti-B7-H3 antibody of the present invention binds to mouse B7-H3 expressed on a cell surface of a mouse cancer cell line, mouse-derived cancer cell lines, CT26 (Mus mesculus colon carcinoma, ATCC® CRL-2638™), B16F10 (Mus musculus skin melanoma, ATCC® CRL-6475™), TC-1 (Mus musulus Lung tumor, ATCC® CRL-2493™) were used.

For each cell line, cells were dissociated and washed with PBS buffer. The number of cells was counted and adjusted to 2×10⁵ cells per well. 200 μl PBS were added. The cells are prepared in a concentration of 10 μg/ml or more in 1% BSA-containing PBS. Each of the anti-B7-H3 antibodies prepared in Example 2.2 and comparison antibody (84D) was reacted with the above-prepared cells at 4° C. for 1 hr.

After washing the cells using PBS buffer, the FITC-labeled anti-human Fc FITC (Sigma, F9512) as diluted by 1:500 were added 100 μl per well, and reaction was allowed at 4° C. for 1 hr. For the control group, only the FITC-labeled anti-human Fc FITC was treated. After washing it twice using PBS buffer again, the degree of binding of the anti-B7-H3 IgG antibodies was measured using FACSCalibur device.

The value for the peak shift in the experimental groups treated with the B7-H3 monoclonal antibody was compared with the value for the peak shift in the negative control group. The result was described in FIG. 11. As the result of measurement using FACS method, it was confirmed that the anti-B7-H3 antibodies of the present invention specifically bound to mouse B7-H3 expressed on a cell surface.

2.4. Measurement of Affinity to B7-H3 of Anti-B7-H3 Antibody

The binding affinity of antigen B7-H3 and anti-B7-H3 antibody was measured by SPR method. First, anti-B7-H3 antibody diluted by 1×HBS-EP buffer was captured with 50 RU on Protein A chip (GE healthcare, Cat. No. 29127556) at a contact time of 60 sec, a stabilization periof of 60 sec and a flow rate of 30 l/min. With 1×HBS-EP buffer, the antigen B7-H3 (R&D systems, 2318-B3-050/CF) was serially two-fold diluted starting from 100 nM to 3.125 nM. At this point, 1×HBS-EP buffer was additionally prepared as a blank. The B7-H3 antigen prepared on the chip in which the anti-B7-H3 antibody was captured was flowed at a flow rate of 30 l/min for the association time of 60 sec and dissociation time of 180 sec. Regeneration was conducted with 10 mM Glycine-HCl pH1.5 (GE healthcare, Cat. No. BR100354) at a flow time of 30 l/min and a contact time of 30 sec. The result was described in Table 20 below.

TABLE 20
Result of measurement of affinity to B7-H3 of anti-B7-H3 antibody

| | Ka | Kd | K_{D} | Rₘₐₓ | |
|---|---|---|---|---|---|
| Ab | (1/Ms, ×10⁵) | (1/s, ×10⁻³) | (M, ×10⁻⁹) | (RU) | Chi² |
| 10F11 | 3.57 | 3.70 | 10.36 | 12.25 | 0.02 |
| B5 | 3.72 | 1.12 | 3.02 | 20.39 | 0.03 |
| C4I | 4.44 | 3.41 | 7.69 | 14.59 | 0.07 |
| D8G | 2.06 | 3.81 | 18.46 | 10.46 | 0.02 |
| F6V | 1.10 | 0.88 | 8.03 | 11.64 | 0.01 |

2.5. Analysis of Anti-Cancer Efficacy by Co-Administration of Anti-B7-H3 Antibody and Anti-PD-1 Antibody in Mouse Isogenic Tumor Transplantation Model

To confirm the efficacy of immune checkpoint inhibition of an antibody in an animal model, a mouse isogenic tumor transplantation model can be used when the antibody has cross-species reactivity between human and mouse.

As confirmed in Examples 2.3.3 and 2.3.6, the anti-B7-H3 antibody of the present invention has cross-species reactivity to a mouse B7-H3 antigen. The inhibition efficacy on tumor proliferation of the anti-B7-H3 antibody of the present invention was confirmed by co-treating it with the anti-mouse PD-1 antibody, RMP1-14 (BioXCell, BE0146) in a mouse isogenic tumor model as follows.

CT26 is a colon carcinoma derived from a mouse (BALB/c) and a cell line overexpressing a mouse B7-H3. It was confirmed that the anti-B7-H3 antibody prepared in Example 2 bound to the mouse B7-H3 expressed on the surface of CT26 mouse cancer cell line in Example 2.3.3-2.3.6 (FIG. 11).

To explain the experimental method in detail, after disassociating CT26 (BALB/c origin) cell line and washing it with PBS buffer, the number of cells was counted and adjusted to 5×10⁵ cells per well. The prepared cells were administered by subcutaneous injection into a mouse (BALB/c, 6-week old, Samtako), and when the size of tumor was 50-100 mm³, the antibodies were administered by 200 jig each, five times at a 3-day interval, a total of 1 mg. The respective tumor sizes for the control group, anti-PD-1 (RMP1-14) antibody single treatment group, and anti-PD-1 antibody and anti-B7-H3 antibody co-treatment group were calculated using a caliper by measuring the longest diameter of tumor (D1) and the diameter vertical to it (D2), to get the volume (0.5*D1*D2²) (FIG. 15).

When the size of tumor was bigger than 2000 mm³ or an ulcer was occurred during tumor observation, the corresponding mice were sacrificed. The survival rate and size of tumor were measured during a total of 30 day observation period after antibody administration was completed.

The result was shown in FIG. 15. As a result, compared to the group in which the anti-PD-1 (RMP1-14) antibody was treated alone, the tumor proliferation inhibition effect and enhancement of survival rate were confirmed in the group in which the anti-PD-1 and anti-B7-H3 antibody were co-administered. The result means that the anti-cancer therapeutic effect is intensified, when the anti-B7-H3 antibody of the present invention showing the immune checkpoint inhibitory efficacy and the anti-PD-1 antibody activating an immunocyte through a different mechanism as another immune inhibitor. As can be confirmed in Example 2.3.3, the binding capacity to mouse B7-H3 of the anti-B7-H3 antibody of the present invention is relatively low compared to human B7-H3. Despite of low binding capacity to mouse B7-H3, the anti-B7-H3 antibody of the present invention showed distinct cancer growth inhibition efficacy and enhancement of survival rate in co-administration with the anti-PD-1 antibody in an isogenic tumor transplantation model, compared to single administration of the anti-PD-1 antibody. The anti-B7-H3 antibody of the present invention is expected to have a stronger immune checkpoint inhibitory effect in human, by stronger binding to human B7-H3, than the result in the mouse isogenic tumor transplantation model.

2.6. Analysis of Tumor-Infiltrating Lymphocyte (TIL) Change by Co-Administration of Anti-B7-H3 Antibody and Anti-PD-1 Antibody in Mouse Isogenic Tumor Transplantation Model

Tumor-infiltrating lymphocytes (TIL) refer to white blood cells which leave bloodstream and move toward tumor. The tumor-infiltrating lymphocytes can comprise a T cell and a B cell, and include mononuclear and polymorphous nuclear immunocytes, are varying depending on the types and stages of tumor, and are related to disease prognosis. In particular, the mechanism of an immune anti-cancer antibody can be investigated through analysis of tumor-infiltrating lymphocytes.

To analyze an anti-cancer effect mechanism by co-administration (combi) of the anti-B7-H3 antibody (F6V) and the anti-PD-1 antibody (RMP-14-1), tumor-infiltrating lymphocytes were analyzed. The experiment was carried out by the same method as Example 2.8. The tumor was isolated from the mouse after 3 times of administration of each antibody was completed, to obtain the tumor-infiltrating lymphocytes.

The tumor infiltrating cells harvested were restimulated with PMA 50 ng/ml and lonomycine 1 μM, and the changes in immunocytes were analyzed (FIG. 16). The representative immunocytes playing a major role of anti-cancer immunoreaction are a cytotoxic T cell and a regulatory T cell. As the result of experiment, in tumor-infiltrating lymphocytes isolated from the mouse in which the anti-B7-H3 antibody and the anti-PD-1 antibody of the present invention were co-administered, the activation of the cytotoxic T cell and proliferation inhibition of the regulatory T cell were clearly observed.

In the tumor-infiltrating lymphocytes isolated from the mouse in which the anti-B7-H3 antibody and anti-PD-1 antibody of the present invention were co-administered, the levels of IFNγ+Granzyme B+ among CD8+ T cells was significantly increased, and the increase in the release of Granzyme B among CD8+ T cells was observed.

In the tumor-infiltrating lymphocytes isolated from the mouse in which the anti-B7-H3 antibody and anti-PD-1 antibody of the present invention were co-administered, the frequency of regulatory T cell and the number of cells were confirmed by using an anti-Foxp3 antibody (eBioscience, FJK-16s), and the proliferative capacity of regulatory T cell was confirmed by using an anti-Ki67 (BD, B56) antibody.

The result was described in FIG. 16. As the result of experiment, it was confirmed that in the group in which the anti-B7-H3 antibody and anti-PD-1 were co-administered, not only the number of regulatory T cells was decreased, but also the Ki67+ frequency showing the proliferative capacity of the regulatory T cell was reduced. Such a result means that the co-administration of the anti-B7-H3 antibody and anti-PD-1 antibody induces increase of activity of the cytotoxic T cell and inhibition of the regulatory T cell at the same time, thereby showing an anti-cancer effect through immune activation.

### Example 3. Preparation of Anti-PD-L1/Anti-B7-H3 Bispecific Antibodies

The anti-PD-L1 B6 and B12 clones prepared in Example 1, and B5 and C4I clones among the anti-B7-H3 clones prepared in Example 2 were exemplarily selected, to prepare anti-PD-L1/anti-B7-H3 bispecific antibodies (PD-L1×B7-H3 bispecific antibodies) in forms of a full-length IgG X scFv and a (HC+LC) X scFab-Fc. The anti-PD-L1 B6 and B12 clones comprise kappa type light chain.

3.1. IgG X scFv Form (2+2 Format) Bispecific Antibody

For the preparation of a 2+2 format bispecific antibody, when PD-L1 or B7-H3 is placed in full IgG part, IgG1 was used.

A DNA segment I having a nucleotide sequence encoding a heavy chain of an IgG antibody of the PD-L1×B7-H3 bispecific antibody was inserted into pcDNA 3.4 (Invitrogen, A14697; plasmid 1), and a DNA segment 2 having a nucleotide sequence encoding a light chain of an IgG antibody of the PD-L1×B7-H3 bispecific antibody was inserted into pcDNA 3.4 (Invitrogen, A14697; plasmid 2). Thereafter, a DNA segment 3 encoding a scFv was fused at a part of the DNA segment 1 corresponding to the c-terminus of the Fc region of the IgG antibody inserted into the plasmid 1, using a DNA segment 4 encoding a linker peptide having 15 amino acid lengths consisting of (GGGGS)3, to construct vectors for the expression of bispecific antibodies. Furthermore, in order to stabilize scFv, additional modification was applied to generate disulfide bridge fusing VL100-VH44 to variable light chain and variable heavy chain, respectively. In otherword, amino acids at V100 and VH44 of scFv were mutated to cysteine.

The sequences of the heavy chain, light chain, scFv and DNA segments are summarized in Tables 21 to 24 below: (bold indicates CDR)

TABLE 21
ABLPNB.01: bispecific antibody comprising the anti-B7-H3 B5 clone in IgG form
and the anti-PD-L1 B6 clone in scFv form

| | | | Amino acid sequence (SEQ ID No.) |
|---|---|---|---|
| Heavy | Heavy chain of | | EVQLLESGGGLVQPGGSLRLSCAASGFTFSDYAMSWVRQAPGKGL |
| Component | B5 | | EWVSSISSGSGSIYYADSVKGRFTISRDNSKNTLYLQMNSLRAEDT |
| | | | AVYYCAKNLIPLDYWGQGTLVTVSSASTKGPSVFPLAPSSKSTSGG |
| | | | TAALGCLVKDYFPEPVTVSWNSGALTSGVHTFPAVLQSSGLYSLSS |
| | | | VVTVPSSSLGTQTYICNVNHKPSNTKVDKKVEPKSCDKTHTCPPCP |
| | | | APELLGGPSVFLFPPKPKDTLMISRTPEVTCVVVDVSHEDPEVKFN |
| | | | WYVDGVEVHNAKTKPREEQYNSTYRVVSVLTVLHQDWLNGKEY |
| | | | KCKVSNKALPAPIEKTISKAKGQPREPQVYTLPPSREEMTKNQVSLT |
| | | | CLVKGFYPSDIAVEWESNGQPENNYKTTPPVLDSDGSFFLYSKLTV |
| | | | DKSRWQQGNVFSCSVMHEALHNHYTQKSLSLSPGK (SEQ ID NO. |
| | | | 165) |
| | Linker | | GGGGSGGGGSGGGGS (SEQ ID NO. 166) |
| | scFv | VL | DIQMTQSPSSLSASVGDRVTITCKASQDVTPAVAWYQQKPGKAPK |
| | of | | LLIYSTSSRYTGVPSRFSGSGSGTDFTFTISSLQPEDIATYYCQQHYT |
| | B6 | | TPLTFGCGTKLEIKR (SEQ ID NO. 167) |
| | | Linker | GGGGSGGGGSGGGGSGGGGS (SEQ ID NO. 168) |
| | | VH | EVQLVESGGGLVQPGGSLRLSCAASGFTFSSYDMSWVRQAPGKCL |
| | | | EWVATISDAGGYIYYRDSVKGRFTISRDNAKNSLYLQMNSLRDED |
| | | | TAVYICARELPWRYALDYWGQGTTVTVSS (SEQ ID NO. 169) |

| | | |
|---|---|---|
| Light | Light chain of | QSVLTQPPSASGTPGQRVTISCSGSSSNIGSNAVSWYQQLPGTAPKL |
| Component | B5 | LIYYNSHRPSGVPDRFSGSKSGTSASLAISGLRSEDEADYYCGSWD |
| | | ASLNAYVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPAECS (SEQ ID NO. 170) |

| | SEQ ID No. |
|---|---|
| Heavy | SEQ ID NO. 171 |
| Component | |
| Light | SEQ ID NO. 172 |
| Component | |

TABLE 22
ABLPNB.02: bispecific antibody comprising the anti-B7-H3 C4I clone in IgG form
and the anti-PD-L1 B6 clone in scFv form

| | | | Amino acid sequence (SEQ ID No.) |
|---|---|---|---|
| Heavy | Heavy chain of | | EVQLLESGGGLVQPGGSLRLSCAASGFTFSGYYMSWVRQAPGKGL |
| Component | C4I | | EWVSLISPSSGSIYYADSVKGRFTISRDNSKNTLYLQMNSLRAEDT |
| | | | AVYYCAKGLTKFDYWGQGTLVTVSSASTKGPSVFPLAPSSKSTSG |
| | | | GTAALGCLVKDYFPEPVTVSWNSGALTSGVHTFPAVLQSSGLYSLS |
| | | | SVVTVPSSSLGTQTYICNVNHKPSNTKVDKKVEPKSCDKTHTCPPC |
| | | | PAPELLGGPSVFLFPPKPKDTLMISRTPEVTCVVVDVSHEDPEVKFN |
| | | | WYVDGVEVHNAKTKPREEQYNSTYRVVSVLTVLHQDWLNGKEY |
| | | | KCKVSNKALPAPIEKTISKAKGQPREPQVYTLPPSREEMTKNQVSLT |
| | | | CLVKGFYPSDIAVEWESNGQPENNYKTTPPVLDSDGSFFLYSKLTV |
| | | | DKSRWQQGNVFSCSVMHEALHNHYTQKSLSLSPGK (SEQ ID NO. |
| | | | 173) |
| | Linker | | GGGGSGGGGSGGGGS (SEQ ID NO. 174) |
| | scFv | VL | DIQMTQSPSSLSASVGDRVTITCKASQDVTPAVAWYQQKPGKAPK |
| | of | | LLIYSTSSRYTGVPSRFSGSGSGTDFTFTISSLQPEDIATYYCQQHYT |
| | B6 | | TPLTFGCGTKLEIKR (SEQ ID NO. 175) |
| | | Linker | GGGGSGGGGSGGGGSGGGGS (SEQ ID NO. 176) |
| | | VH | EVQLVESGGGLVQPGGSLRLSCAASGFTFSSYDMSWVRQAPGKCL |
| | | | EWVATISDAGGYIYYRDSVKGRFTISRDNAKNSLYLQMNSLRDED |
| | | | TAVYICARELPWRYALDYWGQGTTVTVSS (SEQ ID NO. 177) |

| | | |
|---|---|---|
| Light | Light chain of | QSVLTQPPSASGTPGQRVTISCTGSSSNIGSNDVSWYQQLPGTAPKL |
| Component | C4I | LIYANSHRPSGVPDRFSGSKSGTSASLAISGLRSEDEADYYCGSWD |
| | | DSLSGYVFGGGTKLTVLGQPKAAPSVTLFPPSSEELQANKATLVCLI |
| | | SDFYPGAVTVAWKADSSPVKAGVETTTPSKQSNNKYAASSYLSLTP |
| | | EQWKSHRSYSCQVTHEGSTVEKTVAPAECS (SEQ ID NO. 178) |

| | SEQ ID No. |
|---|---|
| Heavy | SEQ ID NO. 179 |
| Component | |
| Light | SEQ ID NO. 180 |
| Component | |

TABLE 23
ABLPNB.03: bispecific antibody comprising the anti-PD-L1 B6 clone in IgG form
and the anti-B7-H3 B5 clone in scFv form

| | | | Amino acid sequence (SEQ ID No.) |
|---|---|---|---|
| Heavy | Heavy chain of | | EVQLVESGGGLVQPGGSLRLSCAASGFTFSSYDMSWVRQAPGKSL |
| Component | B6 | | EWVATISDAGGYIYYRDSVKGRFTISRDNAKNSLYLQMNSLRDED |
| | | | TAVYICARELPWRYALDYWGQGTTVTVSSASTKGPSVFPLAPSSK |
| | | | STSGGTAALGCLVKDYFPEPVTVSWNSGALTSGVHTFPAVLQSSGL |
| | | | YSLSSVVTVPSSSLGTQTYICNVNHKPSNTKVDKKVEPKSCDKTHT |
| | | | CPPCPAPELLGGPSVFLFPPKPKDTLMISRTPEVTCVVVDVSHEDPE |
| | | | VKFNWYVDGVEVHNAKTKPREEQYASTYRVVSVLTVLHQDWLN |
| | | | GKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYS |
| | | | KLTVDKSRWQQGNVFSCSVMHEALHNHYTQKSLSLSPGK (SEQ ID |
| | | | NO. 181) |
| | Linker | | GGGGSGGGGSGGGGS (SEQ ID NO. 182) |
| | scFv | VL | QSVLTQPPSASGTPGQRVTISCSGSSSNIGSNAVSWYQQLPGTAPKL |
| | | | LIYYNSHRPSGVPDRFSGSKSGTSASLAISGLRSEDEADYYCGSWD |
| | | | ASLNAYVFGCGTKLTVL (SEQ ID NO. 183) |
| | of | Linker | GGGGSGGGGSGGGGSGGGGS (SEQ ID NO. 184) |
| | B5 | VH | EVQLLESGGGLVQPGGSLRLSCAASGFTFSDYAMSWVRQAPGKCL |
| | | | EWVSSISSGSGSIYYADSVKGRFTISRDNSKNTLYLQMNSLRAEDT |
| | | | AVYYCAKNLIPLDYWGQGTLVTVSS (SEQ ID NO. 185) |

| | | |
|---|---|---|
| Light | Light chain of | DIQMTQSPSSLSASVGDRVTITCKASQDVTPAVAWYQQKPGKAPK |
| Component | B6 | LLIYSTSSRYTGVPSRFSGSGSGTDFTFTISSLQPEDIATYYCQQHYT |
| | | TPLTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC (SEQ ID NO. 186) |

| | SEQ ID No. |
|---|---|
| Heavy | SEQ ID NO. 187 |
| Component | |
| Light | SEQ ID NO. 188 |
| Component | |

TABLE 24
ABLPNB.04: bispecific antibody comprising the anti-PD-L1 B6 clone in IgG form
and the anti-B7-H3 C4I clone in scFv form

| | | | Amino acid sequence (SEQ ID No.) |
|---|---|---|---|
| Heavy | Heavy chain of | | EVQLVESGGGLVQPGGSLRLSCAASGFTFSSYDMSWVRQAPGKSL |
| Component | B6 | | EWVATISDAGGYIYYRDSVKGRFTISRDNAKNSLYLQMNSLRDED |
| | | | TAVYICARELPWRYALDYWGQGTTVTVSSASTKGPSVFPLAPSSK |
| | | | STSGGTAALGCLVKDYFPEPVTVSWNSGALTSGVHTFPAVLQSSGL |
| | | | YSLSSVVTVPSSSLGTQTYICNVNHKPSNTKVDKKVEPKSCDKTHT |
| | | | CPPCPAPELLGGPSVFLFPPKPKDTLMISRTPEVTCVVVDVSHEDPE |
| | | | VKFNWYVDGVEVHNAKTKPREEQYASTYRVVSVLTVLHQDWLN |
| | | | GKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYS |
| | | | KLTVDKSRWQQGNVFSCSVMHEALHNHYTQKSLSLSPGK (SEQ ID |
| | | | NO. 189) |
| | Linker | | GGGGSGGGGSGGGGS (SEQ ID NO. 190) |
| | scFv | VL | QSVLTQPPSASGTPGQRVTISCTGSSSNIGSNDVSWYQQLPGTAPKL |
| | of | | LIYANSHRPSGVPDRFSGSKSGTSASLAISGLRSEDEADYYCGSWD |
| | C4I | | DSLSGYVFGCGTKLTVL (SEQ ID NO. 191) |
| | | Linker | GGGGSGGGGSGGGGSGGGGS (SEQ ID NO. 192) |
| | | VH | EVQLLESGGGLVQPGGSLRLSCAASGFTFSGYYMSWVRQAPGKCL |
| | | | EWVSLISPSSGSIYYADSVKGRFTISRDNSKNTLYLQMNSLRAEDT |
| | | | AVYYCAKGLTKFDYWGQGTLVTVSS (SEQ ID NO. 193) |

| | | |
|---|---|---|
| Light | Light chain of | DIQMTQSPSSLSASVGDRVTITCKASQDVTPAVAWYQQKPGKAPK |
| Component | B6 | LLIYSTSSRYTGVPSRFSGSGSGTDFTFTISSLQPEDIATYYCQQHYT |
| | | TPLTFGQGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC (SEQ ID NO. 194) |

| | SEQ ID No. |
|---|---|
| Heavy | SEQ ID NO. 195 |
| Component | |
| Light | SEQ ID NO. 196 |
| Component | |

3.2. (HC+LC) X scFab-Fc Form (1+1 Format) Bispecific Antibody

An 1+1 format bispecific antibody was prepared based on a human IgG1 isotype. The PD-L1×B7-H3 bispecific antibody comprises Fc region and two binding arms. One arm comprises a typical light chain and heavy chain. The other arm comprises a single chain Fab-fragment (scFab) in which the C-terminus of a light chain is attached to the N-terminus of VH domain via (GS)9 (G4S)6 (GS)8 linker (64 amino acids length). The C-terminus of the scFab is linked to the N-terminus of Fc domain so as to form a scFab-Fc structure. The scFab-Fc structure therefore comprises a heavy chain, and a light chain which is linked to the N-terminus of the heavy chain via linker. The counterpart of the scFab-Fc structure is a typical heavy chain (HC)+light chain (LC) structure (see FIG. 1b).

Heterodimerization of the two heavy chains of 1+1 format bispecific antibody was achieved by application of the knobs-into-hole technology. The knob mutation (T366W) was introduced into the CH3 domain of the heavy chain, and three mutations to form a hole (T366S, L368A, and Y407V) were introduced into the CH3 domain of the scFab-Fc.

A DNA segment 1 having a nucleotide sequence encoding a typical IgG heavy chain of the bispecific antibody was inserted into pcDNA 3.4 (Invitrogen, A14697; plasmid 1), and a DNA segment 2 having a nucleotide sequence encoding a typical IgG light chain of the bispecific antibody was inserted into pcDNA 3.4 (Invitrogen, A14697; plasmid 2). A DNA segment 3 having a nucleotide sequence encoding a scFab-Fc structure of the bispecific antibody was inserted into pcDNA3.4 (Invitrogen, A14697; plasmid 3).

The sequences of the heavy chain, light chain, scFab-Fc and DNA segments are summarized in Tables 25 to 29 below: (bold indicates CDR)

TABLE 25
ABLPNB.05: bispecific antibody comprising the anti-B7-H3 B5 clone in (HC + LC)
form and the anti-PD-L1 B6 clone in scFab-Fc form

| | | Amino acid sequence (SEQ ID No.) |
|---|---|---|
| (HC + LC) | Heavy | EVQLLESGGGLVQPGGSLRLSCAASGFTFSDYAMSWVRQAPGKGLEWVSSIS |
| of B5 | chain | SGSGSIYYADSVKGRFTISRDNSKNTLYLQMNSLRAEDTAVYYCAKNLIPLD |
| | | YWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQVSL |
| | | WCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO. 197) |
| | Light | QSVLTQPPSASGTPGQRVTISCSGSSSNIGSNAVSWYQQLPGTAPKLLIYYNSH |
| | chain | RPSGVPDRFSGSKSGTSASLAISGLRSEDEADYYCGSWDASLNAYVFGGGTK |
| | | LTVLGQPKAAPSVTLFPPSSEELQANKATLVCLISDFYPGAVTVAWKADSSPV |
| | | KAGVETTTPSKQSNNKYAASSYLSLTPEQWKSHRSYSCQVTHEGSTVEKTVA |
| | | PAECS (SEQ ID NO. 198) |
| scFab-Fc | Whole | DIQMTQSPSSLSASVGDRVTITCKASQDVTPAVAWYQQKPGKAPKLLIYSTSS |
| of B6 | Seq | RYTGVPSRFSGSGSGTDFTFTISSLQPEDIATYYCQQHYTTPLTFGQGTKLEIK |
| | | RTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNR |
| | | GECGSGSGSGSGSGSGSGSGSGGGGSGGGGSGGGGSGGGGSGGGGSGGGGS |
| | | GSGSGSGSGSGSGSGSEVQLVESGGGLVQPGGSLRLSCAASGFTFSSYDMSW |
| | | VRQAPGKSLEWVATISDAGGYIYYRDSVKGRFTISRDNAKNSLYLQMNSLR |
| | | DEDTAVYICARELPWRYALDYWGQGTTVTVSSASTKGPSVFPLAPSSKSTSG |
| | | GTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREP |
| | | QVYTLPPSREEMTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| | | (SEQ ID NO. 199) |
| | VL | DIQMTQSPSSLSASVGDRVTITCKASQDVTPAVAWYQQKPGKAPKLLIYSTSS |
| | | RYTGVPSRFSGSGSGTDFTFTISSLQPEDIATYYCQQHYTTPLTFGQGTKLEIK |
| | | R (SEQ ID NO. 200) |
| | Linker | GSGSGSGSGSGSGSGSGSGGGGSGGGGSGGGGSGGGGSGGGGSGGGGSGSG |
| | | SGSGSGSGSGSGS (SEQ ID NO. 201) |
| | VH | EVQLVESGGGLVQPGGSLRLSCAASGFTFSSYDMSWVRQAPGKSLEWVATIS |
| | | DAGGYIYYRDSVKGRFTISRDNAKNSLYLQMNSLRDEDTAVYICARELPWR |
| | | YALDYWGQGTTVTVSS (SEQ ID NO. 202) |
| | | SEQ ID No. of Nucleotide sequence |
| (HC + LC) | Heavy | SEQ ID NO. 203 |
| of B5 | chain | |
| | Light | SEQ ID NO. 204 |
| | chain | |

| | |
|---|---|
| scFab-Fc of B6 | SEQ ID NO. 205 |

TABLE 26
ABLPNB.06: bispecific antibody comprising the anti-B7-H3 B5 clone in (HC + LC) form
and the anti-PD-L1 B12 clone in scFab-Fc form

| | | Amino acid sequence (SEQ ID No.) |
|---|---|---|
| (HC + LC) | Heavy | EVQLLESGGGLVQPGGSLRLSCAASGFTFSDYAMSWVRQAPGKGLEWVSSIS |
| of B5 | chain | SGSGSIYYADSVKGRFTISRDNSKNTLYLQMNSLRAEDTAVYYCAKNLIPLD |
| | | YWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQVSL |
| | | WCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO. 206) |
| | Light | QSVLTQPPSASGTPGQRVTISCSGSSSNIGSNAVSWYQQLPGTAPKLLIYYNSH |
| | chain | RPSGVPDRFSGSKSGTSASLAISGLRSEDEADYYCGSWDASLNAYVFGGGTK |
| | | LTVLGQPKAAPSVTLFPPSSEELQANKATLVCLISDFYPGAVTVAWKADSSPV |
| | | KAGVETTTPSKQSNNKYAASSYLSLTPEQWKSHRSYSCQVTHEGSTVEKTVA |
| | | PAECS (SEQ ID NO. 207) |
| scFab-Fc | Whole | DIQMTQSPSTLSASVGDRVIITCRASRGISSWLAWYQQKPGKAPNLLISKASSL |
| of B12 | Seq | ESGVPSRFSGSGSGTDFTLTISSLQPEDFATYYCQQSSSIPLTFGGGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGE |
| | | CGSGSGSGSGSGSGSGSGSGGGGSGGGGSGGGGSGGGGSGGGGSGGGGSGS |
| | | GSGSGSGSGSGSGSQVQLLESGGGLVQPGGSLRLSCAASGFTFSSYWMSWVR |
| | | QAPGKGLEWVANIKQDGSEKYYVDSVKGRFTISRDNAKNSLYLQMNSLRAE |
| | | DTAVYYCARVALWDDAFDIWGQGTMVTVSSASTKGPSVFPLAPSSKSTSGGT |
| | | AALGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQV |
| | | YTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK (SEQ |
| | | ID NO. 208) |
| | VL | DIQMTQSPSTLSASVGDRVIITCRASRGISSWLAWYQQKPGKAPNLLISKASS |
| | | LESGVPSRFSGSGSGTDFTLTISSLQPEDFATYYCQQSSSIPLTFGGGTKVEIK |
| | | (SEQ ID NO. 209) |
| | Linker | GSGSGSGSGSGSGSGSGSGGGGSGGGGSGGGGSGGGGSGGGGSGGGGSGSG |
| | | SGSGSGSGSGSGS (SEQ ID NO. 210) |
| | VH | QVQLLESGGGLVQPGGSLRLSCAASGFTFSSYWMSWVRQAPGKGLEWVANI |
| | | KQDGSEKYYVDSVKGRFTISRDNAKNSLYLQMNSLRAEDTAVYYCARVAL |
| | | WDDAFDIWGQGTMVTVSS (SEQ ID NO. 211) |
| | | SEQ ID No. of Nucleotide sequence |
| (HC + LC) | Heavy | SEQ ID NO. 212 |
| | chain | |
| | Light | SEQ ID NO. 213 |
| | chain | |

| | |
|---|---|
| scFab-Fc of B12 | SEQ ID NO. 214 |

TABLE 27
ABLPNB.07: bispecific antibody comprising the anti-B7-H3 C4I clone in (HC + LC) form
and the anti-PD-L1 B6 clone in scFab-Fc form

| | | Amino acid sequence (SEQ ID No.) |
|---|---|---|
| (HC + LC) | Heavy | EVQLLESGGGLVQPGGSLRLSCAASGFTFSGYYMSWVRQAPGKGLEWVSLI |
| of C4I | chain | SPSSGSIYYADSVKGRFTISRDNSKNTLYLQMNSLRAEDTAVYYCAKGLTKF |
| | | DYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQVSL |
| | | WCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO. 215) |
| | Light | QSVLTQPPSASGTPGQRVTISCTGSSSNIGSNDVSWYQQLPGTAPKLLIYANS |
| | chain | HRPSGVPDRFSGSKSGTSASLAISGLRSEDEADYYCGSWDDSLSGYVFGGGT |
| | | KLTVLGQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTVEKTV |
| | | APAECS (SEQ ID NO. 216) |
| scFab-Fc | Whole | DIQMTQSPSSLSASVGDRVTITCKASQDVTPAVAWYQQKPGKAPKLLIYSTSS |
| of B6 | Seq | RYTGVPSRFSGSGSGTDFTFTISSLQPEDIATYYCQQHYTTPLTFGQGTKLEIK |
| | | RTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNR |
| | | GECGSGSGSGSGSGSGSGSGSGGGGSGGGGSGGGGSGGGGSGGGGSGGGGS |
| | | GSGSGSGSGSGSGSGSEVQLVESGGGLVQPGGSLRLSCAASGFTFSSYDMSW |
| | | VRQAPGKSLEWVATISDAGGYIYYRDSVKGRFTISRDNAKNSLYLQMNSLR |
| | | DEDTAVYICARELPWRYALDYWGQGTTVTVSSASTKGPSVFPLAPSSKSTSG |
| | | GTAALGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREP |
| | | QVYTLPPSREEMTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| | | (SEQ ID NO. 217) |
| | VL | DIQMTQSPSSLSASVGDRVTITCKASQDVTPAVAWYQQKPGKAPKLLIYSTSS |
| | | RYTGVPSRFSGSGSGTDFTFTISSLQPEDIATYYCQQHYTTPLTFGQGTKLEIK |
| | | R (SEQ ID NO. 218) |
| | Linker | GSGSGSGSGSGSGSGSGSGGGGSGGGGSGGGGSGGGGSGGGGSGGGGSGSG |
| | | SGSGSGSGSGSGS (SEQ ID NO. 219) |
| | VH | EVQLVESGGGLVQPGGSLRLSCAASGFTFSSYDMSWVRQAPGKSLEWVATIS |
| | | DAGGYIYYRDSVKGRFTISRDNAKNSLYLQMNSLRDEDTAVYICARELPWR |
| | | YALDYWGQGTTVTVSS (SEQ ID NO. 220) |
| | | SEQ ID No. of Nucleotide sequence |
| (HC + LC) | Heavy | SEQ ID NO. 221 |
| of C4I | chain | |
| | Light | SEQ ID NO. 222 |
| | chain | |
| scFab-Fc | | SEQ ID NO. 223 |
| of B6 | | |

TABLE 28
ABLPNB.08: bispecific antibody comprising the anti-B7-H3 C4I clone in (HC + LC) form
and the anti-PD-L1 B12 clone in scFab-Fc form

| | | Amino acid sequence (SEQ ID No.) |
|---|---|---|
| (HC + LC) | Heavy | EVQLLESGGGLVQPGGSLRLSCAASGFTFSGYYMSWVRQAPGKGLEWVSLI |
| of C4I | chain | SPSSGSIYYADSVKGRFTISRDNSKNTLYLQMNSLRAEDTAVYYCAKGLTKF |
| | | DYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDW |
| | | LNGKEYKCKVSNKALPAPIEKTISKAKGQPREPQVYTLPPSREEMTKNQVSL |
| | | WCLVKGFYPSDIAVEWESNGQPENNYKTTPPVLDSDGSFFLYSKLTVDKSRW |
| | | QQGNVFSCSVMHEALHNHYTQKSLSLSPGK (SEQ ID NO. 224) |
| | Light | QSVLTQPPSASGTPGQRVTISCTGSSSNIGSNDVSWYQQLPGTAPKLLIYANS |
| | chain | HRPSGVPDRFSGSKSGTSASLAISGLRSEDEADYYCGSWDDSLSGYVFGGGT |
| | | KLTVLGQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTVEKTV |
| | | APAECS (SEQ ID NO. 225) |
| scFab-Fc | Whole | DIQMTQSPSTLSASVGDRVIITCRASRGISSWLAWYQQKPGKAPNLLISKASSL |
| of B12 | Seq | ESGVPSRFSGSGSGTDFTLTISSLQPEDFATYYCQQSSSIPLTFGGGTKVEIKRT |
| | | VAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGE |
| | | CGSGSGSGSGSGSGSGSGSGGGGSGGGGSGGGGSGGGGSGGGGSGGGGSGS |
| | | GSGSGSGSGSGSGSQVQLLESGGGLVQPGGSLRLSCAASGFTFSSYWMSWVR |
| | | QAPGKGLEWVANIKQDGSEKYYVDSVKGRFTISRDNAKNSLYLQMNSLRAE |
| | | DTAVYYCARVALWDDAFDIWGQGTMVTVSSASTKGPSVFPLAPSSKSTSGGT |
| | | AALGCLVKDYFPEPVTVSWNSGALTSGVHTFPAVLQSSGLYSLSSVVTVPSSS |
| | | LGTQTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQV |
| | | YTLPPSREEMTKNQVSLSCAVKGFYPSDIAVEWESNGQPENNYKTTPPVLDS |
| | | DGSFFLVSKLTVDKSRWQQGNVFSCSVMHEALHNHYTQKSLSLSPGK (SEQ |
| | | ID NO. 226) |
| | VL | DIQMTQSPSTLSASVGDRVIITCRASRGISSWLAWYQQKPGKAPNLLISKASS |
| | | LESGVPSRFSGSGSGTDFTLTISSLQPEDFATYYCQQSSSIPLTFGGGTKVEIK |
| | | (SEQ ID NO. 227) |
| | Linker | GSGSGSGSGSGSGSGSGSGGGGSGGGGSGGGGSGGGGSGGGGSGGGGSGSG |
| | | SGSGSGSGSGSGS (SEQ ID NO. 228) |
| | VH | QVQLLESGGGLVQPGGSLRLSCAASGFTFSSYWMSWVRQAPGKGLEWVANI |
| | | KQDGSEKYYVDSVKGRFTISRDNAKNSLYLQMNSLRAEDTAVYYCARVAL |
| | | WDDAFDIWGQGTMVTVSS (SEQ ID NO. 229) |
| | | SEQ ID No. of Nucleotide sequence |
| (HC + LC) | Heavy | SEQ ID NO. 230 |
| of C4I | chain | |
| | Light | SEQ ID NO. 231 |
| | chain | |

| | |
|---|---|
| scFab-Fc of B12 | SEQ ID NO. 232 |

TABLE 29
ABLPNB.09: bispecific antibody comprising the anti-PD-L1 B6 clone in (HC + LC) form
and the anti-B7-H3 C4I clone in scFab-Fc form

| | | Amino acid sequence (SEQ ID No.) |
|---|---|---|
| (HC + LC) | Heavy | EVQLVESGGGLVQPGGSLRLSCAASGFTFSSYDMSWVRQAPGKSLEWVATIS |
| of B6 | chain | DAGGYIYYRDSVKGRFTISRDNAKNSLYLQMNSLRDEDTAVYICARELPWR |
| | | YALDYWGQGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPEVT |
| | | CVVVDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSREEMTKNQ |
| | | VSLWCLVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO. 233) |
| | Light | DIQMTQSPSSLSASVGDRVTITCKASQDVTPAVAWYQQKPGKAPKLLIYSTSS |
| | chain | RYTGVPSRFSGSGSGTDFTFTISSLQPEDIATYYCQQHYTTPLTFGQGTKLEIK |
| | | RTVAAPSVFIFPPSDEQLKSGTASVVCLLNNFYPREAKVQWKVDNALQSGNS |
| | | QESVTEQDSKDSTYSLSSTLTLSKADYEKHKVYACEVTHQGLSSPVTKSFNR |
| | | GEC (SEQ ID NO. 234) |
| scFab-Fc | Whole | QSVLTQPPSASGTPGQRVTISCTGSSSNIGSNDVSWYQQLPGTAPKLLIYANS |
| of C4I | Seq | HRPSGVPDRFSGSKSGTSASLAISGLRSEDEADYYCGSWDDSLSGYVFGGGT |
| | | KLTVLGQPKAAPSVTLFPPSSEELQANKATLVCLISDFYPGAVTVAWKADSSP |
| | | VKAGVETTTPSKQSNNKYAASSYLSLTPEQWKSHRSYSCQVTHEGSTVEKTV |
| | | APAECSGSGSGSGSGSGSGSGSGSGGGGSGGGGSGGGGSGGGGSGGGGSGG |
| | | GGSGSGSGSGSGSGSGSGSEVQLLESGGGLVQPGGSLRLSCAASGFTFSGYY |
| | | MSWVRQAPGKGLEWVSLISPSSGSIYYADSVKGRFTISRDNSKNTLYLQMNS |
| | | LRAEDTAVYYCAKGLTKFDYWGQGTLVTVSSASTKGPSVFPLAPSSKSTSGG |
| | | TAALGCLVKDYFPEPVTVSWNSGALTSGVHTFPAVLQSSGLYSLSSVVTVPSS |
| | | SLGTQTYICNVNHKPSNTKVDKKVEPKSCDKTHTCPPCPAPELLGGPSVFLFP |
| | | PKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQ |
| | | VYTLPPSREEMTKNQVSLSCAVKGFYPSDIAVEWESNGQPENNYKTTPPVLD |
| | | SDGSFFLVSKLTVDKSRWQQGNVFSCSVMHEALHNHYTQKSLSLSPGK (SEQ |
| | | ID NO. 235) |
| | VL | QSVLTQPPSASGTPGQRVTISCTGSSSNIGSNDVSWYQQLPGTAPKLLIYANS |
| | | HRPSGVPDRFSGSKSGTSASLAISGLRSEDEADYYCGSWDDSLSGYVFGGGT |
| | | KLTVL (SEQ ID NO. 236) |
| | Linker | GSGSGSGSGSGSGSGSGSGGGGSGGGGSGGGGSGGGGSGGGGSGGGGSGSG |
| | | SGSGSGSGSGSGS (SEQ ID NO. 237) |
| | VH | EVQLLESGGGLVQPGGSLRLSCAASGFTFSGYYMSWVRQAPGKGLEWVSLI |
| | | SPSSGSIYYADSVKGRFTISRDNSKNTLYLQMNSLRAEDTAVYYCAKGLTKF |
| | | DYWGQGTLVTVSS (SEQ ID NO. 238) |
| | | SEQ ID No. of Nucleotide sequence |
| (HC + LC) | Heavy | SEQ ID NO. 239 |
| of B6 | chain | |
| | Light | SEQ ID NO. 240 |
| | chain | |
| | of B6 | |

| | |
|---|---|
| scFab-Fc of C4I | SEQ ID NO. 241 |

3.3. Production and Isolation of Bispecific Antibodies

The constructed vectors were transiently expressed in ExpiCHO-S™ cells (Thermo Fisher, A29127) using (ExpiFectamine™CHO Kit, Thermo, A29129), cultured in ExpiCHO™ Expression medium (Thermo, A29100-01) under the conditions of 30 to 37° C. for 7 to 15 days in a C02 incubator equipped with rotating shaker. Plasmid DNA (250 μg) and ExpiFectamin CHO Reagent (800 μL) were mixed with Opti-MEM® I medium (20 mL final volume) and allowed to stand at room temperature for 5 min. The mixed solution was added to 6×10⁶ ExpiCHO cells cultured in ExpiCHO Expression Medium and gently mixed in a shaker incubator at 37° C. with a humidified atmosphere of 8% C02 in air. At 18 hours post-transfection, 1.5 mL of ExpiFectamin CHO Transfection Enhancer 1 and 60 mL of ExpiFectamin CHO Transfection Feed were added to each flask.

Each BsAb was purified from the cell culture supernatant by recombinant Protein A affinity chromatography (Hitrap Mabselect Sure, GE Healthcare, 28-4082-55) and gel filtration chromatography with a HiLoad 26/200 Superdex200 prep grade column (GE Healthcare, 28-9893-36). SDS-PAGE (NuPage 4-12% Bis-Tris gel, NP0321) and size exclusion HPLC (Agilent, 1200 series) analysis with SE-HPLC column (SWXL SE-HPLC column, TOSOH, G3000SWXL) were performed to detect and confirm the size and purity of each BsAb. Purified proteins were concentrated in PBS by ultrafiltration using a Amicon Ultra 15 30K device (Merck, UFC903096), and protein concentrations were estimated using a nanodrop (Thermo, Nanodrop One). In case of 2+2 format bispecific antibody, when a two-vector system is applied, the ratio between light to heavy chain could be 1:1 to 1:3 by weight. Alternatively, in case of 1+1 format bispecific antibody, three vector system is applied, the ratio between light chain, heavy chain, and scFab-Fc could be 1:1:2 to 1:1:5 by weight.

The prepared PD-L1×B7-H3 bispecific antibodies are as below:

TABLE 30

| | IgG | scFv | Name |
|---|---|---|---|
| IgG × scFv | B7-H3 | PD-L1 | B5xB6 |
| (2 + 2 format) | | | C4IxB6 |
| | PD-L1 | B7-H3 | B6xB5 |
| | | | B6xC4I |

TABLE 31

| | HC + LC | scFab-Fc | Name |
|---|---|---|---|
| (HC + LC) × scFab-Fc | B7-H3 | PD-L1 | B5xB6 |
| (1 + 1 format) | | | B5xB12 |
| | | | C4IxB6 |
| | | | C4IxB12 |
| | PD-L1 | B7-H3 | B6xC4I |

### Example 4. Characterization of Bispecific Antibodies PD-L1×B7-H3

4.1. Cell Binding Assay (FACS) for Format Comparison

To evaluate the antigen binding property, the antibody candidates were analyzed for its binding to mammalian expressed both B7-H3 and PD-L1 by FACS. Briefly, RKO cells were incubated with the bispecific antibodies. After wash by FACS buffer (1% BSA in PBS), the FITC-anti-human IgG antibody was added to each well and incubated at 4° C. for 1 hour. The MFI of FITC was evaluated by FACS Caliber. The results are described in FIG. 17.

As shown in FIG. 17, the tested bispecific antibodies showed binding ability to PD-L1 and B7-H3 which expressed on cell surface and could efficiently bind to PD-L1 and B7-H3 expressed on RKO cells. In addition, 1+1 format bispecific antibodies showed even superior binding potency than 2+2 format bispecific antibodies in both B5×B6 and C4I×B6 clones.

4.2. Cell Binding Assay (FACS) for Clone Comparison

To evaluate the antigen binding property, 1+1 format bispecific antibodies were analyzed for its binding to mammalian expressed both B7-H3 and PD-L1 by FACS as compared to monospecific antibodies. Briefly, RKO cells (human colon carcinoma cell line) were incubated with antibodies. After wash by FACS buffer (1% BSA in PBS), the FITC-anti-human IgG antibody was added to each well and incubated at 4° C. for 1 hour. The MFI of FITC was evaluated by FACS Caliber. The results are described in FIG. 18.

As shown in FIG. 18, all the tested 1+1 format bispecific antibodies showed superior binding affinity than monospecific antibodies.

4.3. Cell Based Functional Assay for Format Comparison

To evaluate the in vitro tumor cell killing potency by IG4 TCR T cells, the antibody candidates were analyzed by IG4 TCR-engineered T cell assay. Specifically, Lentiviral vector for IG4 TCR recognizing the HLA-A*02-restricted melanoma antigen NY-ESO-1 was generated. For transduction, IG4 TCR expressing lentivirus was produced in Lenti-Pac 293Ta cell line (GeneCopoeia) and human T cells were activated by Dyna beads Human T-Activator CD3/CD28 (Gibco). 72 hrs after the activation, human T cells were transduced by IG4 TCR expressing lentivirus and expanded for 7 days with hIL-2.

Luciferase-labeled A2-ESO+ tumor cells were seeded in a flat-bottom 96-well plate at specific density per well in triplicates. After 24 hrs, IG4 TCR-expressing human T cells were co-cultured at the designated effector:target (E:T) ratios in the presence of samples. The plate were incubated for 48 hrs at 37° C. and 5% C02 and the relative luciferase activity was measured by the One-Glo luciferase assay system (Promega) according to the manufacturer's instructions. The results are described in FIG. 19.

As shown in FIG. 19, the tested bispecific antibodies showed better T cell killing potency than monospecific antibodies, and the 1+1 format bispecific antibodies showed even better T cell killing potency than 2+2 format bispecific antibodies.

4.4. Cell Based Functional Assay for Clone Comparison

To evaluate the antibody-dependent cell-mediated cytotoxicity (ADCC), the antibody candidates of 1+1 format were analyzed. The ability of ADCC of anti-PD-L1/anti-B7-H3 bispecific antibodies was confirmed by using ADCC Reporter Bioassay (Promega, G7018) kit. The experiment method was performed in accordance with the protocol of the manufacturer, and RKO cells (B7-H3/PD-L1 positive cell line) and KatoIII cells (B7-H3/PD-L1 negative cell line) were used for the assay. The results thereof are described in FIG. 20.

As shown in FIG. 20, among the 1+1 format bispecific antibodies, C4I×B6 bispecific antibody showed the most superior ADCC activity than other clones, and B5×B6 bispecific antibody was the next.

4.5. Cell Based Functional Assay for Characterization of C4I×B6 and B5×B6

The ability of ADCC of B7-H3×PD-L1 bispecific antibody in 1+1 Format was confirmed by using ADCC Reporter Bioassay (Promega, G7018) kit. The experiment method was performed in accordance with the protocol of the manufacturer, and RKO cells and KatoIII cells were used for the assay.

4.6. Cell Based Functional Assay for Characterization of C4I×B6 and B5×B6

To evaluate the in vitro tumor cell killing potency by IG4 TCR T cells, C4I×B6 and B5×B6 bispecific antibodies in 1+1 format were analyzed by IG4 TCR-engineered T cell assay. Specifically, Lentiviral vector for IG4 TCR recognizing the HLA-A*02-restricted melanoma antigen NY-ESO-1 was generated. For transduction, IG4 TCR expressing lentivirus was produced in Lenti-Pac 293Ta cell line (GeneCopoeia) and human T cells were activated by Dyna beads Human T-Activator CD3/CD28 (Gibco). 72 hrs after the activation, human T cells were transduced by IG4 TCR expressing lentivirus and expanded for 7 days with hIL-2.

Luciferase-labeled A2-ESO+ tumor cells (B7-H3/PD-L1 positive A375-PD-L1 cell line) were seeded in a flat-bottom 96-well plate at specific density per well in triplicates. After 24 hrs, IG4 TCR-expressing human T cells were co-cultured at the designated effector:target (E:T) ratios in the presence of samples. The plate were incubated for 48 hrs at 37° C. and 5% CO₂ and the relative luciferase activity was measured by the One-Glo luciferase assay system (Promega) according to the manufacturer's instructions. The results are described in FIG. 21.

As shown in FIG. 21, C4I×B6 bispecific antibody showed better tumor cell killing potency than B5×B6 bispecific antibody.

4.7. In Vivo Efficacy Test for Characterization of C4I×B6 and B5×B6

To evaluate tumor growth inhibition of the bispecific antibodies in 1+1 format, in vivo efficacy test was performed using RKO-PBMC humanized mice model. Specifically, the NSG mice (6-8 weeks) were purchased from Jackson Laboratory. Each animal was inoculated s.c. into the right lower flank with 5×10⁶ of RKO cells. On the day of grouping, 1×10⁷ cells of human PBMCs (Stem express, USA) were delivered intravenously through the lateral tail vein. Mice were intraperitoneally administered Q3D for 6 times with following antibodies: isotype control (G1, 10 mg/kg), anti-PD-L1 monospecific antibody (G2—B6, 5 mg/kg), anti-B7-H3 monospecific antibody (G3—C4I, 5 mg/kg), combination of anti-PD-L1 (B6, 5 mg/kg) and anti-B7-H3 (C4I, 5 mg/kg) monospecific antibodies (G4), and B7-H3×PD-L1 bispecific antibody (G5—C4I×B6, 10 mg/kg). The results are described in FIG. 22.

As shown in FIG. 22, C4I×B6 bispecific antibody treatment group was the most efficacious among other treatment groups. Bispecific antibody treatment resulted in tumor growth inhibition that is even better than the combination of each monoclonal antibody.

### Example 5. Preparation of Anti-PD-L1/Anti-B7-H3/Anti-4-1BB Trispecific Antibodies

C4I×B6 bispecific antibody and C4I×B12 bispecific antibody prepared in Example 3 were further modified to produce anti-PD-L1/anti-B7-H3/anti-4-1BB trispecific antibodies in a form of (HC+LC) X scFab-Fc X scFv. The trispecific antibody comprise scFv fragment binding to 4-1BB protein further to 1+1 format bispecific antibody, and scFv fragment binding 4-1BB protein (1A10 clone) is linked to the C-terminus of each of Fc domains of the bispecific antibody via linker.

A DNA segment 1 having a nucleotide sequence encoding a typical IgG heavy chain of the bispecific antibody was inserted into pcDNA 3.4 (Invitrogen, A14697; plasmid 1), and a DNA segment 2 having a nucleotide sequence encoding a typical IgG light chain of the bispecific antibody was inserted into pcDNA 3.4 (Invitrogen, A14697; plasmid 2). A DNA segment 3 having a nucleotide sequence encoding a scFab-Fc structure of the bispecific antibody was inserted into pcDNA3.4 (Invitrogen, A14697; plasmid 3). Thereafter, a DNA segment 4 encoding a scFv was fused at a part of the DNA segment 1 and 3 corresponding to the c-terminus of the Fc region of the IgG antibody inserted into the plasmid 1 and 3, using a DNA segment 5 encoding a linker peptide having 15 amino acid lengths consisting of (GGGGS)3, to construct vectors for the expression of trispecific antibodies. Furthermore, in order to stabilize scFv, additional modification was applied to generate disulfide bridge fusing VL100-VH44 to variable light chain and variable heavy chain, respectively. In other word, amino acids at VL100 and VH44 of scFv were mutated to cysteine.

The preparation, production and isolation of the trispecific antibody are conducted according to the methods described in Example 3.

The sequences of the heavy chain, light chain, scFab-Fc, the scFv and DNA segments are summarized in Tables 32 and 33 below:

TABLE 32
Trispecific antibody 01: Trispecific antibody comprising the anti-B7-H3 C4I clone in (HC + LC)
form, the anti-PD-L1 B6 clone in scFab-Fc form and the anti-4-IBB 1A10 in scFv form

| | | Amino acid sequence (Seq ID No.) |
|---|---|---|
| (HC + LC) | Whole | EVQLLESGGGLVQPGGSLRLSCAASGFTFSGYYMSWVRQAPGKGLEWVSL |
| of C4I | Seq: | ISPSSGSIYYADSVKGRFTISRDNSKNTLYLQMNSLRAEDTAVYYCAKGLTK |
| + | HC of | FDYWGQGTLVTVSSASTKGPSVFPLAPSSKSTSGGTAALGCLVKDYFPEPVT |
| scFv of | C4I + | VSWNSGALTSGVHTFPAVLQSSGLYSLSSVVTVPSSSLGTQTYICNVNHKPS |
| 1A10 | scFv of | NTKVDKKVEPKSCDKTHTCPPCPAPELLGGPSVFLFPPKPKDTLMISRTPEV |
| | 1A10 | TCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMT |
| | | KNQVSLWCLVKGFYPSDIAVEWESNGQPENNYKTTPPVLDSDGSFFLYSKL |
| | | TVDKSRWQQGNVFSCSVMHEALHNHYTQKSLSLSPGKGGGGSGGGGSGG |
| | | GGSQSVLTQPPSASGTPGQRVTISCSGSSSNIGNNYVTWYQQLPGTAPKLLIY |
| | | ADSHRPSGVPDRFSGSKSGTSASLAISGLRSEDEADYYCATWDYSLSGYVF |
| | | GCGTKLTVLGGGGSGGGGSGGGGSGGGGSEVQLLESGGGLVQPGGSLRLS |
| | | CAASGFTFSSYDMSWVRQAPGKCLEWVSWISYSGGSIYYADSVKGRFTISR |
| | | DNSKNTLYLQMNSLRAEDTAVYYCARDAQRNSMREFDYWGQGTLVTVSS |
| | | (SEQ ID NO. 300) |
| | Heavy | EVQLLESGGGLVQPGGSLRLSCAASGFTFSGYYMSWVRQAPGKGLEWVSL |
| | chain of | ISPSSGSIYYADSVKGRFTISRDNSKNTLYLQMNSLRAEDTAVYYCAKGLT |
| | C4I | KFDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSREEM |
| | | TKNQVSLWCLVKGFYPSDIAVEWESNGQPENNYKTTPPVLDSDGSFFLYSK |
| | | LTVDKSRWQQGNVFSCSVMHEALHNHYTQKSLSLSPGK (SEQ ID NO. 242) |
| | Linker | GGGGSGGGGSGGGGS (SEQ ID NO. 243) |
| | scFv of | QSVLTQPPSASGTPGQRVTISCSGSSSNIGNNYVTWYQQLPGTAPKLLIYADS |
| | 1A10 | HRPSGVPDRFSGSKSGTSASLAISGLRSEDEADYYCATWDYSLSGYVFGCG |
| | | TKLTVLGGGGSGGGGSGGGGSGGGGSEVQLLESGGGLVQPGGSLRLSCAA |
| | | SGFTFSSYDMSWVRQAPGKCLEWVSWISYSGGSIYYADSVKGRFTISRDNS |
| | | KNTLYLQMNSLRAEDTAVYYCARDAQRNSMREFDYWGQGTLVTVSS (SEQ |
| | | ID NO. 244) |
| | Light | QSVLTQPPSASGTPGQRVTISCTGSSSNIGSNDVSWYQQLPGTAPKLLIYAN |
| | chain of | SHRPSGVPDRFSGSKSGTSASLAISGLRSEDEADYYCGSWDDSLSGYVFGG |
| | C4I | GTKLTVLGQPKAAPSVTLFPPSSEELQANKATLVCLISDFYPGAVTVAWKAD |
| | | SSPVKAGVETTTPSKQSNNKYAASSYLSLTPEQWKSHRSYSCQVTHEGSTV |
| | | EKTVAPAECS (SEQ ID NO. 245) |
| scFab-Fc | Whole | DIQMTQSPSSLSASVGDRVTITCKASQDVTPAVAWYQQKPGKAPKLLIYSTS |
| 1A10 | Seq | SRYTGVPSRFSGSGSGTDFTFTISSLQPEDIATYYCQQHYTTPLTFGQGTKLEI |
| of B6 | | KRTVAAPSVFIFPPSDEQLKSGTASVVCLLNNFYPREAKVQWKVDNALQSG |
| + | | NSQESVTEQDSKDSTYSLSSTLTLSKADYEKHKVYACEVTHQGLSSPVTKSF |
| scFv of | | NRGECGSGSGSGSGSGSGSGSGSGGGGSGGGGSGGGGSGGGGSGGGGSGG |
| 1A10 | | GGSGSGSGSGSGSGSGSGSEVQLVESGGGLVQPGGSLRLSCAASGFTFSSYD |
| | | MSWVRQAPGKSLEWVATISDAGGYIYYRDSVKGRFTISRDNAKNSLYLQM |
| | | NSLRDEDTAVYICARELPWRYALDYWGQGTTVTVSSASTKGPSVFPLAPSS |
| | | KSTSGGTAALGCLVKDYFPEPVTVSWNSGALTSGVHTFPAVLQSSGLYSLSS |
| | | VVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKTIS |
| | | KAKGQPREPQVYTLPPSREEMTKNQVSLSCAVKGFYPSDIAVEWESNGQPE |
| | | NNYKTTPPVLDSDGSFFLVSKLTVDKSRWQQGNVFSCSVMHEALHNHYTQ |
| | | KSLSLSPGKGGGGSGGGGSGGGGSQSVLTQPPSASGTPGQRVTISCSGSSSNI |
| | | GNNYVTWYQQLPGTAPKLLIYADSHRPSGVPDRFSGSKSGTSASLAISGLRS |
| | | EDEADYYCATWDYSLSGYVFGCGTKLTVLGGGGSGGGGSGGGGSGGGGS |
| | | EVQLLESGGGLVQPGGSLRLSCAASGFTFSSYDMSWVRQAPGKCLEWVSW |
| | | ISYSGGSIYYADSVKGRFTISRDNSKNTLYLQMNSLRAEDTAVYYCARDAQ |
| | | RNSMREFDYWGQGTLVTVSS(SEQ ID NO. 246) |
| | Light | DIQMTQSPSSLSASVGDRVTITCKASQDVTPAVAWYQQKPGKAPKLLIYST |
| | chain of | SSRYTGVPSRFSGSGSGTDFTFTISSLQPEDIATYYCQQHYTTPLTFGQGTK |
| | B6 | LEIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC (SEQ ID NO. 247) |
| | Linker | GSGSGSGSGSGSGSGSGSGGGGSGGGGSGGGGSGGGGSGGGGSGGGGSGS |
| | | GSGSGSGSGSGSGS (SEQ ID NO. 248) |
| | Heavy | EVQLVESGGGLVQPGGSLRLSCAASGFTFSSYDMSWVRQAPGKSLEWVATI |
| | chain of | SDAGGYIYYRDSVKGRFTISRDNAKNSLYLQMNSLRDEDTAVYICARELP |
| | B6 | WRYALDYWGQGTTVTVSSASTKGPSVFPLAPSSKSTSGGTAALGCLVKDY |
| | | FPEPVTVSWNSGALTSGVHTFPAVLQSSGLYSLSSVVTVPSSSLGTQTYICNV |
| | | NHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPREEQYNSTYRVV |
| | | SVLTVLHQDWLNGKEYKCKVSNKALPAPIEKTISKAKGQPREPQVYTLPPS |
| | | REEMTKNQVSLSCAVKGFYPSDIAVEWESNGQPENNYKTTPPVLDSDGSFF |
| | | LVSKLTVDKSRWQQGNVFSCSVMHEALHNHYTQKSLSLSPGK (SEQ ID NO. |
| | | 249) |
| | Linker | GGGGSGGGGSGGGGS (SEQ ID NO. 250) |
| | scFv of | QSVLTQPPSASGTPGQRVTISCSGSSSNIGNNYVTWYQQLPGTAPKLLIYADS |
| | 1A10 | HRPSGVPDRFSGSKSGTSASLAISGLRSEDEADYYCATWDYSLSGYVFGCG |
| | | TKLTVLGGGGSGGGGSGGGGSGGGGSEVQLLESGGGLVQPGGSLRLSCAA |
| | | SGFTFSSYDMSWVRQAPGKCLEWVSWISYSGGSIYYADSVKGRFTISRDNS |
| | | KNTLYLQMNSLRAEDTAVYYCARDAQRNSMREFDYWGQGTLVTVSS (SEQ |
| | | ID NO. 251) |
| | | SEQ ID No. of Nucleotide sequence |
| (HC + LC) | Whole | SEQ ID: 301 |
| of C4I | Seq: | |
| + | HC of | |
| scFv of | C4I + | |
| 1A10 | scFv of | |
| | 1A10 | |
| | Heavy | SEQ ID NO. 252 |
| | chain of | |
| | C4I | |
| | Linker | SEQ ID NO. 253 |
| | scFv of | SEQ ID NO. 254 |
| | 1A10 | |
| | Light | SEQ ID NO. 255 |
| | chain of | |
| | C4I | |
| scFab-Fc | Whole | SEQ ID NO. 256 |
| of B6 | Seq | |
| + | Light | SEQ ID NO. 257 |
| scFv of | chain of | |
| 1A10 | B6 | |
| | Linker | SEQ ID NO. 258 |
| | Heavy | SEQ ID NO. 259 |
| | chain of | |
| | B6 | |
| | Linker | SEQ ID NO. 260 |
| | scFv of | SEQ ID NO. 261 |
| | 1A10 | |

TABLE 33
Trispecific antibody 02: Trispecific antibody comprising the anti-B7-H3 C4I clone in (HC + LC)
form, the anti-PD-L1 B12 clone in scFab-Fc form and the anti-4-1BB 1A10 in scFv form

| | | Amino acid sequence (Seq ID No.) |
|---|---|---|
| (HC + LC) | Whole | EVQLLESGGGLVQPGGSLRLSCAASGFTFSGYYMSWVRQAPGKGLEWVSL |
| of C4I | Seq: | ISPSSGSIYYADSVKGRFTISRDNSKNTLYLQMNSLRAEDTAVYYCAKGLTK |
| + | HC of | FDYWGQGTLVTVSSASTKGPSVFPLAPSSKSTSGGTAALGCLVKDYFPEPVT |
| scFv of | C4I + | VSWNSGALTSGVHTFPAVLQSSGLYSLSSVVTVPSSSLGTQTYICNVNHKPS |
| 1A10 | scFv of | NTKVDKKVEPKSCDKTHTCPPCPAPELLGGPSVFLFPPKPKDTLMISRTPEV |
| | 1A10 | TCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSREEMT |
| | | KNQVSLWCLVKGFYPSDIAVEWESNGQPENNYKTTPPVLDSDGSFFLYSKL |
| | | TVDKSRWQQGNVFSCSVMHEALHNHYTQKSLSLSPGKGGGGSGGGGSGG |
| | | GGSQSVLTQPPSASGTPGQRVTISCSGSSSNIGNNYVTWYQQLPGTAPKLLIY |
| | | ADSHRPSGVPDRFSGSKSGTSASLAISGLRSEDEADYYCATWDYSLSGYVF |
| | | GCGTKLTVLGGGGSGGGGSGGGGSGGGGSEVQLLESGGGLVQPGGSLRLS |
| | | CAASGFTFSSYDMSWVRQAPGKCLEWVSWISYSGGSIYYADSVKGRFTISR |
| | | DNSKNTLYLQMNSLRAEDTAVYYCARDAQRNSMREFDYWGQGTLVTVSS |
| | | (SEQ ID NO. 302) |
| | Heavy | EVQLLESGGGLVQPGGSLRLSCAASGFTFSGYYMSWVRQAPGKGLEWVSL |
| | chain of | ISPSSGSIYYADSVKGRFTISRDNSKNTLYLQMNSLRAEDTAVYYCAKGLT |
| | C4I | KFDYWGQGTLVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHK |
| | | PSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPSREEM |
| | | TKNQVSLWCLVKGFYPSDIAVEWESNGQPENNYKTTPPVLDSDGSFFLYSK |
| | | LTVDKSRWQQGNVFSCSVMHEALHNHYTQKSLSLSPGK (SEQ ID NO. 262) |
| | Linker | GGGGSGGGGSGGGGS (SEQ ID NO. 263) |
| | scFv of | QSVLTQPPSASGTPGQRVTISCSGSSSNIGNNYVTWYQQLPGTAPKLLIYADS |
| | 1A10 | HRPSGVPDRFSGSKSGTSASLAISGLRSEDEADYYCATWDYSLSGYVFGCG |
| | | TKLTVLGGGGSGGGGSGGGGSGGGGSEVQLLESGGGLVQPGGSLRLSCAA |
| | | SGFTFSSYDMSWVRQAPGKCLEWVSWISYSGGSIYYADSVKGRFTISRDNS |
| | | KNTLYLQMNSLRAEDTAVYYCARDAQRNSMREFDYWGQGTLVTVSS (SEQ |
| | | ID NO. 264) |
| | Light | QSVLTQPPSASGTPGQRVTISCTGSSSNIGSNDVSWYQQLPGTAPKLLIYAN |
| | chain of | SHRPSGVPDRFSGSKSGTSASLAISGLRSEDEADYYCGSWDDSLSGYVFGG |
| | C4I | GTKLTVLGQPKAAPSVTLFPPSSEELQANKATLVCLISDFYPGAVTVAWKAD |
| | | SSPVKAGVETTTPSKQSNNKYAASSYLSLTPEQWKSHRSYSCQVTHEGSTV |
| | | EKTVAPAECS (SEQ ID NO. 265) |
| scFab-Fc | Whole | DIQMTQSPSTLSASVGDRVIITCRASRGISSWLAWYQQKPGKAPNLLISKASS |
| of B12 | Seq | LESGVPSRFSGSGSGTDFTLTISSLQPEDFATYYCQQSSSIPLTFGGGTKVEIK |
| + | | RTVAAPSVFIFPPSDEQLKSGTASVVCLLNNFYPREAKVQWKVDNALQSGN |
| scFv of | | SQESVTEQDSKDSTYSLSSTLTLSKADYEKHKVYACEVTHQGLSSPVTKSFN |
| 1A10 | | RGECGSGSGSGSGSGSGSGSGSGGGGSGGGGSGGGGSGGGGSGGGGSGGG |
| | | GSGSGSGSGSGSGSGSGSQVQLLESGGGLVQPGGSLRLSCAASGFTFSSYW |
| | | MSWVRQAPGKGLEWVANIKQDGSEKYYVDSVKGRFTISRDNAKNSLYLQ |
| | | MNSLRAEDTAVYYCARVALWDDAFDIWGQGTMVTVSSASTKGPSVFPLAP |
| | | SSKSTSGGTAALGCLVKDYFPEPVTVSWNSGALTSGVHTFPAVLQSSGLYSL |
| | | SSVVTVPSSSLGTQTYICNVNHKPSNTKVDKKVEPKSCDKTHTCPPCPAPEL |
| | | LGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTI |
| | | SKAKGQPREPQVYTLPPSREEMTKNQVSLSCAVKGFYPSDIAVEWESNGQP |
| | | ENNYKTTPPVLDSDGSFFLVSKLTVDKSRWQQGNVFSCSVMHEALHNHYT |
| | | QKSLSLSPGKGGGGSGGGGSGGGGSQSVLTQPPSASGTPGQRVTISCSGSSS |
| | | NIGNNYVTWYQQLPGTAPKLLIYADSHRPSGVPDRFSGSKSGTSASLAISGL |
| | | RSEDEADYYCATWDYSLSGYVFGCGTKLTVLGGGGSGGGGSGGGGSGGG |
| | | GSEVQLLESGGGLVQPGGSLRLSCAASGFTFSSYDMSWVRQAPGKCLEWV |
| | | SWISYSGGSIYYADSVKGRFTISRDNSKNTLYLQMNSLRAEDTAVYYCARD |
| | | AQRNSMREFDYWGQGTLVTVSS (SEQ ID NO. 266) |
| | Light | DIQMTQSPSTLSASVGDRVIITCRASRGISSWLAWYQQKPGKAPNLLISKAS |
| | chain of | SLESGVPSRFSGSGSGTDFTLTISSLQPEDFATYYCQQSSSIPLTFGGGTKVEI |
| | B12 | KRTVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTKSF |
| | | NRGEC (SEQ ID NO. 267) |
| | Linker | GSGSGSGSGSGSGSGSGSGGGGSGGGGSGGGGSGGGGSGGGGSGGGGSGS |
| | | GSGSGSGSGSGSGS (SEQ ID NO. 268) |
| | Heavy | QVQLLESGGGLVQPGGSLRLSCAASGFTFSSYWMSWVRQAPGKGLEWVA |
| | chain of | NIKQDGSEKYYVDSVKGRFTISRDNAKNSLYLQMNSLRAEDTAVYYCARV |
| | B12 | ALWDDAFDIWGQGTMVTVSSASTKGPSVFPLAPSSKSTSGGTAALGCLVKD |
| | | YFPEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTLMI |
| | | SRTPEVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVYTLPP |
| | | SREEMTKNQVSLSCAVKGFYPSDIAVEWESNGQPENNYKTTPPVLDSDGSF |
| | | FLVSKLTVDKSRWQQGNVFSCSVMHEALHNHYTQKSLSLSPGK (SEQ ID |
| | | NO. 269) |
| | Linker | GGGGSGGGGSGGGGS (SEQ ID NO. 270) |
| | scFv of | QSVLTQPPSASGTPGQRVTISCSGSSSNIGNNYVTWYQQLPGTAPKLLIYADS |
| | 1A10 | HRPSGVPDRFSGSKSGTSASLAISGLRSEDEADYYCATWDYSLSGYVFGCG |
| | | TKLTVLGGGGSGGGGSGGGGSGGGGSEVQLLESGGGLVQPGGSLRLSCAA |
| | | SGFTFSSYDMSWVRQAPGKCLEWVSWISYSGGSIYYADSVKGRFTISRDNS |
| | | KNTLYLQMNSLRAEDTAVYYCARDAQRNSMREFDYWGQGTLVTVSS (SEQ |
| | | ID NO. 271) |
| | | SEQ ID No. of Nucleotide sequence |
| (HC+LC) | Whole | SEQ ID NO. 303 |
| of C4I | Seq: | |
| + | HC of | |
| scFv of | C4I + | |
| 1A10 | scFv of | |
| | 1A10 | |
| | Heavy | SEQ ID NO. 272 |
| | chain of | |
| | C4I | |
| | Linker | SEQ ID NO. 273 |
| | scFv of | SEQ ID NO. 274 |
| | 1A10 | |
| | Light | SEQ ID NO. 275 |
| | chain of | |
| | C4I | |
| scFab-Fc | Whole | SEQ ID NO. 276 |
| + | Seq | |
| scFv of | Light | SEQ ID NO. 277 |
| 1A10 | chain of | |
| of B12 | B12 | |
| | Linker | SEQ ID NO. 278 |
| | Heavy | SEQ ID NO. 279 |
| | chain of | |
| | B12 | |
| | Linker | SEQ ID NO. 280 |
| | scFv of | SEQ ID NO. 281 |
| | 1A10 | |

### Example 6. In Vitro Activity of the Trispecific Antibodies

To evaluate the ability of trispecific antibodies to promote 4-1BB signal, cell-based 4-1BB assay was performed. In this assay, GloResponse™ NFκB-luc2/4-1BB Jurkat cell line (Promega, cat #CS196004) was used as effector cells and PD-L1 and B7-H3-expressing cancer cell line was used as target cells. GloResponse™ NFκB-luc2/4-1BB Jurkat cell line was genetically modified to stably express 4-1BB and luciferase downstream of a response element. Luciferase expression is induced upon antibody binding to the 4-1BB receptor. The experiment method was performed in accordance with the protocol of the manufacturer. The results are described in FIG. 23.

As shown in FIG. 23, C4I×B6×1A10 and C4I×B12×1A10 trispecific antibodies showed superior 4-1BB signal activation.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An anti-PD-L1/anti-B7-H3 multispecific antibody, comprising an anti-PD-L1 antibody or an antigen-binding fragment thereof and an anti-B7-H3 antibody or an antigen-binding fragment thereof,
wherein the anti-PD-L1 antibody or fragment thereof comprises (1) a VH CDR1 having an amino acid sequence selected from the group consisting of SEQ ID NOs: 1 and 294; (2) a VH CDR2 having an amino acid sequence selected from the group consisting of SEQ ID NOs: 2, 3 and 295; (3) a VH CDR3 having an amino acid sequence selected from the group consisting of SEQ ID NOs: 4, 5, 6, 7, 8, 9, 10, 11 and 296; (4) a VL CDR1 having an amino acid sequence selected from the group consisting of SEQ ID NOs: 12, 13, 14 and 297; (5) a VL CDR2 having an amino acid sequence selected from the group consisting of SEQ ID NOs: 15 and 298; and (6) a VL CDR3 having an amino acid sequence selected from the group consisting of SEQ ID NOs: 16, 17, 18, 19 and 299; and
the anti-B7-H3 antibody or fragment thereof comprises (1) a VH CDR1 having an amino acid sequence selected from the group consisting of SEQ ID NOs: 20, 21, 22 and 23; (2) a VH CDR2 having an amino acid sequence selected from the group consisting of SEQ ID NOs: 24, 25, 26, 27, 28 and 29; and (3) VH CDR3 having an amino acid sequence selected from the group consisting of SEQ ID NOs: 30, 31, 32, 33 and 34; (4) a VL CDR1 having an amino acid sequence selected from the group consisting of SEQ ID NOs: 35, 36, 37, 38 and 39; (5) a VL CDR2 having an amino acid sequence selected from the group consisting of SEQ ID NOs: 40, 41, 42, 43, 44 and 45; and (6) a VL CDR3 having an amino acid sequence selected from the group consisting of SEQ ID NOs:46, 47, 48, 49, and 50.

2. (canceled)

3. The anti-PD-L1/anti-B7-H3 multispecific antibody of claim 1,
wherein the anti-PD-L1 antibody or fragment thereof comprises a light chain variable region having an amino acid sequence selected from the group consisting of SEQ ID NOs: 122, 124, 126, 128, 130, 132, 134, 136, 138, 140 and 209; or a peptide having at least 90% sequence identity to an amino acid sequence selected from the group consisting of SEQ ID NOs: 122, 124, 126, 128, 130, 132, 134, 136, 138, 140 and 209.

4. The anti-PD-L1/anti-B7-H3 multispecific antibody of claim 1,
wherein the anti-B7-H3 antibody or fragment thereof comprises a light chain variable region having an amino acid sequence selected from the group consisting of SEQ ID NOs: 57, 58, 59, 60, 61 and 62; or a peptide having at least 90% sequence identity to an amino acid sequence selected from the group consisting of SEQ ID NOs: 57, 58, 59, 60, 61 and 62.

5. The anti-PD-L1/anti-B7-H3 multispecific antibody of claim 1,
wherein the anti-PD-L1 antibody or fragment thereof comprises a heavy chain variable region having an amino acid sequence selected from the group consisting of SEQ ID NOs: 121, 123, 125, 127, 129, 131, 133, 135, 137, 139 and 211; or a peptide having at least 90% sequence identity to an amino acid sequence selected from the group consisting of SEQ ID NOs: 121, 123, 125, 127, 129, 131, 133, 135, 137, 139 and 211.

6. The anti-PD-L1/anti-B7-H3 multispecific antibody of claim 1,
wherein the anti-B7-H3 antibody or fragment thereof comprises a heavy chain variable region having an amino acid sequence selected from the group consisting of SEQ ID NOs: 51, 52, 53, 54, 55 and 56; or a peptide having at least 90% sequence identity to an amino acid sequence selected from the group consisting of SEQ ID NOs: 51, 52, 53, 54, 55 and 56.

7. The anti-PD-L1/anti-B7-H3 multispecific antibody of claim 1,
wherein the anti-PD-L1 antibody or antigen-binding fragment thereof is capable of binding to at least one of amino acid residues selected from Y134, K162 and N183 of the PD-L1 protein.

8. (canceled)

9. The anti-PD-L1/anti-B7-H3 multispecific antibody of claim 1,
wherein each of the anti-PD-L1 antibody or antigen-binding fragment thereof and the anti-B7-H3 antibody or antigen-binding fragment thereof is independently a chimeric antibody, a humanized antibody, or a fully human antibody.

10. The anti-PD-L1/anti-B7-H3 multispecific antibody of claim 1,
wherein each of the anti-PD-L1 antibody or antigen-binding fragment thereof and the anti-B7-H3 antibody or antigen-binding fragment thereof is independently selected from a group consisting of a whole IgG, Fab, Fab′, F(ab′)2, scFab, dsFv, Fv, scFv, scFv-Fc, scFab-Fc, diabody, minibody, scAb, dAb, half-IgG and combinations thereof.

11-12. (canceled)

13. The anti-PD-L1/anti-B7-H3 multispecific antibody of claim 1, further comprising an anti-4-1BB antibody or an antigen-binding fragment thereof.

14. The anti-PD-L1/anti-B7-H3 multispecific antibody of claim 13,
wherein the anti-4-1BB antibody or an antigen-binding fragment thereof is selected from a group consisting of a whole IgG, Fab, Fab′, F(ab′)2, scFab, dsFv, Fv, scFv, scFv-Fc, scFab-Fc, diabody, minibody, scAb, dAb, half-IgG and combinations thereof.

15. An anti-PD-L1/anti-B7-H3 bispecific antibody, comprising an anti-PD-L1 unit having binding specificity to a human PD-L1 protein and an anti-B7-H3 unit having binding specificity to a human B7-H3 protein.

16. (canceled)

17. The anti-PD-L1/anti-B7-H3 bispecific antibody of claim 15, wherein the anti-PD-L1 unit comprises a PD-L1 binding site located at the N-terminal side of a Fc fragment, and the anti-B7-H3 unit comprises a B7-H3 binding site located at the N-terminal side of the Fc fragment.

18. The anti-PD-L1/anti-B7-H3 bispecific antibody of claim 17, wherein the PD-L1 binding site and the B7-H3 binding site each is independently selected from the group consisting of a Fab fragment, a single chain Fab fragment (scFab), a single-domain antibody (sdAb), scFv, and binding moiety.

19-21. (canceled)

22. The anti-PD-L1/anti-B7-H3 bispecific antibody of claim 15, wherein the anti-PD-L1 binding unit can specifically bind to an immunoglobulin C (Ig C) domain of the human PD-L1 protein, wherein the Ig C domain consists of amino acid residues 133-225.

23. The anti-PD-L1/anti-B7-H3 bispecific antibody of claim 22, wherein the anti-PD-L1 binding unit can specifically bind to amino acid residues Y134, K162, and N183 of the human PD-L1 protein.

24. A pharmaceutical composition comprising the anti-PD-L1/anti-B7-H3 multispecific antibody of claim 1 and a pharmaceutically acceptable carrier.

25. The pharmaceutical composition of claim 24 for treating or preventing a disease associated with PD-L1, B7-H3, or both thereof.

26. The pharmaceutical composition of claim 25, wherein the disease associated with PD-L1, B7-H3, or both thereof is a cancer.

27. A method for treating cancer in a patient in need thereof, comprising administering to the patient an effective amount of the anti-PD-L1/anti-B7-H3 multispecific antibody according to claim 1.

28. The method of claim 27, wherein the cancer is selected from the group consisting of breast cancer, renal cancer, ovarian cancer, gastric cancer, liver cancer, lung cancer, colorectal cancer, pancreatic cancer, skin cancer, bladder cancer, testicular cancer, uterine cancer, prostate cancer, non-small cell lung cancer (NSCLC), neuroblastoma, brain cancer, colon cancer, squamous cell carcinoma, melanoma, myeloma, cervical cancer, thyroid cancer, head and neck cancer and adrenal cancer.

29-30. (canceled)


SEQUENCE LISTING (303 sequences)

SEQ ID NO: 1 - protein, 5 aa, Artificial Sequence
  VH CDR1 of anti-PD-L1 antibody
SYDMS

SEQ ID NO: 2 - protein, 17 aa, Artificial Sequence
  VH CDR2 of anti-PD-L1 antibody
TISDAGGYIYYSDSVKG

SEQ ID NO: 3 - protein, 17 aa, Artificial Sequence
  VH CDR2 of anti-PD-L1 antibody
TISDAGGYIYYRDSVKG

SEQ ID NO: 4 - protein, 10 aa, Artificial Sequence
  VH CDR3 of anti-PD-L1 antibody
EFGKRYALDY

SEQ ID NO: 5 - protein, 10 aa, Artificial Sequence
  VH CDR3 of anti-PD-L1 antibody
ELPWRYALDY

SEQ ID NO: 6 - protein, 10 aa, Artificial Sequence
  VH CDR3 of anti-PD-L1 antibody
EFGKRYALDS

SEQ ID NO: 7 - protein, 10 aa, Artificial Sequence
  VH CDR3 of anti-PD-L1 antibody
EIFNRYALDY

SEQ ID NO: 8 - protein, 10 aa, Artificial Sequence
  VH CDR3 of anti-PD-L1 antibody
ELHFRYALDY

SEQ ID NO: 9 - protein, 10 aa, Artificial Sequence
  VH CDR3 of anti-PD-L1 antibody
ELYFRYALDY

SEQ ID NO: 10 - protein, 10 aa, Artificial Sequence
  VH CDR3 of anti-PD-L1 antibody
ELLHRYALDY

SEQ ID NO: 11 - protein, 10 aa, Artificial Sequence
  VH CDR3 of anti-PD-L1 antibody
ELRGRYALDY

SEQ ID NO: 12 - protein, 11 aa, Artificial Sequence
  VL CDR1 of anti-PD-L1 antibody
KASQDVTPAVA

SEQ ID NO: 13 - protein, 11 aa, Artificial Sequence
  VL CDR1 of anti-PD-L1 antibody
KAKQDVTPAVA

SEQ ID NO: 14 - protein, 11 aa, Artificial Sequence
  VL CDR1 of anti-PD-L1 antibody
KASQDVWPAVA

SEQ ID NO: 15 - protein, 7 aa, Artificial Sequence
  VL CDR2 of anti-PD-L1 antibody
STSSRYT

SEQ ID NO: 16 - protein, 9 aa, Artificial Sequence
  VL CDR3 of anti-PD-L1 antibody
QQHYTTPLT

SEQ ID NO: 17 - protein, 9 aa, Artificial Sequence
  VL CDR3 of anti-PD-L1 antibody
MQHYTTPLT

SEQ ID NO: 18 - protein, 9 aa, Artificial Sequence
  VL CDR3 of anti-PD-L1 antibody
QQHSTTPLT

SEQ ID NO: 19 - protein, 9 aa, Artificial Sequence
  VL CDR3 of anti-PD-L1 antibody
QQHSDAPLT

SEQ ID NO: 20 - protein, 5 aa, Artificial Sequence
  VH CDR1 of anti-B7-H3
DYAMS

SEQ ID NO: 21 - protein, 5 aa, Artificial Sequence
  VH CDR1 of anti-B7-H3
GYYMS

SEQ ID NO: 22 - protein, 5 aa, Artificial Sequence
  VH CDR1 of anti-B7-H3
SYSMS

SEQ ID NO: 23 - protein, 5 aa, Artificial Sequence
  VH CDR1 of anti-B7-H3
SYGMS

SEQ ID NO: 24 - protein, 17 aa, Artificial Sequence
  VH CDR2 of anti-B7-H3
SISSGSGSIYYADSVKG

SEQ ID NO: 25 - protein, 17 aa, Artificial Sequence
  VH CDR2 of anti-B7-H3
LISPSSGSIYYADSVKG

SEQ ID NO: 26 - protein, 17 aa, Artificial Sequence
  VH CDR2 of anti-B7-H3
GIYSDGSNTYYADSVKG

SEQ ID NO: 27 - protein, 17 aa, Artificial Sequence
  VH CDR2 of anti-B7-H3
GISPGGSNTYYADSVKG

SEQ ID NO: 28 - protein, 17 aa, Artificial Sequence
  VH CDR2 of anti-B7-H3
GIYSGGSSKYYADSVKG

SEQ ID NO: 29 - protein, 17 aa, Artificial Sequence
  VH CDR2 of anti-B7-H3
GIYSDASNTYYADSVKG

SEQ ID NO: 30 - protein, 7 aa, Artificial Sequence
  VH CDR3 of anti-B7-H3
NLIPLDY

SEQ ID NO: 31 - protein, 7 aa, Artificial Sequence
  VH CDR3 of anti-B7-H3
GLTKFDY

SEQ ID NO: 32 - protein, 7 aa, Artificial Sequence
  VH CDR3 of anti-B7-H3
MLHRFDY

SEQ ID NO: 33 - protein, 12 aa, Artificial Sequence
  VH CDR3 of anti-B7-H3
DAWIARLLLFDY

SEQ ID NO: 34 - protein, 7 aa, Artificial Sequence
  VH CDR3 of anti-B7-H3
NRLRFDY

SEQ ID NO: 35 - protein, 13 aa, Artificial Sequence
  VL CDR1 of anti-B7-H3
SGSSSNIGSNAVS

SEQ ID NO: 36 - protein, 13 aa, Artificial Sequence
  VL CDR1 of anti-B7-H3
TGSSSNIGSNDVS

SEQ ID NO: 37 - protein, 13 aa, Artificial Sequence
  VL CDR1 of anti-B7-H3
SGSSSNIGSNSVT

SEQ ID NO: 38 - protein, 13 aa, Artificial Sequence
  VL CDR1 of anti-B7-H3
SGSSSNIGSNAVT

SEQ ID NO: 39 - protein, 13 aa, Artificial Sequence
  VL CDR1 of anti-B7-H3
TGSSSNIGSNSVT

SEQ ID NO: 40 - protein, 7 aa, Artificial Sequence
  VL CDR2 of anti-B7-H3
YNSHRPS

SEQ ID NO: 41 - protein, 7 aa, Artificial Sequence
  VL CDR2 of anti-B7-H3
ANSHRPS

SEQ ID NO: 42 - protein, 7 aa, Artificial Sequence
  VL CDR2 of anti-B7-H3
ADSQRPS

SEQ ID NO: 43 - protein, 7 aa, Artificial Sequence
  VL CDR2 of anti-B7-H3
YNNKRPS

SEQ ID NO: 44 - protein, 7 aa, Artificial Sequence
  VL CDR2 of anti-B7-H3
SDSHRPS

SEQ ID NO: 45 - protein, 7 aa, Artificial Sequence
  VL CDR2 of anti-B7-H3
ADVQRPS

SEQ ID NO: 46 - protein, 11 aa, Artificial Sequence
  VL CDR3 of anti-B7-H3
GSWDASLNAYV

SEQ ID NO: 47 - protein, 11 aa, Artificial Sequence
  VL CDR3 of anti-B7-H3
GSWDDSLSGYV

SEQ ID NO: 48 - protein, 11 aa, Artificial Sequence
  VL CDR3 of anti-B7-H3
GTWDSSLNAYV

SEQ ID NO: 49 - protein, 11 aa, Artificial Sequence
  VL CDR3 of anti-B7-H3
GTWDDSLSGYV

SEQ ID NO: 50 - protein, 11 aa, Artificial Sequence
  VL CDR3 of anti-B7-H3
GTWDASLNAYV

SEQ ID NO: 51 - protein, 116 aa, Artificial Sequence
  heavy chain variable region of anti-B7-H3
EVQLLESGGGLVQPGGSLRLSCAASGFTFSDYAMSWVRQAPGKGLEWVSSISSGSGSIYYADSVKGRFTISRDNSKNTLYLQMNSLRAEDTAVYYCAKNLIPLDYWGQGTLVTVSS

SEQ ID NO: 52 - protein, 116 aa, Artificial Sequence
  heavy chain variable region of anti-B7-H3
EVQLLESGGGLVQPGGSLRLSCAASGFTFSGYYMSWVRQAPGKGLEWVSLISPSSGSIYYADSVKGRFTISRDNSKNTLYLQMNSLRAEDTAVYYCAKGLTKFDYWGQGTLVTVSS

SEQ ID NO: 53 - protein, 116 aa, Artificial Sequence
  heavy chain variable region of anti-B7-H3
EVQLLESGGGLVQPGGSLRLSCAASGFTFSSYSMSWVRQAPGKGLEWVSGIYSDGSNTYYADSVKGRFTISRDNSKNTLYLQMNSLRAEDTAVYYCAKMLHRFDYWGQGTLVTVSS

SEQ ID NO: 54 - protein, 121 aa, Artificial Sequence
  heavy chain variable region of anti-B7-H3
EVQLLESGGGLVQPGGSLRLSCAASGFTFSDYAMSWVRQAPGKGLEWVSGISPGGSNTYYADSVKGRFTISRDNSKNTLYLQMNSLRAEDTAVYYCAKDAWIARLLLFDYWGQGTLVTVSS

SEQ ID NO: 55 - protein, 116 aa, Artificial Sequence
  heavy chain variable region of anti-B7-H3
EVQLLESGGGLVQPGGSLRLSCAASGFTFSSYGMSWVRQAPGKGLEWVSGIYSGGSSKYYADSVKGRFTISRDNSKNTLYLQMNSLRAEDTAVYYCAKNRLRFDYWGQGTLVTVSS

SEQ ID NO: 56 - protein, 116 aa, Artificial Sequence
  heavy chain variable region of anti-B7-H3
EVQLLESGGGLVQPGGSLRLSCAASGFTFSSYSMSWVRQAPGKGLEWVSGIYSDASNTYYADSVKGRFTISRDNSKNTLYLQMNSLRAEDTAVYYCAKMLHRFDYWGQGTLVTVSS

SEQ ID NO: 57 - protein, 110 aa, Artificial Sequence
  light chain variable region of anti-B7-H3
QSVLTQPPSASGTPGQRVTISCSGSSSNIGSNAVSWYQQLPGTAPKLLIYYNSHRPSGVPDRFSGSKSGTSASLAISGLRSEDEADYYCGSWDASLNAYVFGGGTKLTVL

SEQ ID NO: 58 - protein, 110 aa, Artificial Sequence
  light chain variable region of anti-B7-H3
QSVLTQPPSASGTPGQRVTISCTGSSSNIGSNDVSWYQQLPGTAPKLLIYANSHRPSGVPDRFSGSKSGTSASLAISGLRSEDEADYYCGSWDDSLSGYVFGGGTKLTVL

SEQ ID NO: 59 - protein, 110 aa, Artificial Sequence
  light chain variable region of anti-B7-H3
QSVLTQPPSASGTPGQRVTISCSGSSSNIGSNSVTWYQQLPGTAPKLLIYADSQRPSGVPDRFSGSKSGTSASLAISGLRSEDEADYYCGTWDSSLNAYVFGGGTKLTVL

SEQ ID NO: 60 - protein, 110 aa, Artificial Sequence
  light chain variable region of anti-B7-H3
QSVLTQPPSASGTPGQRVTISCSGSSSNIGSNAVTWYQQLPGTAPKLLIYYNNKRPSGVPDRFSGSKSGTSASLAISGLRSEDEADYYCGTWDDSLSGYVFGGGTKLTVL

SEQ ID NO: 61 - protein, 110 aa, Artificial Sequence
  light chain variable region of anti-B7-H3
QSVLTQPPSASGTPGQRVTISCTGSSSNIGSNSVTWYQQLPGTAPKLLIYSDSHRPSGVPDRFSGSKSGTSASLAISGLRSEDEADYYCGTWDASLNAYVFGGGTKLTVL

SEQ ID NO: 62 - protein, 110 aa, Artificial Sequence
  light chain variable region of anti-B7-H3
QSVLTQPPSASGTPGQRVTISCSGSSSNIGSNSVTWYQQLPGTAPKLLIYADVQRPSGVPDRFSGSKSGTSASLAISGLRSEDEADYYCGTWDSSLNAYVFGGGTKLTVL

SEQ ID NO: 63 - protein, 119 aa, Artificial Sequence
  HL1210-3 VH
EVKLVESGGDLVKPGGSLKLSCAASGFTFSSYDMSWVRQTPEKSLEWVATISDGGGYIYYSDSVKGRFTISRDNAKNNLYLQMSSLRSEDTALYICAREFGKRYALDYWGQGTSVTVSS

SEQ ID NO: 64 - protein, 107 aa, Artificial Sequence
  HL1210-3 VL
DIVMTQSHKFMSTSVGDRVSISCKASQDVTPAVAWYQQKPGQSPKLLIYSTSSRYTGVPDRFTGSGSGTDFTFTISSVQAEDLAVYYCQQHYTTPLTFGAGTKLELK

SEQ ID NO: 65 - DNA, 357 bp, Artificial Sequence
  HL1210-3 VH
gaagtgaaactggtggagtctgggggagacttagtgaagcctggagggtccctgaaactctcctgtgcagcctctggattcactttcagtagctatgacatgtcttgggttcgccagactccggagaagagtctggagtgggtcgcaaccattagtgatggtggtggttacatctactattcagacagtgtgaaggggcgatttaccatctccagagacaatgccaagaacaacctgtacctgcaaatgagcagtctgaggtctgaggacacggccttgtatatttgtgcaagagaatttggtaagcgctatgctttggactactggggtcaaggaacctcagtcaccgtctcctca

SEQ ID NO: 66 - DNA, 321 bp, Artificial Sequence
  HL1210-3 VL
gacattgtgatgacccagtctcacaaattcatgtccacatcggtaggagacagggtcagcatctcctgcaaggccagtcaggatgtgactcctgctgtcgcctggtatcaacagaagccaggacaatctcctaaactactgatttactccacatcctcccggtacactggagtccctgatcgcttcactggcagtggatctgggacggatttcactttcaccatcagcagtgtgcaggctgaagacctggcagtttattactgtcagcaacattatactactccgctcacgttcggtgctgggaccaagctggagctgaaa

SEQ ID NO: 67 - protein, 119 aa, Artificial Sequence; identical to SEQ ID NO: 63
  HL 1210-VH

SEQ ID NO: 68 - protein, 119 aa, Artificial Sequence
  Hu1210 VH.1
EVQLVESGGGLVKPGGSLRLSCAASGFTFSSYDMSWVRQAPGKGLEWVSTISDGGGYIYYSDSVKGRFTISRDNAKNSLYLQMNSLRAEDTAVYYCAREFGKRYALDYWGQGTTVTVSS

SEQ ID NO: 69 - protein, 119 aa, Artificial Sequence
  Hu1210 VH.1a
EVQLVESGGGLVKPGGSLRLSCAASGFTFSSYDMSWVRQAPGKGLEWVATISDGGGYIYYSDSVKGRFTISRDNAKNSLYLQMNSLRAEDTAVYYCAREFGKRYALDYWGQGTTVTVSS

SEQ ID NO: 70 - protein, 119 aa, Artificial Sequence
  Hu1210 VH.1b
EVQLVESGGGLVKPGGSLRLSCAASGFTFSSYDMSWVRQAPGKSLEWVATISDGGGYIYYSDSVKGRFTISRDNAKNSLYLQMNSLRAEDTAVYICAREFGKRYALDYWGQGTTVTVSS

SEQ ID NO: 71 - protein, 119 aa, Artificial Sequence
  Hu1210 VH.2
EVQLVESGGGLVKPGGSLRLSCAASGFTFSSYDMSWIRQAPGKGLEWVSTISDGGGYIYYSDSVKGRFTISRDNAKNSLYLQMNSLRAEDTAVYYCAREFGKRYALDYWGQGTTVTVSS

SEQ ID NO: 72 - protein, 119 aa, Artificial Sequence
  Hu1210 VH.2a
EVQLVESGGGLVKPGGSLRLSCAASGFTFSSYDMSWIRQAPGKGLEWVATISDGGGYIYYSDSVKGRFTISRDNAKNSLYLQMNSLRAEDTAVYYCAREFGKRYALDYWGQGTTVTVSS

SEQ ID NO: 73 - protein, 119 aa, Artificial Sequence; identical to SEQ ID NO: 70
  Hu1210 VH.2b

SEQ ID NO: 74 - protein, 119 aa, Artificial Sequence
  Hu1210 VH.3
EVQLLESGGGLVQPGGSLRLSCAASGFTFSSYDMSWVRQAPGKGLEWVSTISDGGGYIYYSDSVKGRFTISRDNSKNTLYLQMNSLRAEDTAVYYCAREFGKRYALDYWGQGTTVTVSS

SEQ ID NO: 75 - protein, 119 aa, Artificial Sequence
  Hu1210 VH.3a
EVQLLESGGGLVQPGGSLRLSCAASGFTFSSYDMSWVRQAPGKSLEWVATISDGGGYIYYSDSVKGRFTISRDNSKNTLYLQMNSLRAEDTAVYICAREFGKRYALDYWGQGTTVTVSS

SEQ ID NO: 76 - protein, 119 aa, Artificial Sequence
  Hu1210 VH.4
EVQLVESGGGLVQPGGSLRLSCAASGFTFSSYDMSWVRQAPGKGLEWVSTISDGGGYIYYSDSVKGRFTISRDNAKNSLYLQMNSLRDEDTAVYYCAREFGKRYALDYWGQGTTVTVSS

SEQ ID NO: 77 - protein, 119 aa, Artificial Sequence
  Hu1210 VH.4a
EVQLVESGGGLVQPGGSLRLSCAASGFTFSSYDMSWVRQAPGKGLEWVATISDGGGYIYYSDSVKGRFTISRDNAKNSLYLQMNSLRDEDTAVYYCAREFGKRYALDYWGQGTTVTVSS

SEQ ID NO: 78 - protein, 119 aa, Artificial Sequence
  Hu1210 VH.4b
EVQLVESGGGLVQPGGSLRLSCAASGFTFSSYDMSWVRQAPGKSLEWVATISDGGGYIYYSDSVKGRFTISRDNAKNSLYLQMNSLRDEDTAVYICAREFGKRYALDYWGQGTTVTVSS

SEQ ID NO: 79 - protein, 119 aa, Artificial Sequence
  Hu1210 VH.4c
EVQLVESGGGLVQPGGSLRLSCAASGFTFSSYDMSWVRQAPGKSLEWVATISEGGGYIYYSDSVKGRFTISRDNAKNSLYLQMNSLRDEDTAVYICAREFGKRYALDYWGQGTTVTVSS

SEQ ID NO: 80 - protein, 119 aa, Artificial Sequence
  Hu1210 VH.4d (H12 VH)
EVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSS

SEQ ID NO: 81 - protein, 119 aa, Artificial Sequence
  Hu1210 VH.4e
EVQLVESGGGLVQPGGSLRLSCAASGFTFSSYDMSWVRQAPGKSLEWVATISDVGGYIYYSDSVKGRFTISRDNAKNSLYLQMNSLRDEDTAVYICAREFGKRYALDYWGQGTTVTVSS

SEQ ID NO: 82 - protein, 119 aa, Artificial Sequence
  Hu1210 VH.5
EVQLVESGGGLVQPGGSLRLSCAASGFTFSSYDMSWVRQAPGKGLEWVATISDGGGYIYYSDSVKGRFTISRDNAKNSLYLQMNSLRAEDTAVYYCAREFGKRYALDYWGQGTLVTVSS

SEQ ID NO: 83 - protein, 119 aa, Artificial Sequence
  HU1210 VH.5a
EVQLVESGGGLVQPGGSLRLSCAASGFTFSSYDMSWVRQAPGKGLEWVATISDGGGYIYYSDSVKGRFTISRDNAKNSLYLQMNSLRAEDTAVYICAREFGKRYALDYWGQGTLVTVSS

SEQ ID NO: 84 - protein, 119 aa, Artificial Sequence
  HU1210 VH.5b
EVQLVESGGGLVQPGGSLRLSCAASGFTFSSYDMSWVRQAPGKGLEWVATISDGGGYIYYSDSVKGRFTISRDNAKNSLYLQMNSLRAEDTAVYICAREFGKRYALDYWGQGTTVTVSS

SEQ ID NO: 85 - protein, 119 aa, Artificial Sequence
  HU1210 VH.5C
EVQLVESGGGLVQPGGSLRLSCAASGFTFSSYDMSWVRQAPGKGLEWVATISDGGGYIYYSDSVKGRFTISRDNAKNNLYLQMNSLRAEDTAVYICAREFGKRYALDYWGQGTLVTVSS

SEQ ID NO: 86 - protein, 119 aa, Artificial Sequence
  HU1210 VH.5d
EVQLVESGGGLVQPGGSLRLSCAASGFTFSSYDMSWVRQTPEKSLEWVATISDGGGYIYYSDSVKGRFTISRDNAKNNLYLQMNSLRAEDTAVYICAREFGKRYALDYWGQGTLVTVSS

SEQ ID NO: 87 - protein, 107 aa, Artificial Sequence; identical to SEQ ID NO: 64
  HL1210-VK

SEQ ID NO: 88 - protein, 107 aa, Artificial Sequence
  Hu1210 VK.1 (H12 VL)
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIK

SEQ ID NO: 89 - protein, 107 aa, Artificial Sequence
  Hu1210 VL.1a
DIQMTQSPSSLSASVGDRVTITCKASQDVTPAVAWYQQKPGKSPKLLIYSTSSRYTGVPSRFSGSGSGTDFTFTISSLQPEDIATYYCQQHYTTPLTFGQGTKLEIK

SEQ ID NO: 90 - protein, 108 aa, Artificial Sequence
  Hu1210 VK.2
DIQMTQSPSSLSASVGDRVTITCKASQDVTPAVAWYQQKPGKAPKLLIYSTSSRYTGVPSRFSGSGSGTDFTLTISSLQPEDFATYYCQQHYTTPLTFGQGTKLEIKR

SEQ ID NO: 91 - protein, 108 aa, Artificial Sequence
  Hu1210 VK.2a
DIQMTQSPSSLSASVGDRVTITCKASQDVTPAVAWYQQKPGKAPKLLIYSTSSRYTGVPDRFTGSGSGTDFTLTISSLQPEDFATYYCQQHYTTPLTFGQGTKLEIKR

SEQ ID NO: 92 - protein, 108 aa, Artificial Sequence
  Hu1210 VK.2b
DIQMTQSPSSLSASVGDRVTITCKASQDVTPAVAWYQQKPGQSPKLLIYSTSSRYTGVPDRFTGSGSGTDFTLTISSLQPEDFATYYCQQHYTTPLTFGQGTKLEIKR

SEQ ID NO: 93 - protein, 108 aa, Artificial Sequence
  Hu1210 VK.2c
DIQMTQSPSSLSASVGDRVTISCKASQDVTPAVAWYQQKPGQSPKLLIYSTSSRYTGVPDRFTGSGSGTDFTLTISSLQPEDFATYYCQQHYTTPLTFGQGTKLEIKR

SEQ ID NO: 94 - DNA, 357 bp, Artificial Sequence
  HL1210-VH
gaggtgaagctggtggagagcggcggagatctggtgaagcctggcggcagcctgaagctgagctgtgccgccagcggcttcaccttcagcagctacgacatgagctgggtgaggcagacccccgagaagagcctggagtgggtggccaccatcagcgatggcggcggctacatctactacagcgacagcgtgaagggcaggttcaccatcagcagggacaacgccaagaacaacctgtacctgcagatgagcagcctgaggagcgaggacaccgccctgtacatctgcgccagggagttcggcaagaggtacgccctggactactggggacagggcaccagcgtgaccgtgagcagc

SEQ ID NO: 95 - DNA, 357 bp, Artificial Sequence
  Hu1210 VH.1
gaggtgcagctggtggagagcggaggaggactggtgaagcccggaggcagcctgagactgagctgcgctgccagcggcttcaccttcagcagctacgacatgagctgggtgagacaggcccctggcaaaggcctggagtgggtgagcaccatctccgatggcggcggctacatctattactccgacagcgtgaagggcaggttcaccatcagcagggacaacgccaagaacagcctgtacctgcagatgaacagcctgagggccgaggacaccgccgtgtactactgcgccagggagttcggcaaaaggtacgccctggactactggggccagggcacaaccgtgaccgtgagcagc

SEQ ID NO: 96 - DNA, 357 bp, Artificial Sequence
  Hu1210 VH.1a
gaggtgcagctggtggagagcggaggaggactggtgaagcccggaggcagcctgagactgagctgcgctgccagcggcttcaccttcagcagctacgacatgagctgggtgagacaggcccctggcaaaggcctggagtgggtggccaccatctccgatggcggcggctacatctattactccgacagcgtgaagggcaggttcaccatcagcagggacaacgccaagaacagcctgtacctgcagatgaacagcctgagggccgaggacaccgccgtgtactactgcgccagggagttcggcaaaaggtacgccctggactactggggccagggcacaaccgtgaccgtgagcagc

SEQ ID NO: 97 - DNA, 357 bp, Artificial Sequence
  Hu1210 VH.1b
gaggtgcagctggtggagagcggaggaggactggtgaagcccggaggcagcctgagactgagctgcgctgccagcggcttcaccttcagcagctacgacatgagctgggtgagacaggcccctggcaaaagcctggagtgggtggccaccatctccgatggcggcggctacatctattactccgacagcgtgaagggcaggttcaccatcagcagggacaacgccaagaacagcctgtacctgcagatgaacagcctgagggccgaggacaccgccgtgtacatctgcgccagggagttcggcaaaaggtacgccctggactactggggccagggcacaaccgtgaccgtgagcagc

SEQ ID NO: 98 - DNA, 357 bp, Artificial Sequence
  Hu1210 VH.2
gaggtgcagctggtggagagcggaggaggactggtgaagcccggaggcagcctgagactgagctgcgctgccagcggcttcaccttcagcagctacgacatgagctggatcagacaggcccctggcaaaggcctggagtgggtgagcaccatctccgatggcggcggctacatctattactccgacagcgtgaagggcaggttcaccatcagcagggacaacgccaagaacagcctgtacctgcagatgaacagcctgagggccgaggacaccgccgtgtactactgcgccagggagttcggcaaaaggtacgccctggactactggggccagggcacaaccgtgaccgtgagcagc

SEQ ID NO: 99 - DNA, 357 bp, Artificial Sequence
  Hu1210 VH.2a
gaggtgcagctggtggagagcggaggaggactggtgaagcccggaggcagcctgagactgagctgcgctgccagcggcttcaccttcagcagctacgacatgagctggatcagacaggcccctggcaaaggcctggagtgggtggccaccatctccgatggcggcggctacatctattactccgacagcgtgaagggcaggttcaccatcagcagggacaacgccaagaacagcctgtacctgcagatgaacagcctgagggccgaggacaccgccgtgtactactgcgccagggagttcggcaaaaggtacgccctggactactggggccagggcacaaccgtgaccgtgagcagc

SEQ ID NO: 100 - DNA, 357 bp, Artificial Sequence; identical to SEQ ID NO: 97
  Hu1210 VH.2b

SEQ ID NO: 101 - DNA, 357 bp, Artificial Sequence
  Hu1210 VH.3
gaggtgcagctgctggagagcggaggaggactggtgcaacccggaggcagcctgagactgagctgcgctgccagcggcttcaccttcagcagctacgacatgagctgggtgagacaggcccctggcaaaggcctggagtgggtgagcaccatctccgatggcggcggctacatctattactccgacagcgtgaagggcaggttcaccatcagcagggacaacagcaagaacaccctgtacctgcagatgaacagcctgagggccgaggacaccgccgtgtactactgcgccagggagttcggcaaaaggtacgccctggactactggggccagggcacaaccgtgaccgtgagcagc

SEQ ID NO: 102 - DNA, 357 bp, Artificial Sequence
  Hu1210 VH.3a
gaggtgcagctgctggagagcggaggaggactggtgcaacccggaggcagcctgagactgagctgcgctgccagcggcttcaccttcagcagctacgacatgagctgggtgagacaggcccctggcaaaagcctggagtgggtggccaccatctccgatggcggcggctacatctattactccgacagcgtgaagggcaggttcaccatcagcagggacaacagcaagaacaccctgtacctgcagatgaacagcctgagggccgaggacaccgccgtgtacatctgcgccagggagttcggcaaaaggtacgccctggactactggggccagggcacaaccgtgaccgtgagcagc

SEQ ID NO: 103 - DNA, 357 bp, Artificial Sequence
  Hu1210 VH.4
gaggtgcagctggtggagagcggaggaggactggtgcaacccggaggcagcctgagactgagctgcgctgccagcggcttcaccttcagcagctacgacatgagctgggtgagacaggcccctggcaaaggcctggagtgggtgagcaccatctccgatggcggcggctacatctattactccgacagcgtgaagggcaggttcaccatcagcagggacaacgccaagaacagcctgtacctgcagatgaacagcctgagggatgaggacaccgccgtgtactactgcgccagggagttcggcaaaaggtacgccctggactactggggccagggcacaaccgtgaccgtgagcagc

SEQ ID NO: 104 - DNA, 357 bp, Artificial Sequence
  Hu1210 VH.4a
gaggtgcagctggtggagagcggaggaggactggtgcaacccggaggcagcctgagactgagctgcgctgccagcggcttcaccttcagcagctacgacatgagctgggtgagacaggcccctggcaaaggcctggagtgggtggccaccatctccgatggcggcggctacatctattactccgacagcgtgaagggcaggttcaccatcagcagggacaacgccaagaacagcctgtacctgcagatgaacagcctgagggatgaggacaccgccgtgtactactgcgccagggagttcggcaaaaggtacgccctggactactggggccagggcacaaccgtgaccgtgagcagc

SEQ ID NO: 105 - DNA, 357 bp, Artificial Sequence
  Hu1210 VH.4b
gaggtgcagctggtggagagcggaggaggactggtgcaacccggaggcagcctgagactgagctgcgctgccagcggcttcaccttcagcagctacgacatgagctgggtgagacaggcccctggcaaaagcctggagtgggtggccaccatctccgatggcggcggctacatctattactccgacagcgtgaagggcaggttcaccatcagcagggacaacgccaagaacagcctgtacctgcagatgaacagcctgagggatgaggacaccgccgtgtacatctgcgccagggagttcggcaaaaggtacgccctggactactggggccagggcacaaccgtgaccgtgagcagc

SEQ ID NO: 106 - DNA, 357 bp, Artificial Sequence
  Hu1210 VH.4c
gaggtgcagctggtggagagcggaggaggactggtgcaacccggaggcagcctgagactgagctgcgctgccagcggcttcaccttcagcagctacgacatgagctgggtgagacaggcccctggcaaaagcctggagtgggtggccaccatctccgaaggcggcggctacatctattactccgacagcgtgaagggcaggttcaccatcagcagggacaacgccaagaacagcctgtacctgcagatgaacagcctgagggatgaggacaccgccgtgtacatctgcgccagggagttcggcaaaaggtacgccctggactactggggccagggcacaaccgtgaccgtgagcagc

SEQ ID NO: 107 - DNA, 357 bp, Artificial Sequence
  Hu1210 VH.4d (H12 VH)
gaggtgcagctggtggagagcggaggaggactggtgcaacccggaggcagcctgagactgagctgcgctgccagcggcttcaccttcagcagctacgacatgagctgggtgagacaggcccctggcaaaagcctggagtgggtggccaccatctccgatgcgggcggctacatctattactccgacagcgtgaagggcaggttcaccatcagcagggacaacgccaagaacagcctgtacctgcagatgaacagcctgagggatgaggacaccgccgtgtacatctgcgccagggagttcggcaaaaggtacgccctggactactggggccagggcacaaccgtgaccgtgagcagc

SEQ ID NO: 108 - DNA, 357 bp, Artificial Sequence
  Hu1210 VH.4e
gaggtgcagctggtggagagcggaggaggactggtgcaacccggaggcagcctgagactgagctgcgctgccagcggcttcaccttcagcagctacgacatgagctgggtgagacaggcccctggcaaaagcctggagtgggtggccaccatctccgatgttggcggctacatctattactccgacagcgtgaagggcaggttcaccatcagcagggacaacgccaagaacagcctgtacctgcagatgaacagcctgagggatgaggacaccgccgtgtacatctgcgccagggagttcggcaaaaggtacgccctggactactggggccagggcacaaccgtgaccgtgagcagc

SEQ ID NO: 109 - DNA, 357 bp, Artificial Sequence
  Hu1210 VH.5
gaggtgcagctggtggagtccggaggaggcctggtgcaacctggaggctccctgaggctgtcctgtgccgcttccggcttcaccttcagctcctacgatatgagctgggtgaggcaggctcctggaaagggcctggagtgggtggccaccatctccgacggaggcggctacatctactactccgactccgtgaagggcaggttcaccatctcccgggacaacgccaagaactccctgtacctgcagatgaactctctcagggctgaggacaccgccgtgtattactgcgccagggagtttggcaagaggtacgccctggattactggggccagggcacactggtgacagtgagctcc

SEQ ID NO: 110 - DNA, 357 bp, Artificial Sequence
  HU1210 VH.5a
gaggtgcagctggtggagtccggaggaggcctggtgcaacctggaggctccctgaggctgtcctgtgccgcttccggcttcaccttcagctcctacgatatgagctgggtgaggcaggctcctggaaagggcctggagtgggtggccaccatctccgacggaggcggctacatctactactccgactccgtgaagggcaggttcaccatctcccgggacaacgccaagaactccctgtacctgcagatgaactctctcagggctgaggacaccgccgtgtatatctgcgccagggagtttggcaagaggtacgccctggattactggggccagggcacactggtgacagtgagctcc

SEQ ID NO: 111 - DNA, 357 bp, Artificial Sequence
  HU1210 VH.5b
gaggtgcagctggtggagtccggaggaggcctggtgcaacctggaggctccctgaggctgtcctgtgccgcttccggcttcaccttcagctcctacgatatgagctgggtgaggcaggctcctggaaagggcctggagtgggtggccaccatctccgacggaggcggctacatctactactccgactccgtgaagggcaggttcaccatctcccgggacaacgccaagaacaacctgtacctgcagatgaactctctcagggctgaggacaccgccgtgtatatctgcgccagggagtttggcaagaggtacgccctggattactggggccagggcacactggtgacagtgagctcc

SEQ ID NO: 112 - DNA, 357 bp, Artificial Sequence
  HU1210 VH.5C
gaggtgcagctggtggagtccggaggaggcctggtgcaacctggaggctccctgaggctgtcctgtgccgcttccggcttcaccttcagctcctacgatatgagctgggtgaggcagacccctgagaagagcctggagtgggtggccaccatctccgacggaggcggctacatctactactccgactccgtgaagggcaggttcaccatctcccgggacaacgccaagaacaacctgtacctgcagatgaactctctcagggctgaggacaccgccgtgtatatctgcgccagggagtttggcaagaggtacgccctggattactggggccagggcacactggtgacagtgagctcc

SEQ ID NO: 113 - DNA, 357 bp, Artificial Sequence
  HU1210 VH.5d
gaggtgcagctggtggagtccggaggaggcctggtgcaacctggaggctccctgaggctgtcctgtgccgcttccggcttcaccttcagctcctacgatatgagctgggtgaggcaggctcctggaaagggcctggagtgggtggccaccatctccgacggaggcggctacatctactactccgactccgtgaagggcaggttcaccatctcccgggacaacgccaagaactccctgtacctgcagatgaactctctcagggctgaggacaccgccgtgtatatctgcgccagggagtttggcaagaggtacgccctggattactggggccagggcacaaccgtgacagtgagctcc

SEQ ID NO: 114 - DNA, 321 bp, Artificial Sequence
  HL1210-VK
gacatcgtgatgacccagagccacaagttcatgagcaccagcgtgggcgatagggtgagcatcagctgcaaggccagccaggatgtgacccctgccgtggcctggtaccagcagaagcccggccagagccccaagctgctgatctacagcaccagcagcaggtacaccggcgtgcccgacaggttcacaggaagcggcagcggcaccgacttcaccttcaccatcagcagcgtgcaggccgaggacctggccgtgtactactgccagcagcactacaccacccctctgaccttcggcgccggcaccaagctggagctgaag

SEQ ID NO: 115 - DNA, 321 bp, Artificial Sequence
  Hu1210 VK.1 (H12 VL)
gacatccagatgacccagagccctagcagcctgagcgctagcgtgggcgacagggtgaccatcacctgcaaggccagccaggatgtgacccctgccgtggcctggtaccagcagaagcccggcaaggcccccaagctgctgatctacagcaccagcagcaggtacaccggcgtgcccagcaggtttagcggaagcggcagcggcaccgacttcaccttcaccatcagcagcctgcagcccgaggacatcgccacctactactgccagcagcactacaccacccctctgaccttcggccagggcaccaagctggagatcaag

SEQ ID NO: 116 - DNA, 321 bp, Artificial Sequence
  Hu1210 VK.1a
gacatccagatgacccagagccctagcagcctgagcgctagcgtgggcgacagggtgaccatcacctgcaaggccagccaggatgtgacccctgccgtggcctggtaccagcagaagcccggcaagtcccccaagctgctgatctacagcaccagcagcaggtacaccggcgtgcccagcaggtttagcggaagcggcagcggcaccgacttcaccttcaccatcagcagcctgcagcccgaggacatcgccacctactactgccagcagcactacaccacccctctgaccttcggccagggcaccaagctggagatcaag

SEQ ID NO: 117 - DNA, 324 bp, Artificial Sequence
  Hu1210 VK.2
gacattcagatgacccagtcccctagcagcctgtccgcttccgtgggcgacagggtgaccatcacctgcaaggccagccaggacgtgacacctgctgtggcctggtatcaacagaagcctggcaaggctcctaagctcctgatctacagcacatcctcccggtacaccggagtgccctccaggtttagcggcagcggctccggcaccgatttcaccctgaccatttcctccctgcagcccgaggacttcgccacctactactgccagcagcactacaccacacccctgaccttcggccagggcaccaagctggagatcaagcgg

SEQ ID NO: 118 - DNA, 324 bp, Artificial Sequence
  Hu1210 VK.2a
gacattcagatgacccagtcccctagcagcctgtccgcttccgtgggcgacagggtgaccatcacctgcaaggccagccaggacgtgacacctgctgtggcctggtatcaacagaagcctggcaaggctcctaagctcctgatctacagcacatcctcccggtacaccggagtgcccgacaggtttaccggcagcggctccggcaccgatttcaccctgaccatttcctccctgcagcccgaggacttcgccacctactactgccagcagcactacaccacacccctgaccttcggccagggcaccaagctggagatcaagcgg

SEQ ID NO: 119 - DNA, 324 bp, Artificial Sequence
  Hu1210 VK.2b
gacattcagatgacccagtcccctagcagcctgtccgcttccgtgggcgacagggtgaccatcacctgcaaggccagccaggacgtgacacctgctgtggcctggtatcaacagaagcctggccagagccctaagctcctgatctacagcacatcctcccggtacaccggagtgcccgacaggtttaccggcagcggctccggcaccgatttcaccctgaccatttcctccctgcagcccgaggacttcgccacctactactgccagcagcactacaccacacccctgaccttcggccagggcaccaagctggagatcaagcgg

SEQ ID NO: 120 - DNA, 324 bp, Artificial Sequence
  Hu1210 VK.2c
gacattcagatgacccagtcccctagcagcctgtccgcttccgtgggcgacagggtgaccatcagctgcaaggccagccaggacgtgacacctgctgtggcctggtatcaacagaagcctggccagagccctaagctcctgatctacagcacatcctcccggtacaccggagtgcccgacaggtttaccggcagcggctccggcaccgatttcaccctgaccatttcctccctgcagcccgaggacttcgccacctactactgccagcagcactacaccacacccctgaccttcggccagggcaccaagctggagatcaagcgg

SEQ ID NO: 121 - protein, 119 aa, Artificial Sequence; identical to SEQ ID NO: 80
  WT-VH

SEQ ID NO: 122 - protein, 107 aa, Artificial Sequence; identical to SEQ ID NO: 88
  WT-VK

SEQ ID NO: 123 - protein, 119 aa, Artificial Sequence
  B3-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICAREFGKRYALDYWGQGTTVTVSS

SEQ ID NO: 124 - protein, 107 aa, Artificial Sequence
  B3-VK
DIQMTQSPSSLSASVGDRVTITCKAKQDVTPAVAWYQQKPGKAPKLLIYSTSSRYTGVPSRFSGSGSGTDFTFTISSLQPEDIATYYCMQHYTTPLTFGQGTKLEIK

SEQ ID NO: 125 - protein, 119 aa, Artificial Sequence
  C4-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICAREFGKRYALDSWGQGTTVTVSS

SEQ ID NO: 126 - protein, 107 aa, Artificial Sequence
  C4-VK
DIQMTQSPSSLSASVGDRVTITCKASQDVWPAVAWYQQKPGKAPKLLIYSTSSRYTGVPSRFSGSGSGTDFTFTISSLQPEDIATYYCQQHSTTPLTFGQGTKLEIK

SEQ ID NO: 127 - protein, 119 aa, Artificial Sequence
  B1-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICAREIFNRYALDYWGQGTTVTVSS

SEQ ID NO: 128 - protein, 107 aa, Artificial Sequence; identical to SEQ ID NO: 88
  B1-VK

SEQ ID NO: 129 - protein, 119 aa, Artificial Sequence
  B6-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSS

SEQ ID NO: 130 - protein, 107 aa, Artificial Sequence; identical to SEQ ID NO: 88
  B6-VK

SEQ ID NO: 131 - protein, 119 aa, Artificial Sequence
  C3-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELHFRYALDYWGQGTTVTVSS

SEQ ID NO: 132 - protein, 107 aa, Artificial Sequence; identical to SEQ ID NO: 88
  C3-VK

SEQ ID NO: 133 - protein, 119 aa, Artificial Sequence
  C6-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELYFRYALDYWGQGTTVTVSS

SEQ ID NO: 134 - protein, 107 aa, Artificial Sequence; identical to SEQ ID NO: 88
  C6-VK

SEQ ID NO: 135 - protein, 119 aa, Artificial Sequence
  A1-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELLHRYALDYWGQGTTVTVSS

SEQ ID NO: 136 - protein, 107 aa, Artificial Sequence; identical to SEQ ID NO: 88
  A1-VK

SEQ ID NO: 137 - protein, 119 aa, Artificial Sequence
  A2-VH
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELRGRYALDYWGQGTTVTVSS

SEQ ID NO: 138 - protein, 107 aa, Artificial Sequence; identical to SEQ ID NO: 88
  A2-VK

SEQ ID NO: 139 - protein, 119 aa, Artificial Sequence; identical to SEQ ID NO: 123
  A3-VH

SEQ ID NO: 140 - protein, 107 aa, Artificial Sequence
  A3-VK
DIQMTQSPSSLSASVGDRVTITCKASQDVTPAVAWYQQKPGKAPKLLIYSTSSRYTGVPSRFSGSGSGTDFTFTISSLQPEDIATYYCQQHSDAPLTFGQGTKLEIK

SEQ ID NO: 141 - protein, 119 aa, Artificial Sequence; identical to SEQ ID NO: 80
  H12-VH

SEQ ID NO: 142 - protein, 119 aa, Artificial Sequence; identical to SEQ ID NO: 129
  B6-VH

SEQ ID NO: 143 - protein, 107 aa, Artificial Sequence; identical to SEQ ID NO: 88
  H12-VL

SEQ ID NO: 144 - protein, 107 aa, Artificial Sequence; identical to SEQ ID NO: 88
  B6-VL

SEQ ID NO: 145 - DNA, 1338 bp, Artificial Sequence
  Synthetic Heavy chain (full-length) coding gene
gaagttcagctgttggaatctggcggcggattggttcagcctggcggatctctgagactgtcttgtgccgcctctggcttcaccttctccgactacgctatgtcctgggtccgacaggctcctggcaaaggactggaatgggtgtcctccatctcttccggctccggctctatctactacgccgactctgtgaagggcagattcaccatcagccgggacaactccaagaacaccctgtacctgcagatgaactccctgagagccgaggacaccgccgtgtactactgcgccaagaatctgatccctctggactattggggccagggcacactggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccttgccaccttccagagaagagatgaccaagaaccaggtgtccctgacctgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 146 - DNA, 1337 bp, Artificial Sequence
  Synthetic Heavy chain (full-length) coding gene
gaagtccaactcctggagtccggtggtggtctcgttcaacccggaggtagtctccgtctgagttgtgcagcttctggtttcaccttttcaggttactatatgtcctgggtacgccaggcacctggaaaaggcttggaatgggtctcccttattagtccaagcagtggtagtatttactacgctgactctgtaaaaggtcgtttcactatttcaagagacaacagcaagaacacactttacttgcaaatgaatagcctgagggccgaagacaccgccgtctattactgtgccaaaggcttgacaaaatttgattactggggacaaggtacattggtgactgttagctcagcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtcgtgaccgtgccctcctcctccctgggcacccagacctacatctgcaacgtgaaccacaagccctccaacaccaaggtggacaagaaggtggagcccaagtcctgcgacaagacccacacctgccctccctgccccgcccccgagctgctgggcggcccctccgtgttcctgttccctcctaagcccaaggacaccctgatgatctcccggacccccgaggtgacttgctggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaag

SEQ ID NO: 147 - DNA, 1338 bp, Artificial Sequence
  Synthetic Heavy chain (full-length) coding gene
gaggtccagctcctggagagcggaggaggattggttcaacccggaggatcactccgtttgagttgcgcagccagtggattcactttttctagttattcaatgtcctgggttcgtcaggcccccggcaagggattggagtgggtcagcgggatatatagcgatggatcaaatacctattatgctgatagcgtgaaagggcgatttactatatcacgggacaattccaagaatacattgtaccttcagatgaactcccttagggccgaagacactgccgtgtactattgtgcaaagatgcttcatcgttttgattattgggggcaaggaactctggtgactgtctcaagcgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtcgtgaccgtgccctcctcctccctgggcacccagacctacatctgcaacgtgaaccacaagccctccaacaccaaggtggacaagaaggtggagcccaagtcctgcgacaagacccacacctgccctccctgccccgcccccgagctgctgggcggcccctccgtgttcctgttccctcctaagcccaaggacaccctgatgatctcccggacccccgaggtgacttgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaag

SEQ ID NO: 148 - DNA, 1353 bp, Artificial Sequence
  Synthetic Heavy chain (full-length) coding gene
gaagttcagctgttggaatctggcggcggattggttcagcctggcggatctctgagactgtcttgtgccgcctctggcttcaccttctccgactacgctatgtcctgggtccgacaggctcctggcaaaggattggagtgggtgtccggaatttcccctggcggctctaacacctactacgccgattccgtgaagggcagattcaccatcagccgggacaactccaagaacaccctgtacctgcagatgaactccctgagagccgaggacaccgccgtgtactactgtgccaaggatgcctggatcgccagactgctgctgttcgattattggggccagggcacactggtcaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaggtgtacaccctgcctccatctcgggaagagatgaccaagaaccaggtgtccctgacctgcctcgtgaagggattctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 149 - DNA, 1338 bp, Artificial Sequence
  Synthetic Heavy chain (full-length) coding gene
gaggttcagctccttgaatcaggggggggtcttgtccagcccggaggttcccttcgcttgagctgtgcagcatcagggtttaccttcagttcttatgggatgtcttgggtacgtcaggcacctggcaaaggtctcgaatgggtcagtggtatatattctggcggaagcagtaagtactacgccgatagcgtaaaaggtcgtttcaccatctctagggacaattccaagaataccttgtacttgcagatgaacagtctccgagctgaagatacagctgtctactattgtgctaaaaacaggcttcgattcgattattggggccagggtactcttgttactgtcagtagtgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtcgtgaccgtgccctcctcctccctgggcacccagacctacatctgcaacgtgaaccacaagccctccaacaccaaggtggacaagaaggtggagcccaagtcctgcgacaagacccacacctgccctccctgccccgcccccgagctgctgggcggcccctccgtgttcctgttccctcctaagcccaaggacaccctgatgatctcccggacccccgaggtgacttgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaag

SEQ ID NO: 150 - DNA, 1338 bp, Artificial Sequence
  Synthetic Heavy chain coding gene
gaggtccagctcctggagagcggaggaggattggttcaacccggaggatcactccgtttgagttgcgcagccagtggattcactttttctagttattcaatgtcctgggttcgtcaggcccccggcaagggattggagtgggtcagcgggatatatagcgatgcttcaaatacctattatgctgatagcgtgaaagggcgatttactatatcacgggacaattccaagaatacattgtaccttcagatgaactcccttagggccgaagacactgccgtgtactattgtgcaaagatgcttcatcgttttgattattgggggcaaggaactctggtgactgtctcaagcgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtcgtgaccgtgccctcctcctccctgggcacccagacctacatctgcaacgtgaaccacaagccctccaacaccaaggtggacaagaaggtggagcccaagtcctgcgacaagacccacacctgccctccctgccccgcccccgagctgctgggcggcccctccgtgttcctgttccctcctaagcccaaggacaccctgatgatctcccggacccccgaggtgacttgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaag

SEQ ID NO: 151 - DNA, 648 bp, Artificial Sequence
  Synthetic Light chain (full-length) coding gene
cagtctgttctgactcagcctccttctgcttctggcacccctggccagagagtgaccatctcttgttccggctcctcctccaacatcggctctaacgccgtgtcctggtatcagcagttgcctggcacagcccctaagctgctgatctactacaactctcacagaccctccggcgtgcccgacagattctctggctctaagtctggcacctccgccagcctggctatctctggactgagatctgaggacgaggccgactactactgcggctcttgggatgcctctctgaacgcttatgtgttcggcggaggcaccaagctgacagtgttgggacaacctaaggccgctcctagcgtgaccctgtttcctccatcttctgaggaactgcaggccaacaaggctaccctcgtgtgcctgatctctgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctactcttgccaagtgacccatgagggctccaccgtggaaaagacagtggcccctgctgagtgctct

SEQ ID NO: 152 - DNA, 648 bp, Artificial Sequence
  Synthetic Light chain (full-length) coding gene
cagtctgtgcttacccaacctcctagtgcaagtggtacccctggacaacgagtaacaatcagttgcactggtagttcaagtaatataggatctaacgacgtaagttggtatcagcaacttcctggtacagcacctaagttgctcatttacgcaaactcccatagaccctctggcgtccctgatcgtttcagcggtagtaaatccggtacatcagcttccttggctatatctggtctcagatccgaggacgaagctgactattactgtgggagttgggatgactctttgtccggctacgtttttggaggaggcaccaagttgacagtgctgggtcagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccgccgagtgctcc

SEQ ID NO: 153 - DNA, 648 bp, Artificial Sequence
  Synthetic Light chain (full-length) coding gene
caatctgtgctgacccaaccacccagtgcttcaggcacacccggacagagggtgactataagttgcagcgggtcaagttcaaatatcggaagcaattccgtgacctggtaccaacagctccccggtactgcaccaaagctccttatctatgctgattctcagcggcctagtggagtgcctgatcggttcagcggttcaaagtccggtacctccgcttctttggcaataagtggattgcgctccgaggatgaggcagattattattgcgggacatgggatagcagtcttaatgcctacgtattcggcggtggtaccaaacttacagttctcggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccgccgagtgctcc

SEQ ID NO: 154 - DNA, 648 bp, Artificial Sequence
  Synthetic Light chain (full-length) coding gene
cagtctgttctgactcagcctccttctgcttctggcacccctggccagagagtgaccatctcttgttccggctcctcctccaacatcggctctaacgctgtgacctggtatcagcagctgcctggcacagcccctaaactgctgatctactacaacaacaagcggccctctggcgtgcccgacagattctctggatctaagtccggcacctctgccagcctggctatctctggactgagatctgaggacgaggccgactactactgcggcacctgggatgattctctgtccggctatgtgttcggcggaggcacaaaactgacagtgctgggacagcctaaggccgctccttctgtgaccctgtttcctccatcctctgaggaactgcaggccaacaaggctaccctcgtgtgcctgatctctgacttttatcctggcgccgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctactcttgccaagtgacccatgagggctccaccgtggaaaagacagtggcccctgctgagtgctct

SEQ ID NO: 155 - DNA, 648 bp, Artificial Sequence
  Synthetic Light chain (full-length) coding gene
caaagcgttttgactcagcctccttcagcttctggaactccaggacaacgtgtcaccatcagttgcaccggctcttcctccaacatcggaagtaacagcgttacctggtatcagcagctcccaggcactgccccaaagctcttgatatactcagactcccatcgaccatccggagttcctgacagattcagcggttcaaaatctggtacttctgcatcacttgccatttccggtctccgatcagaagacgaagctgactattattgtggaacctgggatgcctcccttaacgcttacgttttcggaggtggcaccaagctcacagttctcggacagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccgccgagtgctcc

SEQ ID NO: 156 - DNA, 648 bp, Artificial Sequence
  Synthetic Light chain coding gene
caatctgtgctgacccaaccacccagtgcttcaggcacacccggacagagggtgactataagttgcagcgggtcaagttcaaatatcggaagcaattccgtgacctggtaccaacagctccccggtactgcaccaaagctccttatctatgctgatgtgcagcggcctagtggagtgcctgatcggttcagcggttcaaagtccggtacctccgcttctttggcaataagtggattgcgctccgaggatgaggcagattattattgcgggacatgggatagcagtcttaatgcctacgtattcggcggtggtaccaaacttacagttctcggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccgccgagtgctcc

SEQ ID NO: 157 - protein, 119 aa, Artificial Sequence; identical to SEQ ID NO: 80
  Synthetic Heavy chain constant region

SEQ ID NO: 158 - DNA, 1001 bp, Artificial Sequence
  Synthetic Heavy chain constant region coding gene
gcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtcgtgaccgtgccctcctcctccctgggcacccagacctacatctgcaacgtgaaccacaagccctccaacaccaaggtggacaagaaggtggagcccaagtcctgcgacaagacccacacctgccctccctgccccgcccccgagctgctgggcggcccctccgtgttcctgttccctcctaagcccaaggacaccctgatgatctcccggacccccgaggtgacttgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgc

SEQ ID NO: 159 - DNA, 990 bp, Artificial Sequence
  Synthetic Heavy chain constant region coding gene
gcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccttgccaccttccagagaagagatgaccaagaaccaggtgtccctgacctgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 160 - DNA, 990 bp, Artificial Sequence
  Synthetic Heavy chain constant region coding gene
gcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaggtgtacaccctgcctccatctcgggaagagatgaccaagaaccaggtgtccctgacctgcctcgtgaagggattctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 161 - protein, 105 aa, Artificial Sequence
  Synthetic Light chain constant region
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 162 - DNA, 315 bp, Artificial Sequence
  Synthetic Light chain constant region coding gene
cagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccgccgagtgctcc

SEQ ID NO: 163 - DNA, 315 bp, Artificial Sequence
  Synthetic Light chain constant region coding gene
caacctaaggccgctcctagcgtgaccctgtttcctccatcttctgaggaactgcaggccaacaaggctaccctcgtgtgcctgatctctgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctactcttgccaagtgacccatgagggctccaccgtggaaaagacagtggcccctgctgagtgctct

SEQ ID NO: 164 - DNA, 315 bp, Artificial Sequence
  Synthetic Light chain constant region coding gene
cagcctaaggccgctccttctgtgaccctgtttcctccatcctctgaggaactgcaggccaacaaggctaccctcgtgtgcctgatctctgacttttatcctggcgccgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctactcttgccaagtgacccatgagggctccaccgtggaaaagacagtggcccctgctgagtgctct

SEQ ID NO: 165 - protein, 446 aa, Artificial Sequence
  Heavy chain of B5 in Heavy component in ABLPNB.01
EVQLLESGGGLVQPGGSLRLSCAASGFTFSDYAMSWVRQAPGKGLEWVSSISSGSGSIYYADSVKGRFTISRDNSKNTLYLQMNSLRAEDTAVYYCAKNLIPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 15 aa, Artificial Sequence
  Linker of Heavy component in ABLPNB.01
GGGGSGGGGSGGGGS

SEQ ID NO: 167 - protein, 108 aa, Artificial Sequence
  VL of scFv of B6 in ABLPNB.01
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGCGTKLEIKR

SEQ ID NO: 168 - protein, 20 aa, Artificial Sequence
  Linker of scFv of B6 in ABLPNB.01
GGGGSGGGGSGGGGSGGGGS

SEQ ID NO: 169 - protein, 119 aa, Artificial Sequence
  VH of scFv of B6 in ABLPNB.01
EVQLVESGGGLVQPGGSLRLSCAASGFTFSSYDMSWVRQAPGKCLEWVATISDAGGYIYYRDSVKGRFTISRDNAKNSLYLQMNSLRDEDTAVYICARELPWRYALDYWGQGTTVTVSS

SEQ ID NO: 170 - protein, 216 aa, Artificial Sequence
  Light component in ABLPNB.01
QSVLTQPPSASGTPGQRVTISCSGSSSNIGSNAVSWYQQLPGTAPKLLIYYNSHRPSGVPDRFSGSKSGTSASLAISGLRSEDEADYYCGSWDASLNAYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 171 - DNA, 2124 bp, Artificial Sequence
  Heavy component in ABLPNB.01
gaagttcagctgttggaatctggcggcggattggttcagcctggcggatctctgagactgtcttgtgccgcctctggcttcaccttctccgactacgctatgtcctgggtccgacaggctcctggcaaaggactggaatgggtgtcctccatctcttccggctccggctctatctactacgccgactctgtgaagggcagattcaccatcagccgggacaactccaagaacaccctgtacctgcagatgaactccctgagagccgaggacaccgccgtgtactactgcgccaagaatctgatccctctggactattggggccagggcacactggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctaccgggtggtgtccgtgctgaccgttctgcaccaggattggctgaacggcaaagagtacaagtgcaaggtgtccaacaaggccctgcctgcccctatcgaaaagaccatctctaaggccaagggccagccccgggaacctcaagtgtacaccttgcctcccagccgggaagagatgaccaagaaccaggtgtccctgacctgcctggttaagggcttctacccctccgatatcgccgtggaatgggagtctaatggccagcctgagaacaactacaagacaacccctcctgtgctggactccgacggctcattcttcctgtactccaagctgaccgtggacaagtccagatggcagcagggcaacgtgttctcctgctccgtgatgcacgaggctctgcacaaccactacacccagaagtccctgtctctgtcccctggaaaaggcggcggaggatctggcggaggtggaagcggaggcggtggatctgacatccagatgacccagtctccatccagcctgtctgcttccgtgggcgacagagtgaccatcacatgcaaggccagccaggatgtgacccctgccgttgcctggtatcaacagaagcctggcaaggcccctaagctgctgatctactccacctccagcagatacacaggcgtgccctccagattctccggctctggctctggcaccgactttacctttacaatttccagcctgcagcctgaggacattgccacctactactgccagcagcactacaccacacctctgacctttggctgcggcaccaagctggaaatcaagagaggtggcggaggtagcggtggtggtggtagtggcggaggcggctctggtggtggcggatctgaagtgcagttggtggaatctggcggcggattggttcagccaggcggatctctgagactgtcttgtgccgccagcggcttcaccttctcctcttacgacatgtcctgggtccgacaggccccaggcaaatgtctggaatgggtcgccaccatctctgatgctggcggctacatctattaccgggactccgtgaagggcagattcaccatcagccgggacaatgccaagaactccctgtacctgcagatgaactctctgcgcgacgaggacaccgctgtgtacatctgtgctagagagctgccttggagatacgccctggactactggggacagggaactaccgtgactgtgtcctcc

SEQ ID NO: 172 - DNA, 648 bp, Artificial Sequence; identical to SEQ ID NO: 151
  Light component in ABLPNB.01

SEQ ID NO: 173 - protein, 446 aa, Artificial Sequence
  Heavy chain of C41 in Heavy component in ABLPNB.02
EVQLLESGGGLVQPGGSLRLSCAASGFTFSGYYMSWVRQAPGKGLEWVSLISPSSGSIYYADSVKGRFTISRDNSKNTLYLQMNSLRAEDTAVYYCAKGLTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 15 aa, Artificial Sequence
  Linker of Heavy component in ABLPNB.02
GGGGSGGGGSGGGGS

SEQ ID NO: 175 - protein, 108 aa, Artificial Sequence; identical to SEQ ID NO: 167
  VL of scFv of B6 in ABLPNB.02

SEQ ID NO: 176 - protein, 20 aa, Artificial Sequence
  Linker of scFv of B6 in ABLPNB.02
GGGGSGGGGSGGGGSGGGGS

SEQ ID NO: 177 - protein, 119 aa, Artificial Sequence; identical to SEQ ID NO: 169
  VH of scFv of B6 in ABLPNB.02

SEQ ID NO: 178 - protein, 216 aa, Artificial Sequence
  Light component in ABLPNB.02
QSVLTQPPSASGTPGQRVTISCTGSSSNIGSNDVSWYQQLPGTAPKLLIYANSHRPSGVPDRFSGSKSGTSASLAISGLRSEDEADYYCGSWDDSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 179 - DNA, 2124 bp, Artificial Sequence
  Heavy component in ABLPNB.02
gaagttcagctgctggaatctggcggcggattggttcaacctggcggctctctgagactgtcttgtgccgcttctggcttcaccttctccggctactacatgtcctgggtccgacaggctcctggcaaaggactggaatgggtgtccctgatctccccttcctccggctctatctactacgccgactccgtgaagggcagattcaccatctctcgggacaactccaagaacaccctgtacctgcagatgaactccctgagagccgaggacaccgccgtgtactattgtgctaagggcctgaccaagttcgactactggggccagggaaccctggtcacagtttcctctgctagcaccaagggcccctctgtgttccctctggccccttcctctaaatccacctctggcggaaccgctgctctgggctgtctggtcaaggactacttccctgagcccgtgaccgtgtcttggaattctggcgctctgaccagcggagtgcacacctttccagctgtgctgcagtcctccggcctgtactctctgtcctctgtcgtgacagtgccttccagctctctgggcacccagacctacatctgcaacgtgaaccacaagccctccaacaccaaggtggacaagaaggtggaacccaagtcctgcgacaagacccacacctgtcctccatgtcctgctccagaactgctgggcggaccctccgtgttcctgttccctccaaagcctaaggacaccctgatgatctcccggacccctgaagtgacctgcgtggtggtggatgtgtcccacgaggatcccgaagtgaagttcaattggtacgtggacggcgtggaagtgcacaacgccaagaccaagcctagagaggaacagtacaactccacctaccgggtggtgtccgtgctgaccgttctgcaccaggattggctgaacggcaaagagtacaagtgcaaggtgtccaacaaggccctgcctgcccctatcgaaaagaccatctctaaggccaagggccagccccgggaacctcaagtgtacaccttgcctcccagccgggaagagatgaccaagaaccaggtgtccctgacctgcctggttaagggcttctacccctccgatatcgccgtggaatgggagtctaatggccagcctgagaacaactacaagacaacccctcctgtgctggactccgacggctcattcttcctgtactccaagctgaccgtggacaagtccagatggcagcagggcaacgtgttctcctgctccgtgatgcacgaggctctgcacaaccactacacccagaagtccctgtctctgtcccctggaaaaggcggcggaggatctggcggaggtggaagcggaggcggtggatctgacatccagatgacccagtctccatccagcctgtctgcttccgtgggcgacagagtgaccatcacatgcaaggccagccaggatgtgacccctgccgttgcctggtatcaacagaagcctggcaaggcccctaagctgctgatctactccacctccagcagatacacaggcgtgccctccagattctccggctctggctctggcaccgactttacctttacaatttccagcctgcagcctgaggacattgccacctactactgccagcagcactacaccacacctctgacctttggctgcggcaccaagctggaaatcaagagaggtggcggaggtagcggtggtggtggtagtggcggaggcggctctggtggtggcggatctgaagtgcagttggtggaatctggcggcggattggttcagccaggcggatctctgagactgtcttgtgccgccagcggcttcaccttctcctcttacgacatgtcctgggtccgacaggccccaggcaaatgtctggaatgggtcgccaccatctctgatgctggcggctacatctattaccgggactccgtgaagggcagattcaccatcagccgggacaatgccaagaactccctgtacctgcagatgaactctctgcgcgacgaggacaccgctgtgtacatctgtgctagagagctgccttggagatacgccctggactactggggacagggaactaccgtgactgtgtcctcc

SEQ ID NO: 180 - DNA, 648 bp, Artificial Sequence; identical to SEQ ID NO: 152
  Light component in ABLPNB.02

SEQ ID NO: 181 - protein, 449 aa, Artificial Sequence
  Heavy chain of B6 in Heavy component in ABLPNB.03
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 15 aa, Artificial Sequence
  Linker of Heavy component in ABLPNB.03
GGGGSGGGGSGGGGS

SEQ ID NO: 183 - protein, 110 aa, Artificial Sequence
  VL of scFv of B5 in ABLPNB.03
QSVLTQPPSASGTPGQRVTISCSGSSSNIGSNAVSWYQQLPGTAPKLLIYYNSHRPSGVPDRFSGSKSGTSASLAISGLRSEDEADYYCGSWDASLNAYVFGCGTKLTVL

SEQ ID NO: 184 - protein, 20 aa, Artificial Sequence
  Linker of scFv of B5 in ABLPNB.03
GGGGSGGGGSGGGGSGGGGS

SEQ ID NO: 185 - protein, 116 aa, Artificial Sequence
  VH of scFv of B5 in ABLPNB.03
EVQLLESGGGLVQPGGSLRLSCAASGFTFSDYAMSWVRQAPGKCLEWVSSISSGSGSIYYADSVKGRFTISRDNSKNTLYLQMNSLRAEDTAVYYCAKNLIPLDYWGQGTLVTVSS

SEQ ID NO: 186 - protein, 214 aa, Artificial Sequence
  Light component in ABLPNB.03
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - DNA, 2130 bp, Artificial Sequence
  Heavy component in ABLPNB.03
gaagtgcagctggttgaatctggcggcggattggttcagcctggcggatctctgagactgtcttgtgccgcctccggcttcaccttctccagctacgatatgtcctgggtccgacaggcccctggcaagtctttggaatgggtcgccaccatctctgacgctggcggctacatctactaccgggactctgtgaagggcagattcaccatcagccgggacaacgccaagaactccctgtacctgcagatgaacagcctgcgcgacgaggataccgccgtgtacatctgtgctagagagctgccttggagatacgccctggattattggggccagggcaccacagtgaccgtgtcctctgcttctaccaagggacccagcgtgttccctctggctccttccagcaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgaccgtgccttccagctctctgggcacccagacctacatctgcaacgtgaaccacaagccctccaacaccaaggtggacaagaaggtggaacccaagtcctgcgacaagacccacacctgtcctccatgtcctgctccagaactgctgggcggaccctccgtgttcctgttccctccaaagcctaaggacaccctgatgatctcccggacccctgaagtgacctgcgtggtggtggatgtgtcccacgaggatcccgaagtgaagttcaattggtacgtggacggcgtggaagtgcacaacgccaagaccaagcctagagaggaacagtacgcctccacctaccgggtggtgtccgtgctgaccgttctgcaccaggattggctgaacggcaaagagtacaagtgcaaggtgtccaacaaggccctgcctgcccctatcgaaaagaccatctctaaggccaagggccagccccgggaacctcaagtgtacaccttgcctcccagccgggaagagatgaccaagaaccaggtgtccctgacctgcctggttaagggcttctacccctccgatatcgccgtggaatgggagtccaatggccagcctgagaacaactacaagaccacacctcctgtgctggactccgacggctcattcttcctgtactccaagctgaccgtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctcagtctgttctgacccagcctccttccgcttctggcacacctggacagagagtgaccatctcttgctccggctcctcctccaacatcggctctaatgccgtgtcctggtatcagcagctgcctggcacagctcccaaactgctgatctactacaactcccaccggccttccggcgtgcccgatagattttccggctctaagtccggcacctctgccagcctggctatctctggcctgagatctgaggacgaggccgactactactgcggctcttgggatgcctctctgaacgcctacgtgttcggctgtggcacaaagctgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctctggcttcaccttctccgactacgctatgtcttgggtccgacaggcccctggcaagtgtctggaatgggtttcctccatctcctccggcagcggctctatctactacgccgactctgtgaagggcagattcaccatcagccgggacaactccaagaacaccctgtacctgcagatgaactccctgagagccgaggacaccgccgtgtactactgtgccaagaatctgatccctctggactactggggccagggcacactggttacagtgtcctct

SEQ ID NO: 188 - DNA, 642 bp, Artificial Sequence
  Light component in ABLPNB.03
gacatccagatgacccagagccctagcagcctgagcgctagcgtgggcgacagggtgaccatcacctgcaaggccagccaggatgtgacccctgccgtggcctggtaccagcagaagcccggcaaggcccccaagctgctgatctacagcaccagcagcaggtacaccggcgtgcccagcaggtttagcggaagcggcagcggcaccgacttcaccttcaccatcagcagcctgcagcccgaggacatcgccacctactactgccagcagcactacaccacccctctgaccttcggccagggcaccaagctggagatcaagagaaccgtggccgctccctccgtgttcatcttcccaccatctgacgagcagctgaagtccggcaccgcttctgtcgtgtgcctgctgaacaacttctaccctcgggaagccaaggtgcagtggaaggtggacaatgccctgcagtccggcaactcccaagagtctgtgaccgagcaggactccaaggacagcacctactccctgtcctctaccctgaccctgtccaaggccgactacgagaagcacaaggtgtacgcctgcgaagtgacccaccagggactgtctagccccgtgaccaagtccttcaacagaggcgagtgc

SEQ ID NO: 189 - protein, 449 aa, Artificial Sequence; identical to SEQ ID NO: 181
  Heavy chain of B6 in Heavy component in ABLPNB.04

SEQ ID NO: 190 - protein, 15 aa, Artificial Sequence
  Linker of Heavy component in ABLPNB.04
GGGGSGGGGSGGGGS

SEQ ID NO: 191 - protein, 110 aa, Artificial Sequence
  VL of scFv of C4I in ABLPNB.04
QSVLTQPPSASGTPGQRVTISCTGSSSNIGSNDVSWYQQLPGTAPKLLIYANSHRPSGVPDRFSGSKSGTSASLAISGLRSEDEADYYCGSWDDSLSGYVFGCGTKLTVL

SEQ ID NO: 192 - protein, 20 aa, Artificial Sequence
  Linker of scFv of C4I in ABLPNB.04
GGGGSGGGGSGGGGSGGGGS

SEQ ID NO: 193 - protein, 116 aa, Artificial Sequence
  VH of scFv of C4I in ABLPNB.04
EVQLLESGGGLVQPGGSLRLSCAASGFTFSGYYMSWVRQAPGKCLEWVSLISPSSGSIYYADSVKGRFTISRDNSKNTLYLQMNSLRAEDTAVYYCAKGLTKFDYWGQGTLVTVSS

SEQ ID NO: 194 - protein, 214 aa, Artificial Sequence; identical to SEQ ID NO: 186
  Light component in ABLPNB.04

SEQ ID NO: 195 - DNA, 2130 bp, Artificial Sequence
  Heavy component in ABLPNB.04
gaagtgcagctggttgaatctggcggcggattggttcagcctggcggatctctgagactgtcttgtgccgcctccggcttcaccttctccagctacgatatgtcctgggtccgacaggcccctggcaagtctttggaatgggtcgccaccatctctgacgctggcggctacatctactaccgggactctgtgaagggcagattcaccatcagccgggacaacgccaagaactccctgtacctgcagatgaacagcctgcgcgacgaggataccgccgtgtacatctgtgctagagagctgccttggagatacgccctggattattggggccagggcaccacagtgaccgtgtcctctgcttctaccaagggacccagcgtgttccctctggctccttccagcaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgaccgtgccttccagctctctgggcacccagacctacatctgcaacgtgaaccacaagccctccaacaccaaggtggacaagaaggtggaacccaagtcctgcgacaagacccacacctgtcctccatgtcctgctccagaactgctgggcggaccctccgtgttcctgttccctccaaagcctaaggacaccctgatgatctcccggacccctgaagtgacctgcgtggtggtggatgtgtcccacgaggatcccgaagtgaagttcaattggtacgtggacggcgtggaagtgcacaacgccaagaccaagcctagagaggaacagtacgcctccacctaccgggtggtgtccgtgctgaccgttctgcaccaggattggctgaacggcaaagagtacaagtgcaaggtgtccaacaaggccctgcctgcccctatcgaaaagaccatctctaaggccaagggccagccccgggaacctcaagtgtacaccttgcctcccagccgggaagagatgaccaagaaccaggtgtccctgacctgcctggttaagggcttctacccctccgatatcgccgtggaatgggagtccaatggccagcctgagaacaactacaagaccacacctcctgtgctggactccgacggctcattcttcctgtactccaagctgaccgtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctcagtctgttctgacccagcctccttccgcttctggcacacctggacagagagtgaccatctcttgcaccggctcctccagcaacatcggctctaacgacgtgtcctggtatcagcagctgcctggcacagctcccaaactgctgatctacgccaacagccacagaccttccggcgtgcccgatagattctccggctctaagtctggcacctctgccagcctggctatctccggcctgagatctgaggacgaggccgactactactgcggctcttgggacgattccctgtccggctatgtgttcggctgtggcacaaagctgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctctggcttcaccttcagcggctactacatgtcttgggtccgacaggcccctggcaagtgtctggaatgggtttccctgatctcccctagctccggctccatctactacgccgactctgtgaagggcagattcaccatcagccgggacaactccaagaacaccctgtacctgcagatgaactccctgagagccgaggacaccgccgtgtactattgtgctaagggcctgaccaagttcgactactggggccagggaaccctggtcacagtctcttct

SEQ ID NO: 196 - DNA, 642 bp, Artificial Sequence; identical to SEQ ID NO: 188
  Light component in ABLPNB.04

SEQ ID NO: 197 - protein, 446 aa, Artificial Sequence
  Heavy chain of (HC+LC) of B5 in ABLPNB.05
EVQLLESGGGLVQPGGSLRLSCAASGFTFSDYAMSWVRQAPGKGLEWVSSISSGSGSIYYADSVKGRFTISRDNSKNTLYLQMNSLRAEDTAVYYCAKNLIPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 216 aa, Artificial Sequence; identical to SEQ ID NO: 170
  Light chain of (HC+LC) of B5 in ABLPNB.05

SEQ ID NO: 199 - protein, 727 aa, Artificial Sequence
  scFab-Fc of B6 in ABLPNB.05
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGSGSGSGSGSGSGSGSGGGGSGGGGSGGGGSGGGGSGGGGSGGGGSGSGSGSGSGSGSGSGSEVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 200 - protein, 108 aa, Artificial Sequence
  VL of scFab-Fc of B6 in ABLPNB.05
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIKR

SEQ ID NO: 201 - protein, 64 aa, Artificial Sequence
  Linker of scFab-Fc of B6
GSGSGSGSGSGSGSGSGSGGGGSGGGGSGGGGSGGGGSGGGGSGGGGSGSGSGSGSGSGSGSGS

SEQ ID NO: 202 - protein, 119 aa, Artificial Sequence; identical to SEQ ID NO: 129
  VH of scFab-Fc of B6 in ABLPNB.05

SEQ ID NO: 203 - DNA, 1338 bp, Artificial Sequence
  Heavy Chain in ABLPNB.05
gaagttcagctgttggaatctggcggcggattggttcagcctggcggatctctgagactgtcttgtgccgcctctggcttcaccttctccgactacgctatgtcctgggtccgacaggctcctggcaaaggactggaatgggtgtcctccatctcttccggctccggctctatctactacgccgactctgtgaagggcagattcaccatcagccgggacaactccaagaacaccctgtacctgcagatgaactccctgagagccgaggacaccgccgtgtactactgcgccaagaatctgatccctctggactattggggccagggcacactggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccttgccaccttccagagaagagatgaccaagaaccaggtgtccctgtggtgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 204 - DNA, 648 bp, Artificial Sequence; identical to SEQ ID NO: 151
  Light Chain in ABLPNB.05

SEQ ID NO: 205 - DNA, 2181 bp, Artificial Sequence
  scFab-Fc of B6 in ABLPNB.05
gatatccagatgacccagtctccttccagcctgtctgcctctgtgggcgacagagtgaccatcacatgcaaggccagccaggatgtgacccctgccgttgcttggtatcagcagaagcctggcaaggcccctaagctgctgatctactccacctcctccagatacacaggcgtgccctccagattctccggctctggctctggcaccgactttacctttacaatctccagcctgcagcctgaggatatcgccacctactactgccagcagcactacaccacacctctgacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctatagcctgtcctccacactgaccctgtccaaggccgactacgagaagcacaaggtgtacgcctgcgaagtgacccatcagggcctgtctagtcccgtgaccaagtctttcaacagaggcgagtgtggctccggcagcggaagtggttctggaagcggatctggttccggaagtggcagcggaggcggaggatctggtggcggaggaagtggcggaggcggtagtggtggtggtggatcaggtggtggcggatccggcggaggtggtagcggttctggttcaggatctggatctggcagcggctccggttctggatccgaagtgcagctggttgaatctggcggtggactggttcagcctggcggatctctgagactgtcttgtgccgcctccggcttcaccttctccagctacgatatgtcctgggtccgacaggcccctggcaagtctttggaatgggtcgccaccatctctgacgctggcggctacatctactaccgggactctgtgaagggcagattcaccatcagccgggacaacgccaagaactccctgtacctgcagatgaacagcctgcgcgacgaggacaccgccgtgtatatctgtgctagagagctgccttggagatacgccctggattattggggccagggaaccacggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccttgcctccaagccgggaagagatgaccaagaaccaggtgtccctgtcctgtgccgtgaagggcttctacccctctgatatcgccgtggaatgggagagcaatggccagcctgagaacaactacaagacaacccctcctgtgctggactccgatggctcattcttcctggtgtccaagctgactgtggacaagtccagatggcagcagggcaacgtgttctcctgctccgtgatgcacgaggccctgcacaatcactacacccagaagtccctgtctctgagcccaggcaaa

SEQ ID NO: 206 - protein, 446 aa, Artificial Sequence; identical to SEQ ID NO: 197
  Heavy chain of (HC+LC) of B5 in ABLPNB.06

SEQ ID NO: 207 - protein, 216 aa, Artificial Sequence; identical to SEQ ID NO: 170
  Light chain of (HC+LC) of B5 in ABLPNB.06

SEQ ID NO: 208 - protein, 727 aa, Artificial Sequence
  scFab-Fc of B6 in ABLPNB.06
DIQMTQSPSTLSASVGDRVIITCRASRGISSWLAWYQQKPGKAPNLLISKASSLESGVPSRFSGSGSGTDFTLTISSLQPEDFATYYCQQSSSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGSGSGSGSGSGSGSGSGGGGSGGGGSGGGGSGGGGSGGGGSGGGGSGSGSGSGSGSGSGSGSQVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 209 - protein, 107 aa, Artificial Sequence
  VL of scFab-Fc of B12 in ABLPNB.06
DIQMTQSPSTLSASVGDRVIITCRASRGISSWLAWYQQKPGKAPNLLISKASSLESGVPSRFSGSGSGTDFTLTISSLQPEDFATYYCQQSSSIPLTFGGGTKVEIK

SEQ ID NO: 210 - protein, 64 aa, Artificial Sequence; identical to SEQ ID NO: 201
  Linker of scFab-Fc of B12 in ABLPNB.06

SEQ ID NO: 211 - protein, 119 aa, Artificial Sequence
  VH of scFab-Fc of B12 in ABLPNB.06
QVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSS

SEQ ID NO: 212 - DNA, 1338 bp, Artificial Sequence; identical to SEQ ID NO: 203
  Heavy Chain in ABLPNB.06

SEQ ID NO: 213 - DNA, 648 bp, Artificial Sequence; identical to SEQ ID NO: 151
  Light Chain in ABLPNB.06

SEQ ID NO: 214 - DNA, 2181 bp, Artificial Sequence
  scFab-Fc of B12 in ABLPNB.06
gatatccagatgacccagtctccttccacactgtccgcctctgtgggcgacagagtgatcatcacctgtagagccagccggggcatctcttcttggctggcttggtatcagcagaagcccggcaaggcccctaacctgctgatctctaaggcctcctctctggaatccggcgtgcccagcagattttccggctctggctctggcaccgactttaccctgacaatctccagcctgcagcctgaggacttcgccacctactactgccagcagtccagcagcatccctctgacatttggcggaggcaccaaggtggaaatcaagcggacagtcgccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaacagaggcgagtgcggctccggaagcggaagtggatcaggttctggatctggcagcggtagcggcagtggtggcggaggttctggcggtggcggatcaggcggcggaggaagcggaggcggaggcagtggcggaggtggaagtggcggaggcggatctggaagtggaagcggttctggctcaggatctggttcaggctctggaagccaggtccagctgttggaatctggcggaggacttgttcagcctggcggctctctgagactgtcttgtgccgcttctggcttcaccttctccagctactggatgtcctgggtccgacaggctcctggcaaaggactggaatgggtcgccaacatcaagcaggacggctccgagaagtactacgtggactccgtgaagggcagattcaccatctctcgggacaacgccaagaactccctgtacctgcagatgaacagcctgagagccgaggacaccgccgtgtactactgtgctagagtggccctgtgggacgacgcctttgatatctggggacagggcacaatggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccttgcctccaagccgggaagagatgaccaagaaccaggtgtccctgtcctgtgccgtgaagggcttctacccctctgatatcgccgtggaatgggagagcaatggccagcctgagaacaactacaagacaacccctcctgtgctggactccgatggctcattcttcctggtgtccaagctgactgtggacaagtccagatggcagcagggcaacgtgttctcctgctccgtgatgcacgaggccctgcacaatcactacacccagaagtccctgtctctgagcccaggcaaa

SEQ ID NO: 215 - protein, 446 aa, Artificial Sequence
  Heavy chain of (HC+LC) of C4I in ABLPNB.07
EVQLLESGGGLVQPGGSLRLSCAASGFTFSGYYMSWVRQAPGKGLEWVSLISPSSGSIYYADSVKGRFTISRDNSKNTLYLQMNSLRAEDTAVYYCAKGLTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 216 aa, Artificial Sequence; identical to SEQ ID NO: 178
  Light chain of (HC+LC) of C4I in ABLPNB.07

SEQ ID NO: 217 - protein, 727 aa, Artificial Sequence; identical to SEQ ID NO: 199
  scFab-Fc of B6 in ABLPNB.07

SEQ ID NO: 218 - protein, 108 aa, Artificial Sequence; identical to SEQ ID NO: 200
  VL of scFab-Fc of B6 in ABLPNB.07

SEQ ID NO: 219 - protein, 64 aa, Artificial Sequence; identical to SEQ ID NO: 201
  Linker of scFab-Fc of B6 in ABLPNB.07

SEQ ID NO: 220 - protein, 119 aa, Artificial Sequence; identical to SEQ ID NO: 129
  VH of scFab-Fc of B6 in ABLPNB.07

SEQ ID NO: 221 - DNA, 1338 bp, Artificial Sequence
  Heavy Chain in ABLPNB.07
gaagttcagctgttggaatctggcggcggattggttcagcctggcggatctctgagactgtcttgtgccgcctctggcttcaccttctccggttactatatgtcctgggtccgacaggctcctggcaaaggactggaatgggtgtcccttattagtccaagctccggctctatctactacgccgactctgtgaagggcagattcaccatcagccgggacaactccaagaacaccctgtacctgcagatgaactccctgagagccgaggacaccgccgtgtactactgcgccaagggcttgacaaaatttgactattggggccagggcacactggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccttgccaccttccagagaagagatgaccaagaaccaggtgtccctgtggtgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 222 - DNA, 648 bp, Artificial Sequence; identical to SEQ ID NO: 152
  Light Chain in ABLPNB.07

SEQ ID NO: 223 - DNA, 2181 bp, Artificial Sequence; identical to SEQ ID NO: 205
  scFab-Fc of B6 in ABLPNB.07

SEQ ID NO: 224 - protein, 446 aa, Artificial Sequence; identical to SEQ ID NO: 215
  Heavy chain of (HC+LC) of C4I in ABLPNB.08

SEQ ID NO: 225 - protein, 216 aa, Artificial Sequence; identical to SEQ ID NO: 178
  Light chain of (HC+LC) of C4I in ABLPNB.08

SEQ ID NO: 226 - protein, 727 aa, Artificial Sequence; identical to SEQ ID NO: 208
  scFab-Fc of B12 in ABLPNB.08

SEQ ID NO: 227 - protein, 107 aa, Artificial Sequence; identical to SEQ ID NO: 209
  VL of scFab-Fc of B12 in ABLPNB.08

SEQ ID NO: 228 - protein, 64 aa, Artificial Sequence; identical to SEQ ID NO: 201
  Linker of scFab-Fc of B12 in ABLPNB.08

SEQ ID NO: 229 - protein, 119 aa, Artificial Sequence; identical to SEQ ID NO: 211
  VH of scFab-Fc of B12 in ABLPNB.08

SEQ ID NO: 230 - DNA, 1338 bp, Artificial Sequence; identical to SEQ ID NO: 221
  Heavy Chain in ABLPNB.08

SEQ ID NO: 231 - DNA, 648 bp, Artificial Sequence; identical to SEQ ID NO: 152
  Light Chain in ABLPNB.08

SEQ ID NO: 232 - DNA, 2181 bp, Artificial Sequence; identical to SEQ ID NO: 214
  scFab-Fc of B12 in ABLPNB.08

SEQ ID NO: 233 - protein, 449 aa, Artificial Sequence
  Heavy chain of (HC+LC) of B6 in ABLPNB.09
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 214 aa, Artificial Sequence; identical to SEQ ID NO: 186
  Light chain of (HC+LC) of B6 in ABLPNB.09

SEQ ID NO: 235 - protein, 726 aa, Artificial Sequence
  scFab-Fc of C4I in ABLPNB.09
QSVLTQPPSASGTPGQRVTISCTGSSSNIGSNDVSWYQQLPGTAPKLLIYANSHRPSGVPDRFSGSKSGTSASLAISGLRSEDEADYYCGSWDDSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECSGSGSGSGSGSGSGSGSGSGGGGSGGGGSGGGGSGGGGSGGGGSGGGGSGSGSGSGSGSGSGSGSEVQLLESGGGLVQPGGSLRLSCAASGFTFSGYYMSWVRQAPGKGLEWVSLISPSSGSIYYADSVKGRFTISRDNSKNTLYLQMNSLRAEDTAVYYCAKGLTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 236 - protein, 110 aa, Artificial Sequence; identical to SEQ ID NO: 58
  VL of scFab-Fc of C4I in ABLPNB.09

SEQ ID NO: 237 - protein, 64 aa, Artificial Sequence; identical to SEQ ID NO: 201
  Linker of scFab-Fc of C4I in ABLPNB.09

SEQ ID NO: 238 - protein, 116 aa, Artificial Sequence; identical to SEQ ID NO: 52
  VH of scFab-Fc of C4I in ABLPNB.09

SEQ ID NO: 239 - DNA, 1347 bp, Artificial Sequence
  Heavy Chain in ABLPNB.09
gaagtgcagctggttgaatctggcggcggattggttcagcctggcggatctctgagactgtcttgtgccgcctccggcttcaccttctccagctacgatatgtcctgggtccgacaggcccctggcaagtctttggaatgggtcgccaccatctctgacgctggcggctacatctactaccgggactctgtgaagggcagattcaccatcagccgggacaacgccaagaactccctgtacctgcagatgaacagcctgcgcgacgaggacaccgccgtgtatatctgtgctagagagctgccttggagatacgccctggattattggggccagggcacaacggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccttgccaccttccagagaagagatgaccaagaaccaggtgtccctgtggtgcctcgtgaagggcttctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacccagaagtccctgtctctgtcccctggcaaa

SEQ ID NO: 240 - DNA, 642 bp, Artificial Sequence; identical to SEQ ID NO: 188
  Light Chain in ABLPNB.09

SEQ ID NO: 241 - DNA, 2178 bp, Artificial Sequence
  scFab-Fc of C4I in ABLPNB.09
cagtctgttctgacccagcctccttccgcttctggcacacctggacagagagtgaccatctcttgcaccggctcctccagcaacatcggctctaacgacgtgtcctggtatcagcagctgcctggcacagctcccaaactgctgatctacgccaacagccacagaccttccggcgtgcccgatagattctccggctctaagtctggcacctctgccagcctggctatctccggactgagatctgaggacgaggccgactactactgcggctcttgggacgattccctgtccggctatgttttcggcggaggcaccaagctgacagtgctgggacaacctaaggccgctccttccgtgacactgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagacagtggcccctgctgagtgttccggctctggtagtggttctggctccggaagcggatctggctctggttctggatctggtggcggaggatctggcggaggtggaagcggaggcggaggaagtggtggcggcggaagcggcggtggtggctcaggcggtggcggtagcggcagtggatcaggatctggaagtggcagcggctctggatcaggttccgaagtgcagctgttggagtcaggtggtggactggttcagcctggcggatccctgagactgtcttgtgctgcctctggcttcaccttcagcggctactacatgtcttgggtccgacaggctcccggcaaaggactggaatgggtgtccctgatcagcccctcctctggctctatctactacgccgactccgtgaagggcagattcaccatctctcgggacaactccaagaacaccctgtacctgcagatgaactccctgagagccgaggacaccgccgtgtactattgtgctaagggcctgaccaagttcgactactggggccagggaacactggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccttgcctccaagccgggaagagatgaccaagaaccaggtgtccctgtcctgtgccgtgaagggcttctacccctctgatatcgccgtggaatgggagagcaatggccagcctgagaacaactacaagacaacccctcctgtgctggactccgatggctcattcttcctggtgtccaagctgactgtggacaagtccagatggcagcagggcaacgtgttctcctgctccgtgatgcacgaggccctgcacaatcactacacccagaagtccctgtctctgagcccaggcaaa

SEQ ID NO: 242 - protein, 446 aa, Artificial Sequence; identical to SEQ ID NO: 215
  Heavy chain of (HC+LC) of C4I in Trispecific antibody 01

SEQ ID NO: 243 - protein, 15 aa, Artificial Sequence
  Linker in Trispecific antibody 01
GGGGSGGGGSGGGGS

SEQ ID NO: 244 - protein, 251 aa, Artificial Sequence
  scFv of 1A10 in Trispecific antibody 01
QSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 245 - protein, 216 aa, Artificial Sequence; identical to SEQ ID NO: 178
  Light chain of (HC+LC) of C4I in Trispecific antibody 01

SEQ ID NO: 246 - protein, 993 aa, Artificial Sequence
  scFab-Fc of B6+scFv of 1A10 in Trispecific antibody 01
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGSGSGSGSGSGSGSGSGGGGSGGGGSGGGGSGGGGSGGGGSGGGGSGSGSGSGSGSGSGSGSEVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 247 - protein, 214 aa, Artificial Sequence; identical to SEQ ID NO: 186
  Light chain of B6 in Trispecific antibody 01

SEQ ID NO: 248 - protein, 64 aa, Artificial Sequence; identical to SEQ ID NO: 201
  Linker in Trispecific antibody 01

SEQ ID NO: 249 - protein, 449 aa, Artificial Sequence
  Heavy chain of B6 in Trispecific antibody 01
EVQLVESGGGLVQPGGSLRLSCAASGFTFSSYDMSWVRQAPGKSLEWVATISDAGGYIYYRDSVKGRFTISRDNAKNSLYLQMNSLRDEDTAVYICARELPWRYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 250 - protein, 15 aa, Artificial Sequence
  Linker in Trispecific antibody 01
GGGGSGGGGSGGGGS

SEQ ID NO: 251 - protein, 251 aa, Artificial Sequence; identical to SEQ ID NO: 244
  scFv of 1A10 in Trispecific antibody 01

SEQ ID NO: 252 - DNA, 1338 bp, Artificial Sequence
  Heavy chain of (HC+LC) of C4I in Trispecific antibody 01
gaagttcagctgttggaatctggcggcggattggttcagcctggcggatctctgagactgtcttgtgccgcctctggcttcaccttctccggttactatatgtcctgggtccgacaggctcctggcaaaggactggaatgggtgtcccttattagtccaagctccggctctatctactacgccgactctgtgaagggcagattcaccatcagccgggacaactccaagaacaccctgtacctgcagatgaactccctgagagccgaggacaccgccgtgtactactgcgccaagggcttgacaaaatttgactattggggccagggcacactggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccctgcctcctagcagagaagagatgaccaagaaccaggtgtccctgtggtgcctggtcaagggcttctacccttccgatatcgccgtggaatgggagtccaatggccagcctgagaacaactacaagaccacacctcctgtgctggactccgacggctcattcttcctgtactccaagctgaccgtggacaagtccagatggcagcagggcaacgtgttctcctgctccgtgatgcacgaggccctgcacaatcactacacccagaagtccctgtctctgtcccctggaaaa

SEQ ID NO: 253 - DNA, 45 bp, Artificial Sequence
  Linker in Trispecific antibody 01
ggcggcggaggatctggcggaggtggaagcggaggcggtggatct

SEQ ID NO: 254 - DNA, 753 bp, Artificial Sequence
  scFv of 1A10 in Trispecific antibody 01
cagtctgttctgacccagcctccttccgcttctggcacacctggacagagagtgaccatctcttgctccggctcctcctccaacatcggcaacaactacgtgacctggtatcagcagctgcccggcacagctcccaaactgctgatctacgccgactctcacagaccttccggcgtgcccgatagattctccggctctaagtctggcacctctgccagcctggctatctccggcctgagatctgaggacgaggccgactactactgcgccacctgggattattccctgtccggctacgtgttcggctgcggcacaaaactgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctccggctttaccttctcctcctacgacatgtcttgggtccgacaggcccctggcaagtgtctggaatgggtttcctggatctcctactccggcggctccatctactacgccgattccgtgaagggcagattcaccatcagccgggacaactccaagaacaccctgtacctgcagatgaactccctgagagccgaggacaccgccgtgtactactgtgccagagatgcccagcggaacagcatgagagagttcgactattggggccagggcaccctggtcacagtctcttct

SEQ ID NO: 255 - DNA, 648 bp, Artificial Sequence; identical to SEQ ID NO: 152
  Light chain of (HC+LC) of C4I in Trispecific antibody 01

SEQ ID NO: 256 - DNA, 2979 bp, Artificial Sequence
  scFab-Fc of B6 + scFv of 1A10 in Trispecific antibody 01
gatatccagatgacccagtctccttccagcctgtctgcctctgtgggcgacagagtgaccatcacatgcaaggccagccaggatgtgacccctgccgttgcttggtatcagcagaagcctggcaaggcccctaagctgctgatctactccacctcctccagatacacaggcgtgccctccagattctccggctctggctctggcaccgactttacctttacaatctccagcctgcagcctgaggatatcgccacctactactgccagcagcactacaccacacctctgacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctatagcctgtcctccacactgaccctgtccaaggccgactacgagaagcacaaggtgtacgcctgcgaagtgacccatcagggcctgtctagtcccgtgaccaagtctttcaacagaggcgagtgtggctccggcagcggaagtggttctggaagcggatctggttccggaagtggcagcggaggcggaggatctggtggcggaggaagtggcggaggcggtagtggtggtggtggatcaggtggtggcggatccggcggaggtggtagcggttctggttcaggatctggatctggcagcggctccggttctggatccgaagtgcagctggttgaatctggcggtggactggttcagcctggcggatctctgagactgtcttgtgccgcctccggcttcaccttctccagctacgatatgtcctgggtccgacaggcccctggcaagtctttggaatgggtcgccaccatctctgacgctggcggctacatctactaccgggactctgtgaagggcagattcaccatcagccgggacaacgccaagaactccctgtacctgcagatgaacagcctgcgcgacgaggacaccgccgtgtatatctgtgctagagagctgccttggagatacgccctggattattggggccagggaaccacggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccctgcctcctagcagagaagagatgaccaagaaccaggtgtccctgtcctgtgccgtgaagggcttctacccttccgatatcgccgtggaatgggagtccaatggccagcctgagaacaactacaagaccacacctcctgtgctggactccgacggctcattcttcctggtgtccaagctgaccgtggacaagtctaggtggcagcagggcaacgtgttctcctgctctgtgatgcacgaggccctgcacaaccactacacccagaagtccctgagcctgtctcctggaaaaggcggcggaggatctggcggaggtggaagcggaggcggtggatctcagtctgttctgacccagcctccttccgcttctggcacacctggacagagagtgaccatctcttgctccggctcctcctccaacatcggcaacaactacgtgacctggtatcagcagctgcccggcacagctcccaaactgctgatctacgccgactctcacagaccttccggcgtgcccgatagattctccggctctaagtctggcacctctgccagcctggctatctccggcctgagatctgaggacgaggccgactactactgcgccacctgggattattccctgtccggctacgtgttcggctgcggcacaaaactgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctccggctttaccttctcctcctacgacatgtcttgggtccgacaggcccctggcaagtgtctggaatgggtttcctggatctcctactccggcggctccatctactacgccgattctgtgaagggcagattcaccatcagccgggacaactccaagaacaccctgtacctgcagatgaactccctgagagccgaggacaccgccgtgtactactgtgccagagatgcccagcggaacagcatgagagagttcgactattggggccagggcaccctggtcacagtctcttct

SEQ ID NO: 257 - DNA, 642 bp, Artificial Sequence
  Light chain of B6 in Trispecific antibody 01
gatatccagatgacccagtctccttccagcctgtctgcctctgtgggcgacagagtgaccatcacatgcaaggccagccaggatgtgacccctgccgttgcttggtatcagcagaagcctggcaaggcccctaagctgctgatctactccacctcctccagatacacaggcgtgccctccagattctccggctctggctctggcaccgactttacctttacaatctccagcctgcagcctgaggatatcgccacctactactgccagcagcactacaccacacctctgacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctatagcctgtcctccacactgaccctgtccaaggccgactacgagaagcacaaggtgtacgcctgcgaagtgacccatcagggcctgtctagtcccgtgaccaagtctttcaacagaggcgagtgt

SEQ ID NO: 258 - DNA, 192 bp, Artificial Sequence
  Linker in Trispecific antibody 01
ggctccggcagcggaagtggttctggaagcggatctggttccggaagtggcagcggaggcggaggatctggtggcggaggaagtggcggaggcggtagtggtggtggtggatcaggtggtggcggatccggcggaggtggtagcggttctggttcaggatctggatctggcagcggctccggttctggatcc

SEQ ID NO: 259 - DNA, 1347 bp, Artificial Sequence
  Heavy chain of B6 in Trispecific antibody 01
gaagtgcagctggttgaatctggcggtggactggttcagcctggcggatctctgagactgtcttgtgccgcctccggcttcaccttctccagctacgatatgtcctgggtccgacaggcccctggcaagtctttggaatgggtcgccaccatctctgacgctggcggctacatctactaccgggactctgtgaagggcagattcaccatcagccgggacaacgccaagaactccctgtacctgcagatgaacagcctgcgcgacgaggacaccgccgtgtatatctgtgctagagagctgccttggagatacgccctggattattggggccagggaaccacggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccctgcctcctagcagagaagagatgaccaagaaccaggtgtccctgtcctgtgccgtgaagggcttctacccttccgatatcgccgtggaatgggagtccaatggccagcctgagaacaactacaagaccacacctcctgtgctggactccgacggctcattcttcctggtgtccaagctgaccgtggacaagtctaggtggcagcagggcaacgtgttctcctgctctgtgatgcacgaggccctgcacaaccactacacccagaagtccctgagcctgtctcctggaaaa

SEQ ID NO: 260 - DNA, 45 bp, Artificial Sequence; identical to SEQ ID NO: 253
  Linker in Trispecific antibody 01

SEQ ID NO: 261 - DNA, 753 bp, Artificial Sequence
  scFv of 1A10 in Trispecific antibody 01
cagtctgttctgacccagcctccttccgcttctggcacacctggacagagagtgaccatctcttgctccggctcctcctccaacatcggcaacaactacgtgacctggtatcagcagctgcccggcacagctcccaaactgctgatctacgccgactctcacagaccttccggcgtgcccgatagattctccggctctaagtctggcacctctgccagcctggctatctccggcctgagatctgaggacgaggccgactactactgcgccacctgggattattccctgtccggctacgtgttcggctgcggcacaaaactgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctccggctttaccttctcctcctacgacatgtcttgggtccgacaggcccctggcaagtgtctggaatgggtttcctggatctcctactccggcggctccatctactacgccgattctgtgaagggcagattcaccatcagccgggacaactccaagaacaccctgtacctgcagatgaactccctgagagccgaggacaccgccgtgtactactgtgccagagatgcccagcggaacagcatgagagagttcgactattggggccagggcaccctggtcacagtctcttct

SEQ ID NO: 262 - protein, 446 aa, Artificial Sequence; identical to SEQ ID NO: 215
  Heavy chain of (HC+LC) of C4I in Trispecific antibody 02

SEQ ID NO: 263 - protein, 15 aa, Artificial Sequence
  Linker of (HC+LC) of C4I in Trispecific antibody 02
GGGGSGGGGSGGGGS

SEQ ID NO: 264 - protein, 251 aa, Artificial Sequence; identical to SEQ ID NO: 244
  scFv of 1A10 in Trispecific antibody 02

SEQ ID NO: 265 - protein, 216 aa, Artificial Sequence; identical to SEQ ID NO: 178
  Light chain of (HC+LC) of C4I in Trispecific antibody 02

SEQ ID NO: 266 - protein, 993 aa, Artificial Sequence
  scFab-Fc of B12 + scFv of 1A10 in Trispecific antibody 02
DIQMTQSPSTLSASVGDRVIITCRASRGISSWLAWYQQKPGKAPNLLISKASSLESGVPSRFSGSGSGTDFTLTISSLQPEDFATYYCQQSSSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGSGSGSGSGSGSGSGSGGGGSGGGGSGGGGSGGGGSGGGGSGGGGSGSGSGSGSGSGSGSGSQVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 267 - protein, 214 aa, Artificial Sequence
  Light chain of B12 in Trispecific antibody 02
DIQMTQSPSTLSASVGDRVIITCRASRGISSWLAWYQQKPGKAPNLLISKASSLESGVPSRFSGSGSGTDFTLTISSLQPEDFATYYCQQSSSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 64 aa, Artificial Sequence; identical to SEQ ID NO: 201
  Linker in Trispecific antibody 02

SEQ ID NO: 269 - protein, 449 aa, Artificial Sequence
  Heavy chain of B12 in Trispecific antibody 02
QVQLLESGGGLVQPGGSLRLSCAASGFTFSSYWMSWVRQAPGKGLEWVANIKQDGSEKYYVDSVKGRFTISRDNAKNSLYLQMNSLRAEDTAVYYCARVALWDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 270 - protein, 15 aa, Artificial Sequence
  Linker in Trispecific antibody 02
GGGGSGGGGSGGGGS

SEQ ID NO: 271 - protein, 251 aa, Artificial Sequence; identical to SEQ ID NO: 244
  scFv of 1A10 in Trispecific antibody 02

SEQ ID NO: 272 - DNA, 1338 bp, Artificial Sequence; identical to SEQ ID NO: 252
  Heavy chain of (HC+LC) of C4I in Trispecific antibody 02

SEQ ID NO: 273 - DNA, 45 bp, Artificial Sequence; identical to SEQ ID NO: 253
  Linker of (HC+LC) of C4I in Trispecific antibody 02

SEQ ID NO: 274 - DNA, 753 bp, Artificial Sequence; identical to SEQ ID NO: 254
  scFv of 1A10 in Trispecific antibody 02

SEQ ID NO: 275 - DNA, 648 bp, Artificial Sequence; identical to SEQ ID NO: 152
  Light chain of (HC+LC) of C4I in Trispecific antibody 02

SEQ ID NO: 276 - DNA, 2979 bp, Artificial Sequence
  scFab-Fc of B12 + scFv of 1A10 in Trispecific antibody 02
gatatccagatgacccagtctccttccacactgtccgcctctgtgggcgacagagtgatcatcacctgtagagccagccggggcatctcttcttggctggcttggtatcagcagaagcccggcaaggcccctaacctgctgatctctaaggcctcctctctggaatccggcgtgcccagcagattttccggctctggctctggcaccgactttaccctgacaatctccagcctgcagcctgaggacttcgccacctactactgccagcagtccagcagcatccctctgacatttggcggaggcaccaaggtggaaatcaagcggacagtcgccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaacagaggcgagtgcggctccggaagcggaagtggatcaggttctggatctggcagcggtagcggcagtggtggcggaggttctggcggtggcggatcaggcggcggaggaagcggaggcggaggcagtggcggaggtggaagtggcggaggcggatctggaagtggaagcggttctggctcaggatctggttcaggctctggaagccaggtccagctgttggaatctggcggaggacttgttcagcctggcggctctctgagactgtcttgtgccgcttctggcttcaccttctccagctactggatgtcctgggtccgacaggctcctggcaaaggactggaatgggtcgccaacatcaagcaggacggctccgagaagtactacgtggactccgtgaagggcagattcaccatctctcgggacaacgccaagaactccctgtacctgcagatgaacagcctgagagccgaggacaccgccgtgtactactgtgctagagtggccctgtgggacgacgcctttgatatctggggacagggcacaatggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccctgcctcctagcagagaagagatgaccaagaaccaggtgtccctgtcctgtgccgtgaagggcttctacccttccgatatcgccgtggaatgggagtccaatggccagcctgagaacaactacaagaccacacctcctgtgctggactccgacggctcattcttcctggtgtccaagctgaccgtggacaagtctaggtggcagcagggcaacgtgttctcctgctctgtgatgcacgaggccctgcacaaccactacacccagaagtccctgagcctgtctcctggaaaaggcggcggaggatctggcggaggtggaagcggaggcggtggatctcagtctgttctgacccagcctccttccgcttctggcacacctggacagagagtgaccatctcttgctccggctcctcctccaacatcggcaacaactacgtgacctggtatcagcagctgcccggcacagctcccaaactgctgatctacgccgactctcacagaccttccggcgtgcccgatagattctccggctctaagtctggcacctctgccagcctggctatctccggcctgagatctgaggacgaggccgactactactgcgccacctgggattattccctgtccggctacgtgttcggctgcggcacaaaactgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctccggctttaccttctcctcctacgacatgtcttgggtccgacaggcccctggcaagtgtctggaatgggtttcctggatctcctactccggcggctccatctactacgccgattctgtgaagggcagattcaccatcagccgggacaactccaagaacaccctgtacctgcagatgaactccctgagagccgaggacaccgccgtgtactactgtgccagagatgcccagcggaacagcatgagagagttcgactattggggccagggcaccctggtcacagtctcttct

SEQ ID NO: 277 - DNA, 642 bp, Artificial Sequence
  Light chain of B12 in Trispecific antibody 02
gatatccagatgacccagtctccttccacactgtccgcctctgtgggcgacagagtgatcatcacctgtagagccagccggggcatctcttcttggctggcttggtatcagcagaagcccggcaaggcccctaacctgctgatctctaaggcctcctctctggaatccggcgtgcccagcagattttccggctctggctctggcaccgactttaccctgacaatctccagcctgcagcctgaggacttcgccacctactactgccagcagtccagcagcatccctctgacatttggcggaggcaccaaggtggaaatcaagcggacagtcgccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaacagaggcgagtgc

SEQ ID NO: 278 - DNA, 192 bp, Artificial Sequence
  Linker in Trispecific antibody 02
ggctccggaagcggaagtggatcaggttctggatctggcagcggtagcggcagtggtggcggaggttctggcggtggcggatcaggcggcggaggaagcggaggcggaggcagtggcggaggtggaagtggcggaggcggatctggaagtggaagcggttctggctcaggatctggttcaggctctggaagc

SEQ ID NO: 279 - DNA, 1347 bp, Artificial Sequence
  Heavy chain of B12 in Trispecific antibody 02
caggtccagctgttggaatctggcggaggacttgttcagcctggcggctctctgagactgtcttgtgccgcttctggcttcaccttctccagctactggatgtcctgggtccgacaggctcctggcaaaggactggaatgggtcgccaacatcaagcaggacggctccgagaagtactacgtggactccgtgaagggcagattcaccatctctcgggacaacgccaagaactccctgtacctgcagatgaacagcctgagagccgaggacaccgccgtgtactactgtgctagagtggccctgtgggacgacgcctttgatatctggggacagggcacaatggttaccgtgtcctctgcttctaccaagggacccagcgtgttccctctggctcctagctccaagtctacctctggcggaacagctgctctgggctgcctggtcaaggactactttcctgagcctgtgacagtgtcctggaactctggcgctctgacatctggcgtgcacacctttccagcagtgctgcagtcctccggcctgtactctctgtcctctgtcgtgacagtgccctcctctagcctgggcacccagacctacatctgcaatgtgaaccacaagccttccaacaccaaggtcgacaagaaggtggaacccaagtcctgcgacaagacccacacatgccctccatgtcctgctccagaactgctcggaggcccctccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtgtacaccctgcctcctagcagagaagagatgaccaagaaccaggtgtccctgtcctgtgccgtgaagggcttctacccttccgatatcgccgtggaatgggagtccaatggccagcctgagaacaactacaagaccacacctcctgtgctggactccgacggctcattcttcctggtgtccaagctgaccgtggacaagtctaggtggcagcagggcaacgtgttctcctgctctgtgatgcacgaggccctgcacaaccactacacccagaagtccctgagcctgtctcctggaaaa

SEQ ID NO: 280 - DNA, 45 bp, Artificial Sequence; identical to SEQ ID NO: 253
  Linker in Trispecific antibody 02

SEQ ID NO: 281 - DNA, 753 bp, Artificial Sequence; identical to SEQ ID NO: 261
  scFv of 1A10 in Trispecific antibody 02

SEQ ID NO: 282 - protein, 446 aa, Artificial Sequence; identical to SEQ ID NO: 165
  Synthetic Heavy chain (full-length)

SEQ ID NO: 283 - protein, 446 aa, Artificial Sequence; identical to SEQ ID NO: 173
  Synthetic Heavy chain (full-length)

SEQ ID NO: 284 - protein, 446 aa, Artificial Sequence
  Synthetic Heavy chain (full-length)
EVQLLESGGGLVQPGGSLRLSCAASGFTFSSYSMSWVRQAPGKGLEWVSGIYSDGSNTYYADSVKGRFTISRDNSKNTLYLQMNSLRAEDTAVYYCAKMLHRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 451 aa, Artificial Sequence
  Synthetic Heavy chain (full-length)
EVQLLESGGGLVQPGGSLRLSCAASGFTFSDYAMSWVRQAPGKGLEWVSGISPGGSNTYYADSVKGRFTISRDNSKNTLYLQMNSLRAEDTAVYYCAKDAWIARLLLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 446 aa, Artificial Sequence
  Synthetic Heavy chain (full-length)
EVQLLESGGGLVQPGGSLRLSCAASGFTFSSYGMSWVRQAPGKGLEWVSGIYSGGSSKYYADSVKGRFTISRDNSKNTLYLQMNSLRAEDTAVYYCAKNRLRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 216 aa, Artificial Sequence; identical to SEQ ID NO: 170
  Synthetic Light chain (full-length)

SEQ ID NO: 288 - protein, 216 aa, Artificial Sequence; identical to SEQ ID NO: 178
  Synthetic Light chain (full-length)

SEQ ID NO: 289 - protein, 216 aa, Artificial Sequence
  Synthetic Light chain (full-length)
QSVLTQPPSASGTPGQRVTISCSGSSSNIGSNSVTWYQQLPGTAPKLLIYADSQRPSGVPDRFSGSKSGTSASLAISGLRSEDEADYYCGTWDSSLNAYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 290 - protein, 216 aa, Artificial Sequence
  Synthetic Light chain (full-length)
QSVLTQPPSASGTPGQRVTISCSGSSSNIGSNAVTWYQQLPGTAPKLLIYYNNKRPSGVPDRFSGSKSGTSASLAISGLRSEDEADYYCGTWDDSLSGYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 291 - protein, 216 aa, Artificial Sequence
  Synthetic Light chain (full-length)
QSVLTQPPSASGTPGQRVTISCTGSSSNIGSNSVTWYQQLPGTAPKLLIYSDSHRPSGVPDRFSGSKSGTSASLAISGLRSEDEADYYCGTWDASLNAYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 292 - protein, 446 aa, Artificial Sequence
  Synthetic Heavy chain
EVQLLESGGGLVQPGGSLRLSCAASGFTFSSYSMSWVRQAPGKGLEWVSGIYSDASNTYYADSVKGRFTISRDNSKNTLYLQMNSLRAEDTAVYYCAKMLHRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 216 aa, Artificial Sequence
  Synthetic Light chain
QSVLTQPPSASGTPGQRVTISCSGSSSNIGSNSVTWYQQLPGTAPKLLIYADVQRPSGVPDRFSGSKSGTSASLAISGLRSEDEADYYCGTWDSSLNAYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 294 - protein, 5 aa, Artificial Sequence
  VH CDR1 of anti-PD-L1 antibody
SYWMS

SEQ ID NO: 295 - protein, 17 aa, Artificial Sequence
  VH CDR2 of anti-PD-L1 antibody
NIKQDGSEKYYVDSVKG

SEQ ID NO: 296 - protein, 10 aa, Artificial Sequence
  VH CDR3 of anti-PD-L1 antibody
VALWDDAFDI

SEQ ID NO: 297 - protein, 11 aa, Artificial Sequence
  VL CDR1 of anti-PD-L1 antibody
RASRGISSWLA

SEQ ID NO: 298 - protein, 7 aa, Artificial Sequence
  VL CDR2 of anti-PD-L1 antibody
KASSLES

SEQ ID NO: 299 - protein, 9 aa, Artificial Sequence
  VL CDR3 of anti-PD-L1 antibody
QQSSSIPLT

SEQ ID NO: 300 - protein, 712 aa, Artificial Sequence
  HC of C4I + scFv of 1A10 in Trispecific antibody 01
EVQLLESGGGLVQPGGSLRLSCAASGFTFSGYYMSWVRQAPGKGLEWVSLISPSSGSIYYADSVKGRFTISRDNSKNTLYLQMNSLRAEDTAVYYCAKGLTKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQSVLTQPPSASGTPGQRVTISCSGSSSNIGNNYVTWYQQLPGTAPKLLIYADSHRPSGVPDRFSGSKSGTSASLAISGLRSEDEADYYCATWDYSLSGYVFGCGTKLTVLGGGGSGGGGSGGGGSGGGGSEVQLLESGGGLVQPGGSLRLSCAASGFTFSSYDMSWVRQAPGKCLEWVSWISYSGGSIYYADSVKGRFTISRDNSKNTLYLQMNSLRAEDTAVYYCARDAQRNSMREFDYWGQGTLVTVSS

SEQ ID NO: 301 - DNA, 2136 bp, Artificial Sequence
  HC of C4I + scFv of 1A10 in Trispecific antibody 01
gaagttcagctgttggaatctggcggcggattggttcagcctggcggatctctgagactgtcttgtgccgcctctggcttcaccttctccggttactatatgtcctgggtccgacaggctcctggcaaaggactggaatgggtgtcccttattagtccaagctccggctctatctactacgccgactctgtgaagggcagattcaccatcagccgggacaactccaagaacaccctgtacctgcagatgaactccctgagagccgaggacaccgccgtgtactactgcgccaagggcttgacaaaatttgactattggggccagggcacactggttaccgtgtcctctgcttctaccaagggaccctctgtgttccctctggctccttccagcaagtctacctctggtggaaccgctgctctgggctgcctggtcaaggattactttcctgagcctgtgaccgtgtcttggaactccggtgctctgacatctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtccaccatgtcctgctccagaactgctcggcggtccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggctaagggccagcctcgggaacctcaagtgtacaccctgcctcctagcagagaagagatgaccaagaaccaggtgtccctgtggtgcctggtcaagggcttctacccttccgatatcgccgtggaatgggagtccaatggccagcctgagaacaactacaagaccacacctcctgtgctggactccgacggctcattcttcctgtactccaagctgaccgtggacaagtccagatggcagcagggcaacgtgttctcctgctccgtgatgcacgaggccctgcacaatcactacacccagaagtccctgtctctgtcccctggaaaaggcggcggaggatctggcggaggtggaagcggaggcggtggatctcagtctgttctgacccagcctccttccgcttctggcacacctggacagagagtgaccatctcttgctccggctcctcctccaacatcggcaacaactacgtgacctggtatcagcagctgcccggcacagctcccaaactgctgatctacgccgactctcacagaccttccggcgtgcccgatagattctccggctctaagtctggcacctctgccagcctggctatctccggcctgagatctgaggacgaggccgactactactgcgccacctgggattattccctgtccggctacgtgttcggctgcggcacaaaactgacagtgcttggaggtggtggtagtggtggtggcggttcaggtggcggaggaagcggcggaggcggatctgaagttcagctgttggaatctggcggcggactggttcaacctggcggatctctgagactgtcttgtgccgcctccggctttaccttctcctcctacgacatgtcttgggtccgacaggcccctggcaagtgtctggaatgggtttcctggatctcctactccggcggctccatctactacgccgattccgtgaagggcagattcaccatcagccgggacaactccaagaacaccctgtacctgcagatgaactccctgagagccgaggacaccgccgtgtactactgtgccagagatgcccagcggaacagcatgagagagttcgactattggggccagggcaccctggtcacagtctcttct

SEQ ID NO: 302 - protein, 712 aa, Artificial Sequence; identical to SEQ ID NO: 300
  HC of C4I + scFv of 1A10 in Trispecific antibody 02

SEQ ID NO: 303 - DNA, 2136 bp, Artificial Sequence; identical to SEQ ID NO: 301
  HC of C4I + scFv of 1A10 in Trispecific antibody 02